(19) United States
(12) Patent Application Publication
(10) Pub. No.: US 2024/0349601 A1
(43) Pub. Date: Oct. 17, 2024

(54) ORGANIC PHOTODETECTOR INCLUDING CONDENSED CYCLIC COMPOUND, ELECTRONIC APPARATUS INCLUDING THE ORGANIC PHOTODETECTOR, AND THE CONDENSED CYCLIC COMPOUND

(71) Applicants: SAMSUNG DISPLAY CO., LTD., Yongin-si (KR); Korea University Research And Business Foundation, Seoul (KR)

(72) Inventors: Daeho Lee, Yongin-si (KR); Han Young Woo, Seoul (KR); Hwasook Ryu, Yongin-si (KR); Seokgyu Yoon, Yongin-si (KR); Hyejin Jung, Yongin-si (KR); Chaeyeon Park, Seoul (KR); Venkata Suman Krishna Jonnadula, Seoul (KR); Min Hun Jee, Seoul (KR)

(21) Appl. No.: 18/614,333

(22) Filed: Mar. 22, 2024

(30) Foreign Application Priority Data
Mar. 24, 2023 (KR) 10-2023-0039069
Dec. 11, 2023 (KR) 10-2023-0178964

(51) Int. Cl. H10K 85/60; C07D 495/04; C07D 495/14; H10K 30/40

(52) U.S. Cl. CPC H10K 85/657; C07D 495/04; C07D 495/14; H10K 85/6576; H10K 30/40

(57) ABSTRACT

A condensed cyclic compound represented by Formula 1, an organic photodetector including the condensed cyclic compound. and an electronic apparatus including the organic photodetector are provided:

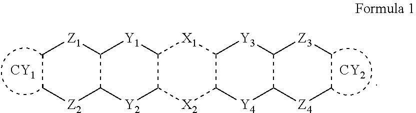

### CROSS-REFERENCE TO RELATED APPLICATION

The present application claims priority to and the benefit of Korean Patent Application Nos. 10-2023-0039069, filed on Mar. 24, 2023, and 10-2023-0178964, filed on Dec. 11, 2023, in the Korean Intellectual Property Office, the entire content of each of the two applications is incorporated herein by reference.

### BACKGROUND

### 1. Field

One or more embodiments of the present disclosure relate to an organic photodetector including a condensed cyclic compound, an electronic apparatus including the organic photodetector, and the condensed cyclic compound.

### 2. Description of the Related Art

Photoelectric devices are devices that convert light into an electrical signal and may include a photodiode and a phototransistor. Photoelectric devices may be applied to an image sensor, a solar cell, an organic light-emitting device, and/or the like.

In the case of silicon, which is commonly and mainly utilized in photodiodes, as the size of pixels decreases, an absorption region may decrease, thus deteriorating sensitivity. Accordingly, organic materials that may replace silicon are being proposed and/or studied.

As organic materials have a large extinction coefficient and may selectively absorb light in a specific wavelength region according to the molecular structure thereof, organic photodetectors including organic materials may replace comparable photodiodes and color filters simultaneously, which may facilitate improvements in sensitivity and high integration.

An organic photodetector (OPD) including such an organic material may be applied to, for example, a display apparatus or an image sensor.

### SUMMARY

One or more aspects of embodiments of the present disclosure are directed toward an organic photodetector including a condensed cyclic compound, an electronic apparatus including the organic photodetector, and the condensed cyclic compound.

Additional aspects will be set forth in part in the description which follows and, in part, will be apparent from the description, or may be learned by practice of the presented embodiments of the disclosure.

According to one or more embodiments of the present disclosure, provided is a condensed cyclic compound represented by Formula 1:

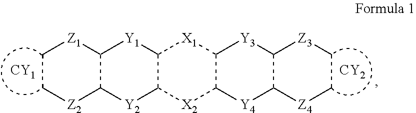

wherein, in Formula 1,

X₁ may be a single bond, C(R₁), C(R₁)(R₂), Si(R₁) (R₂), Ge(R₁)(R₂), N(R₁), B(R₁), O, S, Se, or Te,

X₂ may be a single bond, C(R₃), C(R₃) (R₄), Si(R₃) (R₄), Ge(R₃) (R₄), N(R₃), B(R₃), O, S, Se, or Te,

Y₁ to Y₄ may each independently be a single bond, O, S, Se, or Te,

Z₁ may be a single bond or

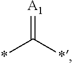

Z₂ may be a single bond or

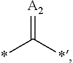

Z₃ may be a single bond or

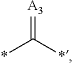

Z₄ may be a single bond or

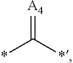

A₁ to A₄ may each independently be O, S, C(R₅)(R₆), or N(R₅),

at least one selected from among X₁ and X₂ may not be a single bond,

any one selected from among Y₁ and Y₂ may be a single bond, and the other may not be a single bond,

any one selected from among Y₃ and Y₄ may be a single bond, and the other may not be a single bond,

at least one selected from among Z₁ and Z₂ may not be a single bond,

at least one selected from among Z₃ and Z₄ may not be a single bond,

CY₁ and CY₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₁ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)2(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

* and *′ may each independently indicate a binding site to a neighboring carbon atom.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a structure of an organic photodetector according to one or more embodiments of the present disclosure;

FIG. 2 and FIG. 3 are each a schematic view of a structure of an electronic apparatus according to one or more embodiments of the present disclosure; and

FIG. 4A and FIG. 4B are each a view of an example of an electronic

apparatus according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

Because the present disclosure may have diverse modified embodiments, embodiments are illustrated in the drawings and are described in the detailed description for purposes of explaining the present disclosure. An effect and a characteristic of the disclosure, and a method of accomplishing these will be apparent when referring to embodiments described with reference to the drawings. The present disclosure may, however, be embodied in many different forms and should not be construed as limited to the embodiments set forth herein.

An expression utilized in the singular encompasses the expression of the plural, such as, the singular forms “a,” “an,” “one,” and “the” utilized herein are intended to include the plural forms, unless it has a clearly different meaning in the context. Further, the utilization of “may” when describing embodiments of the present disclosure refers to “one or more embodiments of the present disclosure”.

It will be further understood that the terms “comprise(s)/comprising,” “include(s)/including,” and/or “have(has)/having” utilized herein specify the presence of stated features or elements, but do not preclude the presence or addition of one or more other features or elements.

It will be understood that when a layer, region, or component is referred to as being “on” or “onto” another layer, region, or component, it may be directly or indirectly formed on the other layer, region, or component. That is, for example, intervening layers, regions, or components may be present. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In descriptions with reference to the drawings, identical or corresponding elements are assigned identical or like reference numerals, and overlapping descriptions thereof will not be provided for conciseness.

Sizes of elements in the drawings may be exaggerated for convenience of explanation. For example, because sizes and thicknesses of components in the drawings are arbitrarily illustrated for convenience of explanation, the following embodiments are not limited thereto.

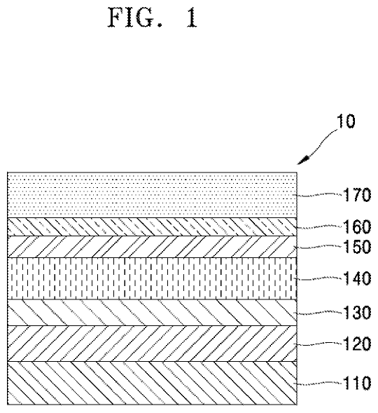

### Description of FIG. 1

FIG. 1 is a schematic cross-sectional view illustrating an organic photodetector 10 according to one or more embodiments of the present disclosure.

Referring to FIG. 1, the organic photodetector 10 according to one or more embodiments may include: a first electrode 110; a second electrode 170 facing the first electrode 110; an activation layer 140 between the first electrode 110 and the second electrode 170; an electron injection layer 160 between the activation layer 140 and the second electrode 170; an electron transport layer 150; a buffer layer; and a hole injection layer 120, a hole transport layer 130, and an auxiliary layer, which are between the first electrode 110 and the activation layer 140.

In FIG. 1, for example, in some embodiments, the electron injection layer 160, the electron transport layer 150, the buffer layer, the hole injection layer 120, the hole transport layer 130, and the auxiliary layer may each independently be present or may not each independently be present.

Recently, organic light-emitting devices with an organic photodetector have been developed as a sensor.

By utilizing organic materials included in the organic photodetector to absorb light of organic light-emitting devices having high emission efficiency, devices showing high absorption efficiency in the organic photodetector to which a common layer of an organic light-emitting device (e.g., a hole transport layer or an electron transport layer) is applied have been developed.

Materials such as monomers for deposition require a molecular weight of 1,500 g/mol or less. To absorb light of near-infrared region, a band gap of light absorbing molecule needs to be reduced by increasing effective conjugation. However, it is difficult to design a molecule absorbing light of near-infrared region by adjusting the length of the effective conjugation within a limited molecular weight. In one or more embodiments of the present disclosure, the organic

photodetector may include a condensed cyclic compound represented by Formula 1:

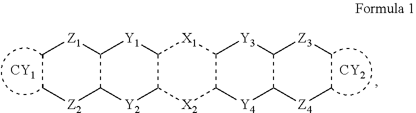

wherein, in Formula 1,

X₁ may be a single bond, C(R₁), C(R₁)(R₂), Si(R₁)(R₂), Ge(R₁)(R₂), N(R₁), B(R₁), O, S, Se, or Te,

X₂ may be a single bond, C(R₃), C(R₃)(R₄), Si(R₃) (R₄), Ge(R₃)(R₄), N(R₃), B(R₃), O, S, Se, or Te,

Y₁ to Y₄ may each independently be a single bond, O, S, Se, or Te,

Z₁ may be a single bond or

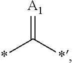

Z₂ may be a single bond or

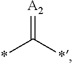

Z₃ may be a single bond or

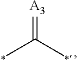

Z₄ may be a single bond or

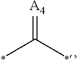

A₁ to A₄ may each independently be O, S, C(R₅)(R₆), or N(R₅),

at least one selected from among X₁ and X₂ may not be a single bond,

any one selected from among Y₁ and Y₂ may be a single bond, and the other may not be a single bond,

any one selected from among Ys and Y₄ may be a single bond, and the other may not be a single bond,

at least one selected from among Z₁ and Z₂ may not be a single bond, at least one selected from among Z₃ and Z₄ may not be a single bond,

CY₁ and CY₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₁ to R₆ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃),-N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁ to Q₃, Q₁₁ to Q₁₃, Q₂₁ to Q₂₃, and Q₃₁ to Q₃₃ may each independently be: hydrogen; deuterium; —F;—Cl; —Br;—I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

* and *′ may each independently indicate a binding site to a neighboring carbon atom.

In one or more embodiments, the condensed cyclic compound may have a symmetric structure.

In one or more embodiments, the condensed cyclic compound may have C2 symmetry. The term “C2 symmetry” may refer to that a compound is the same when rotated 180° about a central axis of the compound.

In one or more embodiments, the condensed cyclic compound may be axisymmetric with respect to a plane including X₁ and the plane may be perpendicular to a plane including X₁ and X₂.

In one or more embodiments, the condensed cyclic compound may have a molecular weight of 1,500 g/mol or less, 1,400 g/mol or less, 1,300 g/mol or less, 1,200 g/mol or less, 1,100 g/mol or less, or 1,000 g/mol or less. In some embodiments, the condensed cyclic compound may have a molecular weight of 250 g/mol to 1,500 g/mol, 250 g/mol to 1,400 g/mol, 250 g/mol to 1,300 g/mol, 250 g/mol to 1,200 g/mol, 250 g/mol to 1,100 g/mol, or 250 g/mol to 1,000 g/mol. As the condensed cyclic compound satisfies the foregoing molecular weights, deposition may be facilitated.

In one or more embodiments, the condensed cyclic compound may have a

highest occupied molecular orbital (HOMO) energy of −6.5 eV to −5.0 eV.

In one or more embodiments, the condensed cyclic compound may have a lowest unoccupied molecular orbital (LUMO) energy of −4.5 eV to −3.0 eV.

In one or more embodiments, the condensed cyclic compound may have an optical band gap of 2.5 eV or less or 2.2 eV or less. By satisfying the foregoing ranges, the condensed cyclic compound may absorb light of wide wavelength region even with a low molecular weight.

In one or more embodiments, the condensed cyclic compound may have a maximum emission wavelength of 600 nm to 800 nm, 610 nm to 790 nm, 620 nm to 780 nm, or 630 nm to 770 nm. The condensed cyclic compound may be suitable for absorbing infrared light or light of red wavelength region.

In one or more embodiments, the condensed cyclic compound may have a decomposition temperature of 200° C. or higher, 230° C. or higher, 260° C. or higher, or 290° C. or higher. As the condensed cyclic compound satisfies the foregoing decomposition temperature ranges, the condensed cyclic compound may maintain a stable structure even in a high-temperature deposition process.

In one or more embodiments, X₁ may be a single bond, C(R₁), C(R₁)(R₂), or N(R₁), X₂ may be a single bond, C(R₃), C(R₃)(R₄), or N(R₃), and at least one selected from among X₁ and X₂ may not be a single bond.

In one or more embodiments, X₁ may be a single bond, C(R₁)(R₂), or N(R₁), X₂ may be a single bond, C(R₃)(R₄), or N(R₃), any one selected from among X₁ and X₂ may be a single bond, and the other may not be a single bond.

In one or more embodiments, X₁ may be C(R₁), and X₂ may be C(R₃).

In one or more embodiments, Y₁ to Y₄ may each independently be a single bond or S, any one selected from among Y₁ and Y₂ may be a single bond, the other may not be a single bond, any one selected from among Y₃ and Y₄ may be a single bond, and the other may not be a single bond.

In one or more embodiments, Z₁ to Z₄ may each independently be a single bond,

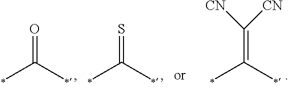

In one or more embodiments, any one selected from among Z₁ and Z₂ may be a single bond, the other may not be a single bond, any one selected from among Z₃ and Z₄ may be a single bond, and the other may not be a single bond.

In one or more embodiments, CY₁ and CY₂ may each independently be a cyclopentadiene group, an adamantane group, a norbornane group, a benzene group, a pentalene group, a naphthalene group, an azulene group, an indacene group, an acenaphthylene group, a phenylene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole group, a thiophene group, a thienothiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, an indazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group, each unsubstituted or substituted with at least one R₁₀ₐ.

In one or more embodiments, CY₁ and CY₂ may each independently be a cyclopentadiene group, a benzene group, a pentalene group, a naphthalene group, a phenalene group, a phenanthrene group, an anthracene group, a triphenylene group, a pyrene group, a chrysene group, a perylene group, a pentaphene group, a naphthacene group, an indene group, a fluorene group, a benzofluorene group, an indenophenanthrene group, an indenoanthracene group, a pyrrole group, a thiophene group, a thienothiophene group, a furan group, an indole group, a benzoindole group, a naphthoindole group, an isoindole group, a benzoisoindole group, a naphthoisoindole group, a benzothiophene group, a benzofuran group, a carbazole group, a dibenzothiophene group, a dibenzofuran group, a pyrazole group, an imidazole group, a triazole group, a thiazole group, an isothiazole group, a benzopyrazole group, a benzimidazole group, a benzoxazole group, a benzothiazole group, a benzoisothiazole group, an indazole group, a pyridine group, a pyrimidine group, a pyrazine group, a pyridazine group, a triazine group, a quinoline group, an isoquinoline group, a benzoquinoline group, or a benzoisoquinoline group, each unsubstituted or substituted with at least one R₁₀ₐ.

In one or more embodiments, R₁ to R₆ may each independently be:

hydrogen, deuterium, —F, −Cl, or a cyano group;

a C₁-C₆₀ alkyl group unsubstituted or substituted with at least one R₁₀ₐ or a C₁-C₆₀ alkoxy group unsubstituted or substituted with at least one R₁₀ₐ;

a C₃-C₆₀ carbocyclic group unsubstituted or substituted with at least one R₁₀ₐ or a C₁-C₆₀ heterocyclic group unsubstituted or substituted with at least one R₁₀ₐ; or

—C(Q₁) (Q₂)(Q₃), —Si(Q₁) (Q₂)(Q₃), —N(Q₁)(Q₂), —B(Q₁)(Q₂), —C(═O)(Q₁), —S(═O)₂(Q₁), or —P(═O)(Q₁)(Q₂).

In one or more embodiments, R₁ to R₆ may each independently be: hydrogen, deuterium, —F, —Cl, or a cyano group; or

a C₁-C₂₀ alkyl group or a C₁-C₂₀ alkoxy group, each unsubstituted or substituted with hydrogen, deuterium, —F, a cyano group, or any combination thereof.

In one or more embodiments, R₁ to R₆ may each independently be:

hydrogen, deuterium, —F, —Cl, or a cyano group;

a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2,2-dimethylpropyl group, a 1-ethylpropyl group, or a 1,2-dimethylpropyl group, each unsubstituted or substituted with deuterium, —F, —Cl, —Br, —I, a hydroxyl group, a cyano group, a nitro group, or any combination thereof; or a methoxy group, an ethoxy group, an n-propoxy group, an iso-propoxy

group, an n-butoxy group, an isobutoxy group, a sec-butoxy group, a tert-butoxy group, an n-pentoxy group, an iso-pentoxy group, a sec-pentoxy group, a tert-pentoxy group, a 2-methylbutoxy group, a 2,2-dimethylpropoxy group, a 1-ethylpropoxy group, or a 1,2-dimethylpropoxy group, each unsubstituted or substituted with deuterium, —F, —Cl, —Br, —I, a hydroxyl group, a cyano group, a nitro group, or any combination thereof.

In one or more embodiments, the condensed cyclic compound may be represented by any one of Formulae 1-1 to 1-7:

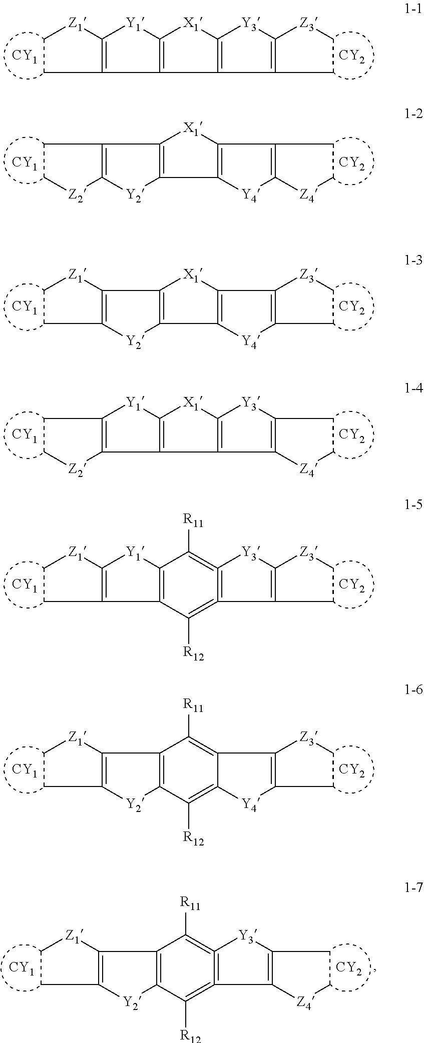

wherein, in Formulae 1-1 to 1-7,

CY₁ and CY₂ may be the same as described herein,

X₁′ may be C(R₁), C(R₁)(R₂), Si(R₁)(R₂), Ge(R₁)(R₂), N(R₁), B(R₁), O, S, Se, or Te,

Y₁′ to Y₄′ may each independently be O, S, Se, or Te,

Z₁′ may be

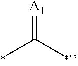

Z₂′ may be

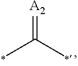

Z₃′ may be

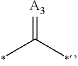

Z₄′ may be

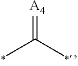

A₁ to A₄ may each independently be O, S, C(R₅)(R₆), or N(R₅), and

R₁₁ to R₁₂ may each be the same as described herein with respect to R₁.

In one or more embodiments, CY₁ and CY₂ may each independently be represented by any one selected from among Formulae 2-1 to 2-7:

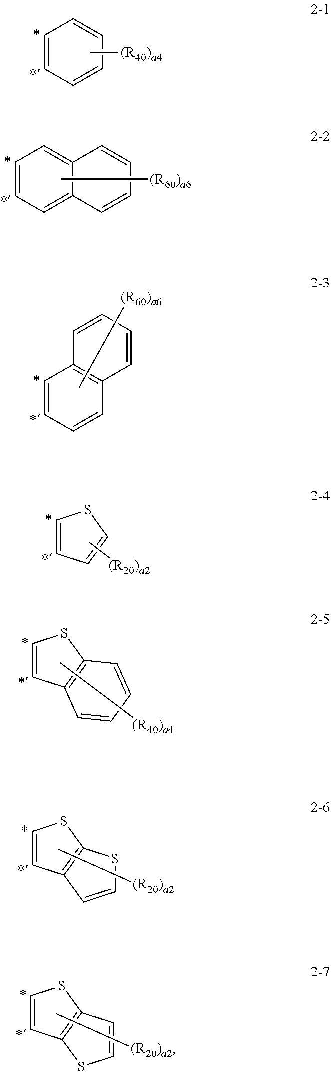

wherein, in Formulae 2-1 to 2-7,

R₂₀, R₄₀, and R₆₀ may each be the same as described herein with respect to R₁,

a2 may be 1 or 2,

a4 may be an integer from 1 to 4,

a6 may be an integer from 1 to 6, and

* and ** may each independently be carbon (e.g., a site) condensed with a neighboring ring.

In one or more embodiments, the condensed cyclic compound represented by Formula 1 may be any one selected from among Compounds 1 to 72:

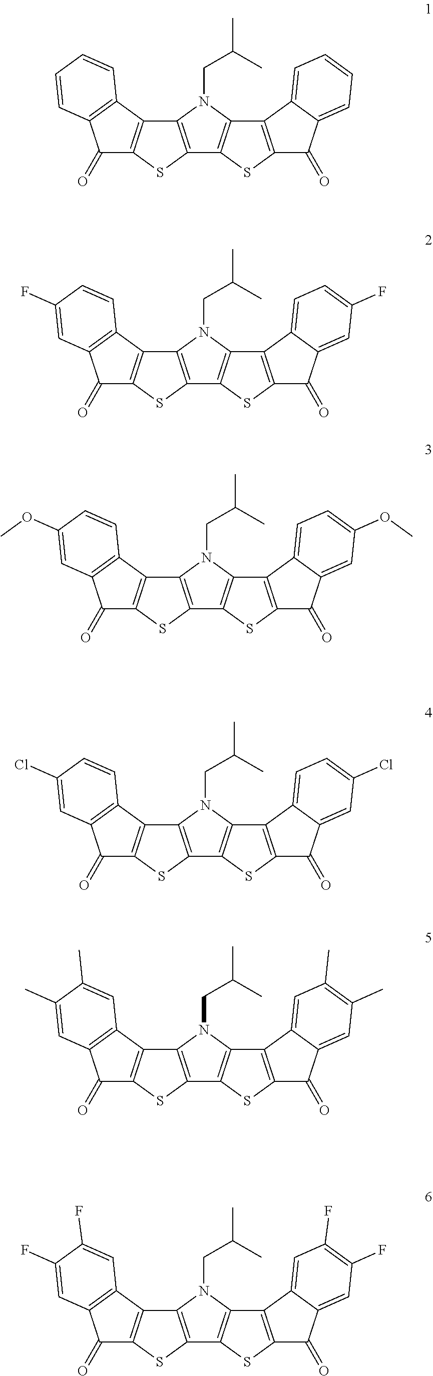

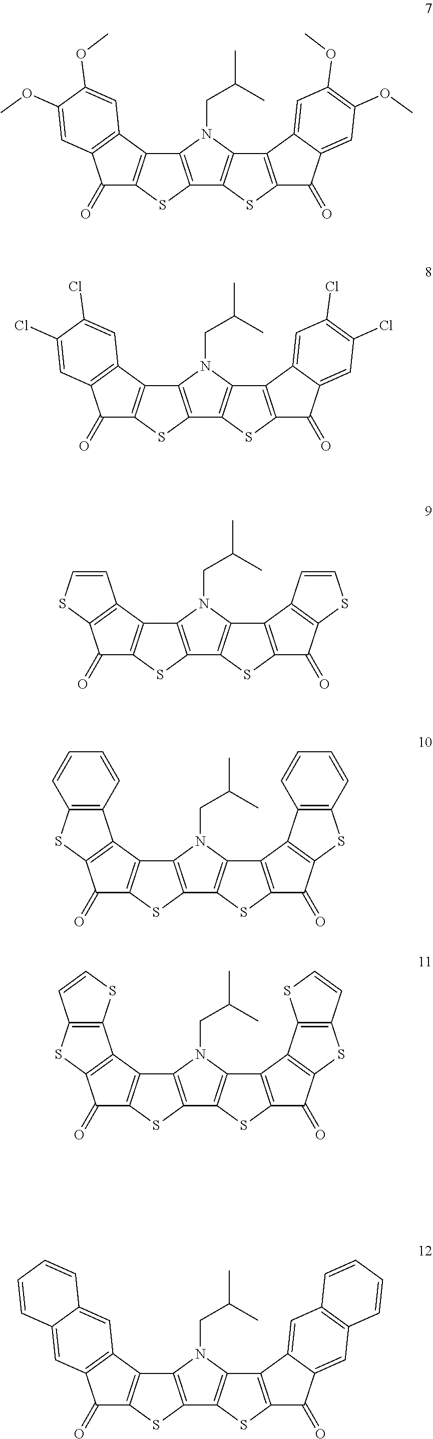

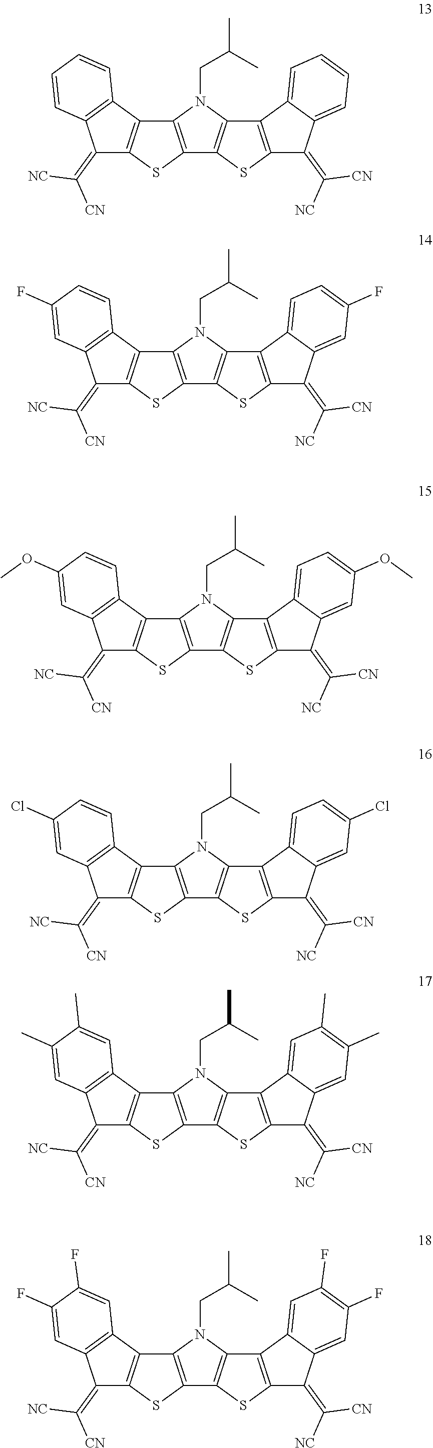

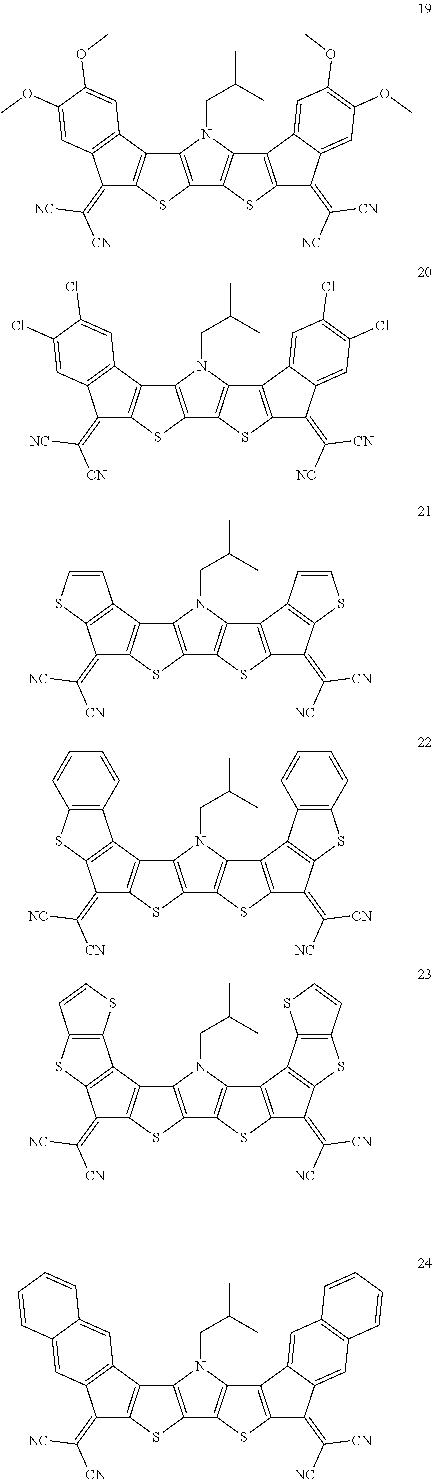

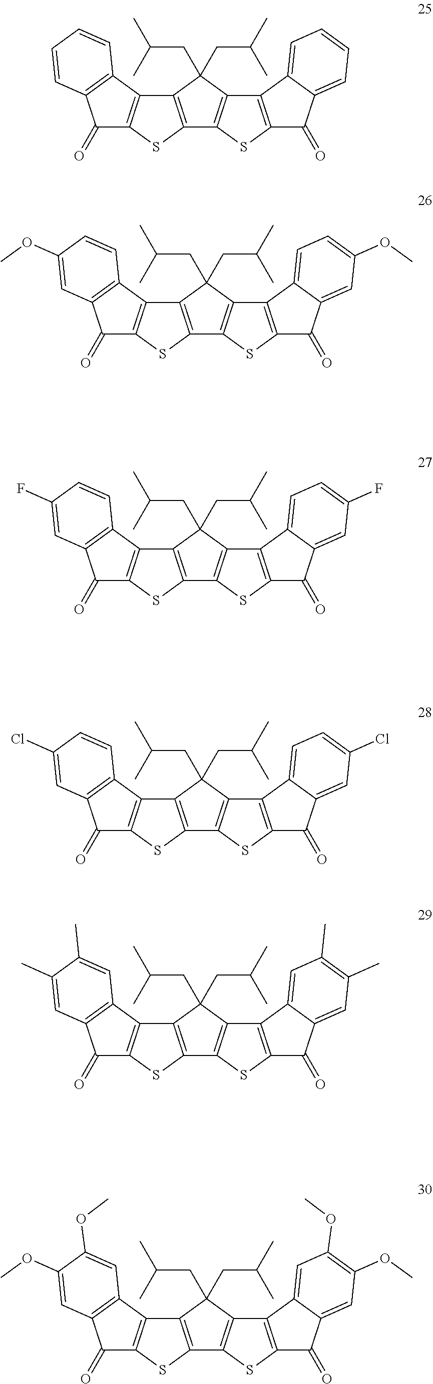

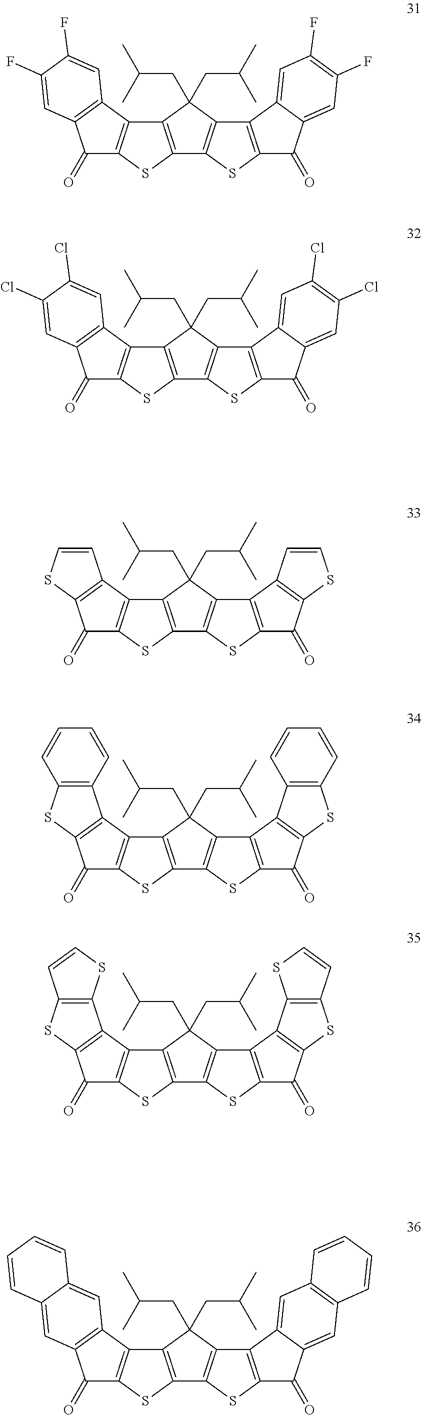 [SMILES: CC(C)CC1(CC(C)C)c2c(sc3c2-c2cc(F)c(F)cc2C3=O)-c2sc3c(c21)-c1cc(F)c(F)cc1C3=O ; CC(C)CC1(CC(C)C)c2c(sc3c2-c2cc(Cl)c(Cl)cc2C3=O)-c2sc3c(c21)-c1cc(Cl)c(Cl)cc1C3=O ; CC(C)CC1(CC(C)C)c2c(sc3c2-c2ccsc2C3=O)-c2sc3c(c21)-c1ccsc1C3=O ; CC(C)CC1(CC(C)C)c2c(sc3c2-c2c(sc4ccccc24)C3=O)-c2sc3c(c21)-c1c(sc2ccccc12)C3=O ; CC(C)CC1(CC(C)C)c2c(sc3c2-c2c(sc4ccsc24)C3=O)-c2sc3c(c21)-c1c(sc2ccsc12)C3=O ; CC(C)CC1(CC(C)C)c2c(sc3c2-c2cc4ccccc4cc2C3=O)-c2sc3c(c21)-c1cc2ccccc2cc1C3=O]

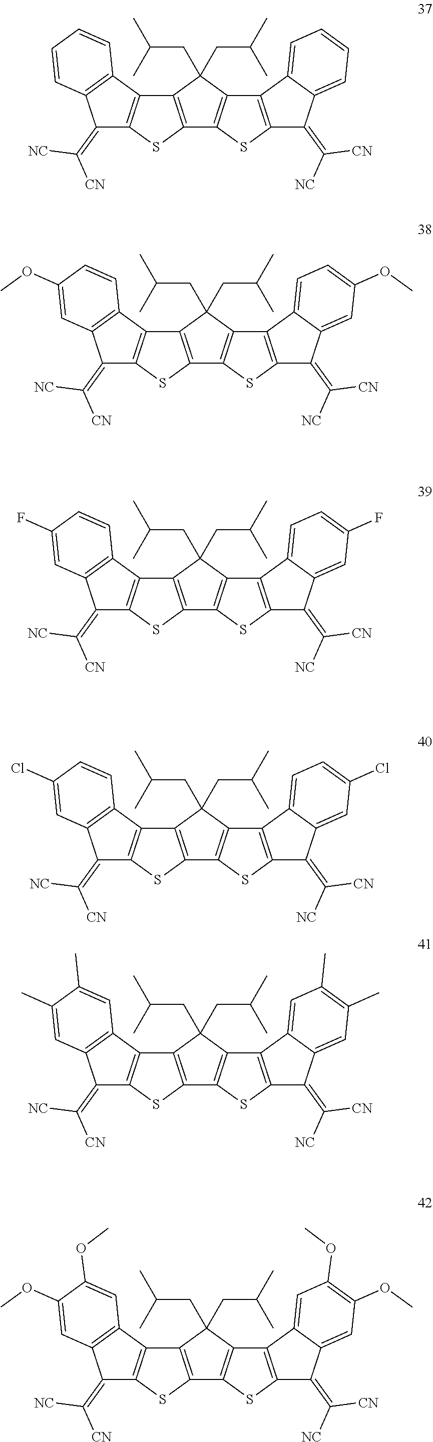

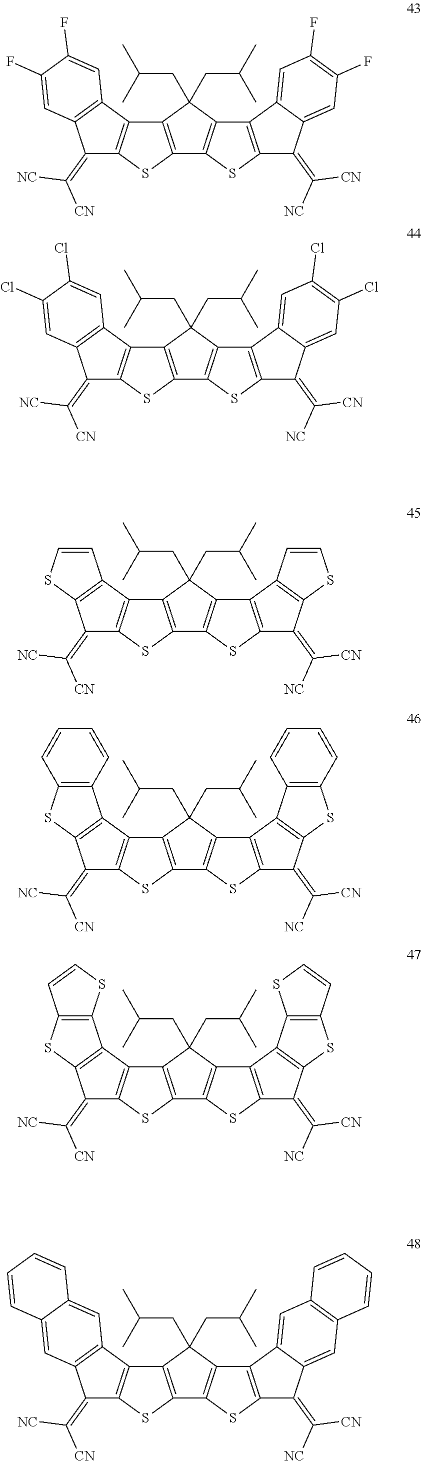

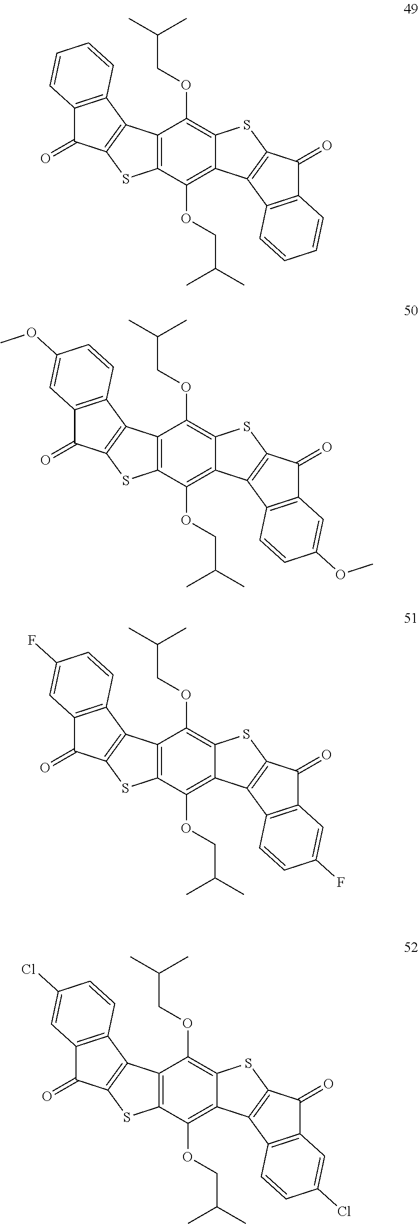

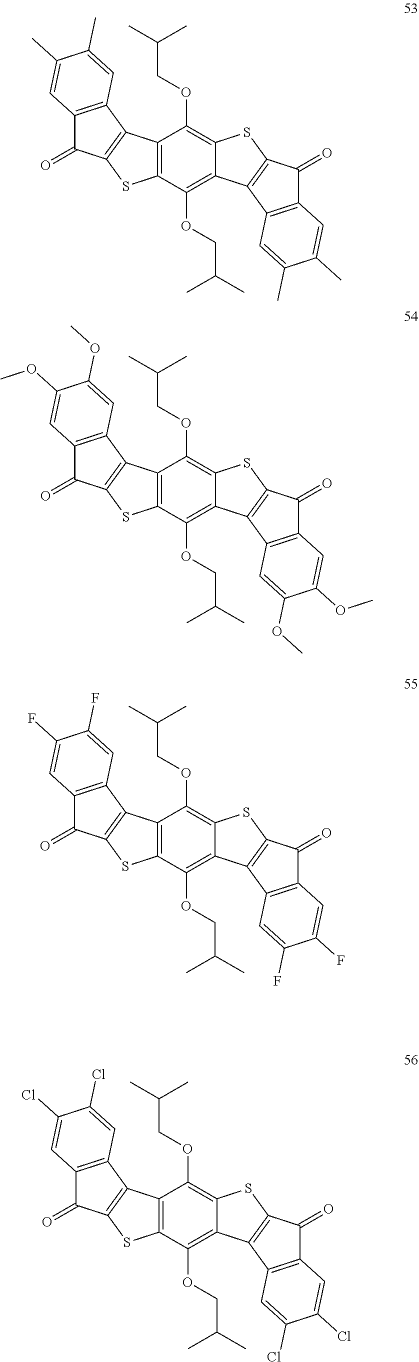

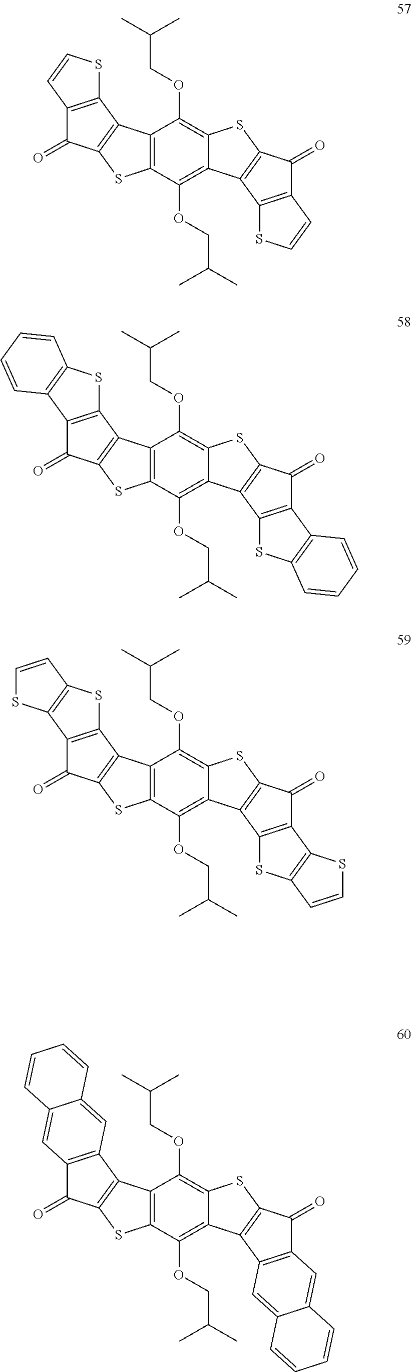

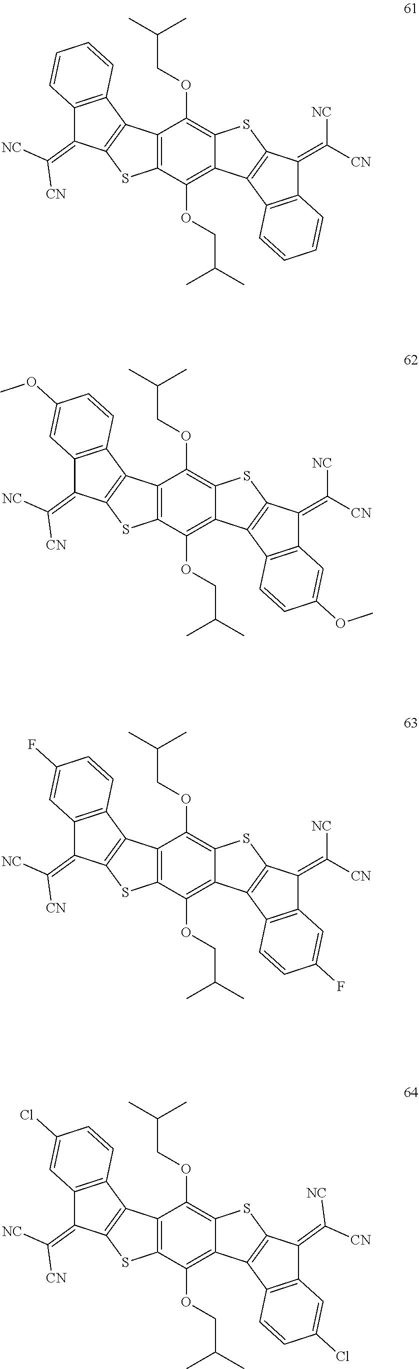

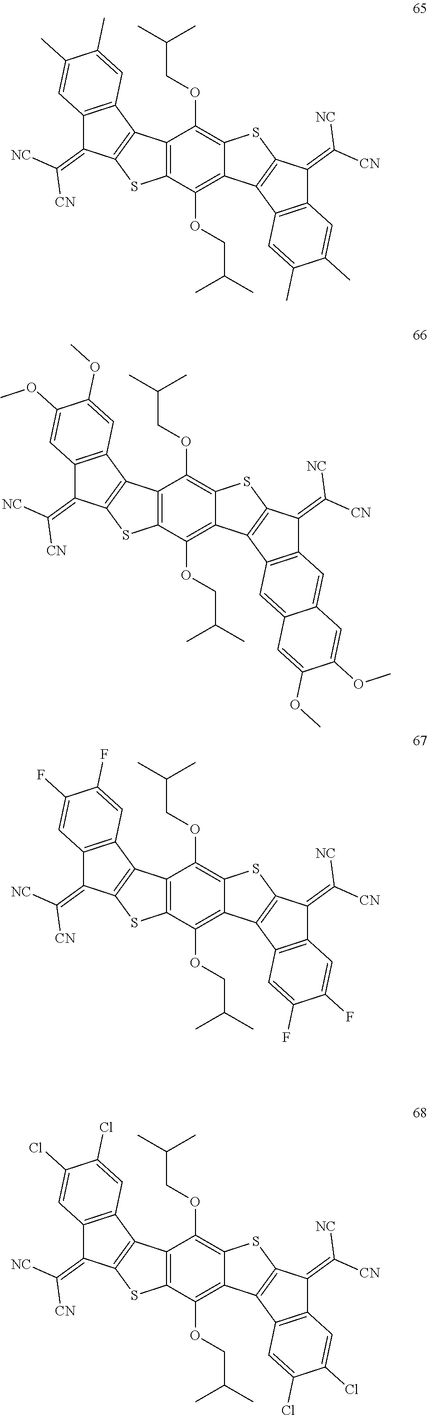

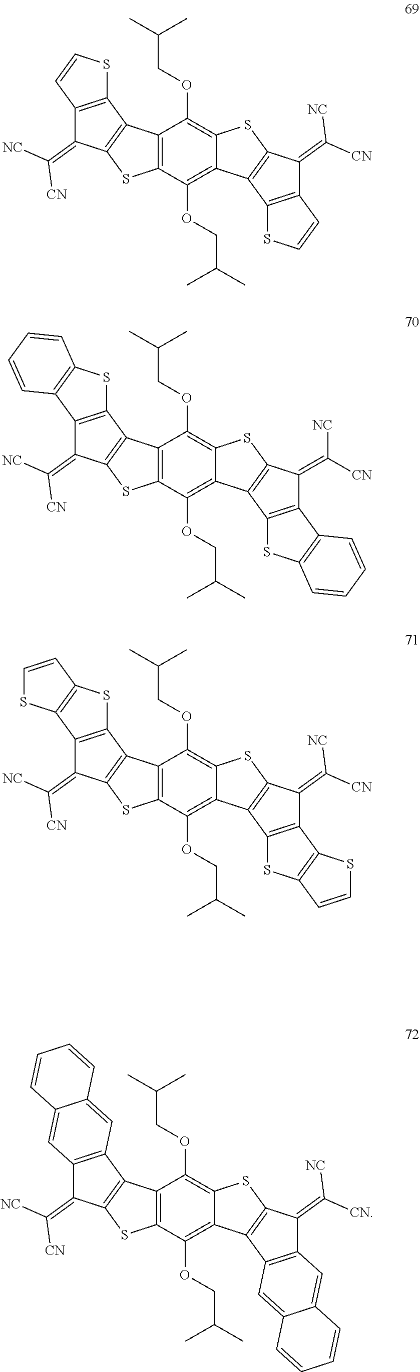

The condensed cyclic compound represented by Formula 1 may have a structure in which at least 7 rings are condensed, wherein Y₁ to Y₄ may each independently be a single bond, O, S, Se, or Te, any one selected from among Y₁ and Y₂ may be a single bond, and the other may not be a single bond, any one selected from among Y₃ and Y₄ may be a single bond, and the other may not be a single bond. That is, a ring condensed with a middle ring (ring including X₁ and X₂) may satisfy a 5-membered ring including a heteroatom.

In one or more embodiments, a ring condensed with the 5-membered ring including a heteroatom may include a suitable functional group such as Z₁ to Z₄.

Accordingly, the condensed cyclic compound represented by Formula 1 may have a structure with improved stability against external factors such as heat, light, external compounds, etc., compared to molecules of related arts. In one or more embodiments, based on its stable structure, the condensed cyclic compound may be employed in a photodetector having improved reliability. Moreover, according to the condensed cyclic compound, as the band gap may be easily increased or decreased by adjusting the effective conjugation, a person skilled in the art may easily manufacture a device or a sensor absorbing light in various wavelength regions (for example, in infrared and/or red region) by utilizing the condensed cyclic compound.

In one or more embodiments, the organic photodetector may include: a first electrode; a second electrode facing the first electrode; an activation layer between the first electrode and the second electrode; and the condensed cyclic compound.

In one or more embodiments, the condensed cyclic compound may be in the activation layer.

The activation layer may generate excitons by receiving light from the outside and separate the generated excitons into holes and electrons. The activation layer may include the condensed cyclic compound represented by Formula 1. In one or more embodiments, the activation layer may include an additional electron donor and/or electron accepter.

In one or more embodiments, the activation layer may include the condensed cyclic compound represented by Formula 1 as an electron acceptor or an electron donor.

In one or more embodiments, the condensed cyclic compound represented by Formula 1 may act as an electron accepter or an electron donor. In one or more embodiments, the activation layer may include: the condensed cyclic compound represented by Formula 1; and an electron donor or an electron accepter.

In one or more embodiments, the activation layer may include: a layer including the condensed cyclic compound represented by Formula 1; and a layer including an electron donor or an electron accepter. In these embodiments, the condensed cyclic compound represented by Formula 1 may respectively act as an electron accepter or an electron donor.

In one or more embodiments, the layer including the condensed cyclic compound represented by Formula 1 may be in contact with the layer including an electron donor or an electron accepter. For example, the layer including the condensed cyclic compound represented by Formula 1 may be in direct physical contact with the layer including an electron donor or an electron accepter.

In one or more embodiments, the layer including the condensed cyclic compound represented by Formula 1 and the layer including an electron donor or an electron accepter may form a PN junction. Excitons may be efficiently separated into holes and electrons by photo-induced charge separation occurring at an interface between these layers. Furthermore, as the activation layer is separated (e.g., divided) into the layer including the condensed cyclic compound represented by Formula 1 and the layer including an electron donor or an electron accepter, holes and electrons generated at the interface may be easily trapped or separated or may easily migrate.

In one or more embodiments, the activation layer may include a layer including a mixture of the condensed cyclic compound represented by Formula 1 and an electron donor or an electron accepter. In these embodiments, the condensed cyclic compound represented by Formula 1 may respectively act as an electron accepter or an electron donor. In these embodiments, the activation layer may be formed by co-depositing the compound represented by Formula 1 and an electron donor or an electron acceptor. When the activation layer is a mixed layer, excitons may be generated with a diffusion distance from a donor-acceptor interface, and thus, the organic photodetector may have improved external quantum efficiency. A ratio of the condensed cyclic compound represented by Formula 1 and the electron donor or the electron acceptor may be, for example, in a range of 10:90 to 90:10 (weight ratio).

In one or more embodiments, the activation layer may include a p-dopant. The p-dopant may be substantially homogeneously or non-homogeneously dispersed in the activation layer. When the activation layer is doped with the p-dopant, external quantum efficiency may be improved by the charge injection principle by an electric field.

In one or more embodiments, the electron donor may be an organic or inorganic material having a LUMO energy level deeper than −2 eV and a HOMO energy level deeper than −3 eV. In one or more embodiments, the electron donor may be an organic or inorganic material having a LUMO energy level of about −5 eV to about −3 eV and a HOMO energy level of about −7 eV to about −4 eV. In one or more embodiments, the electron donor may be boron subphthalocyanine chloride (SubPc), copper(II) phthalocyanine (CuPc), tetraphenyldibenzoperiflanthene (DBP), or any combination thereof.

In one or more embodiments, the electron acceptor may be an organic or inorganic material having a LUMO energy level deeper than about −3 eV and a HOMO energy level deeper than about −4 eV. In one or more embodiments, the electron acceptor may be an organic or inorganic material having a LUMO energy level of about −6 eV to about −4 eV and a HOMO energy level of about −8 eV to about −5 eV. For example, in some embodiments, the electron accepter may be C60 fullerene, 1,4,5,8,9,11-hexaazatriphenylenehexacarbonitrile (HATCN or HAT-CN), tetracyanoquinodimethane (TCNQ), diimide-based non-fullerene, etc.

As the diimide-based non-fullerene compound, for example, a compound represented by Formula 5 may be utilized:

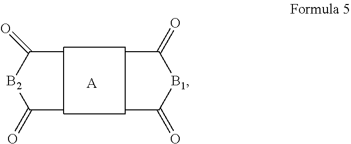

wherein, in Formula 5,

A may be a C₃-C₆₀ carbocyclic group unsubstituted or substituted with at least one R₁₀ₐ or a C₁-C₆₀ heterocyclic group unsubstituted or substituted with at least one R₁₀ₐ, and

B₁ and B₂ may each independently be oxygen or —NR₁₀—, wherein R₁₀ may be hydrogen, a C₁-C₆₀ alkyl group unsubstituted or substituted with at least one R₁₀ₐ, a C₃-C₁₀ cycloalkyl group unsubstituted or substituted with at least one R₁₀ₐ, a halogen, or a halogen-containing group.

Examples of Formula 5 are as provided herein; however, embodiments of the present disclosure are not limited thereto:

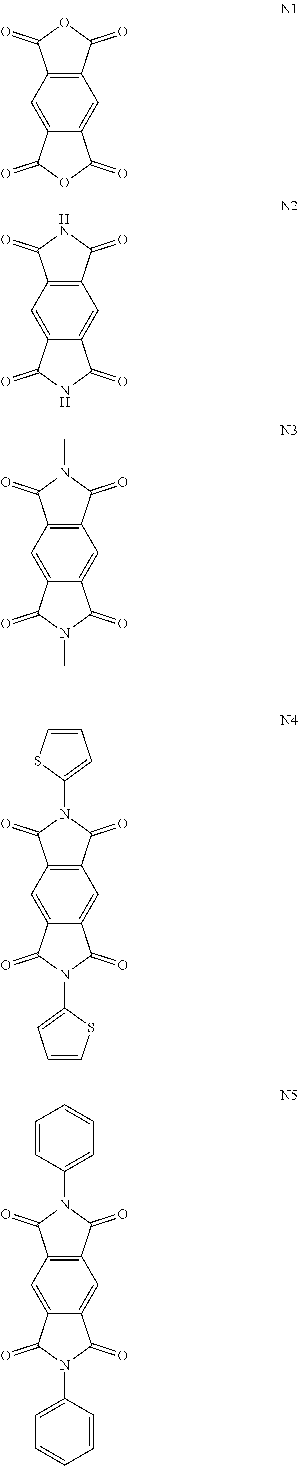 [SMILES: O=c1oc(=O)c2cc3c(=O)oc(=O)c3cc12 ; O=c1[nH]c(=O)c2cc3c(=O)[nH]c(=O)c3cc12 ; Cn1c(=O)c2cc3c(=O)n(C)c(=O)c3cc2c1=O ; O=c1c2cc3c(=O)n(-c4cccs4)c(=O)c3cc2c(=O)n1-c1cccs1 ; O=c1c2cc3c(=O)n(-c4ccccc4)c(=O)c3cc2c(=O)n1-c1ccccc1]

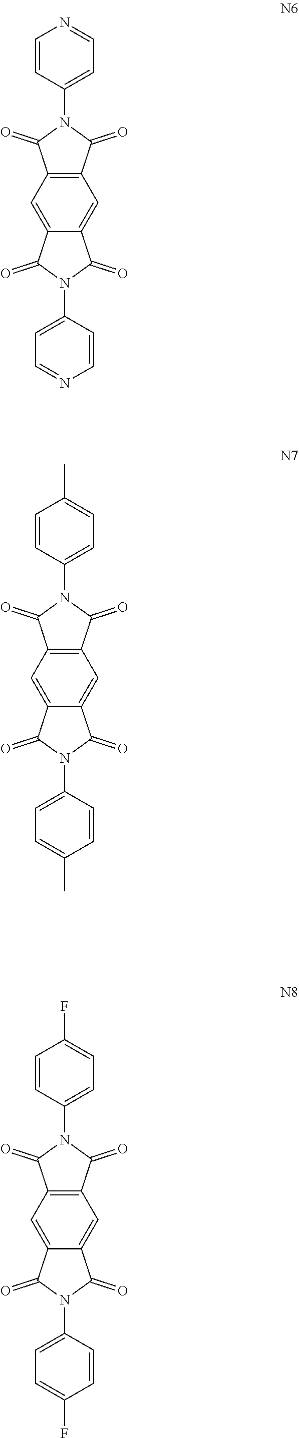 [SMILES: O=c1c2cc3c(=O)n(-c4ccncc4)c(=O)c3cc2c(=O)n1-c1ccncc1 ; Cc1ccc(-n2c(=O)c3cc4c(=O)n(-c5ccc(C)cc5)c(=O)c4cc3c2=O)cc1 ; O=c1c2cc3c(=O)n(-c4ccc(F)cc4)c(=O)c3cc2c(=O)n1-c1ccc(F)cc1]

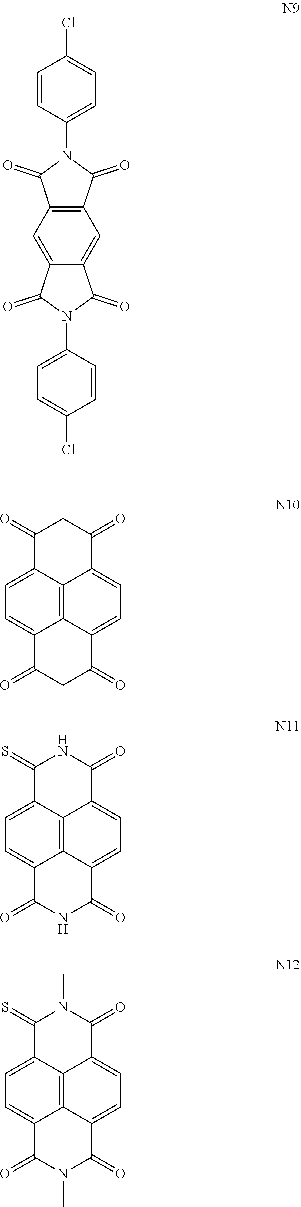 [SMILES: O=c1c2cc3c(=O)n(-c4ccc(Cl)cc4)c(=O)c3cc2c(=O)n1-c1ccc(Cl)cc1 ; O=C1CC(=O)c2ccc3c4c(ccc1c24)C(=O)CC3=O ; O=C1NC(=O)c2ccc3c4c(ccc1c24)C(=O)NC3=S ; CN1C(=O)c2ccc3c4c(ccc(c24)C1=O)C(=S)N(C)C3=O]

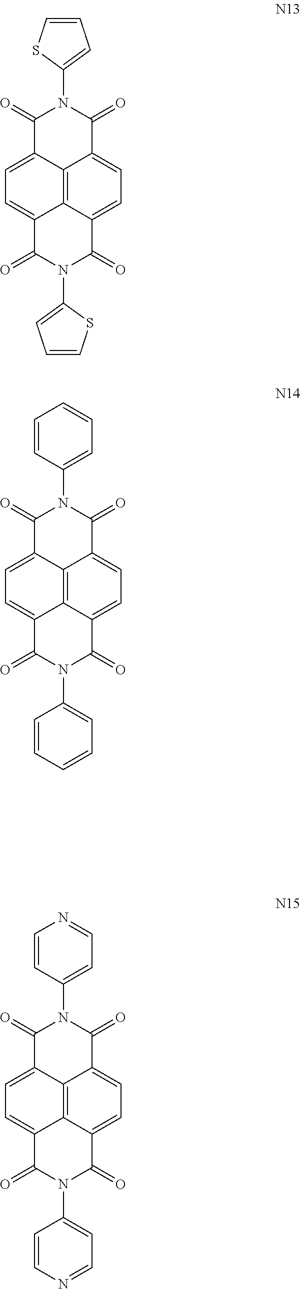 [SMILES: O=C1c2ccc3c4c(ccc(c24)C(=O)N1c1cccs1)C(=O)N(c1cccs1)C3=O ; O=C1c2ccc3c4c(ccc(c24)C(=O)N1c1ccccc1)C(=O)N(c1ccccc1)C3=O ; O=C1c2ccc3c4c(ccc(c24)C(=O)N1c1ccncc1)C(=O)N(c1ccncc1)C3=O]

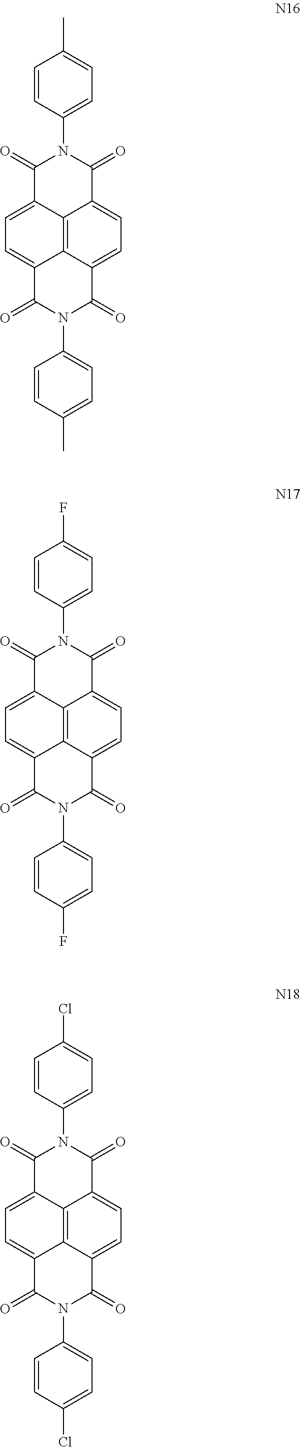 [SMILES: Cc1ccc(N2C(=O)c3ccc4c5c(ccc(c35)C2=O)C(=O)N(c2ccc(C)cc2)C4=O)cc1 ; O=C1c2ccc3c4c(ccc(c24)C(=O)N1c1ccc(F)cc1)C(=O)N(c1ccc(F)cc1)C3=O ; O=C1c2ccc3c4c(ccc(c24)C(=O)N1c1ccc(Cl)cc1)C(=O)N(c1ccc(Cl)cc1)C3=O]

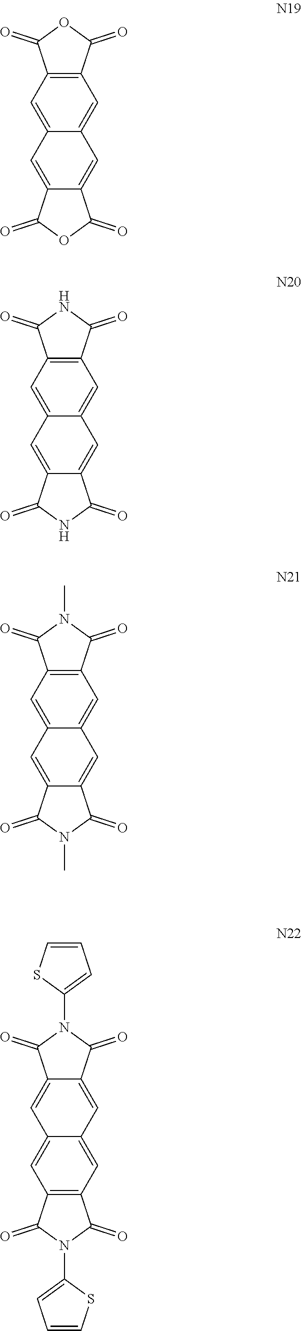 [SMILES: O=c1oc(=O)c2cc3cc4c(=O)oc(=O)c4cc3cc12 ; O=c1[nH]c(=O)c2cc3cc4c(=O)[nH]c(=O)c4cc3cc12 ; Cn1c(=O)c2cc3cc4c(=O)n(C)c(=O)c4cc3cc2c1=O ; O=c1c2cc3cc4c(=O)n(-c5cccs5)c(=O)c4cc3cc2c(=O)n1-c1cccs1]

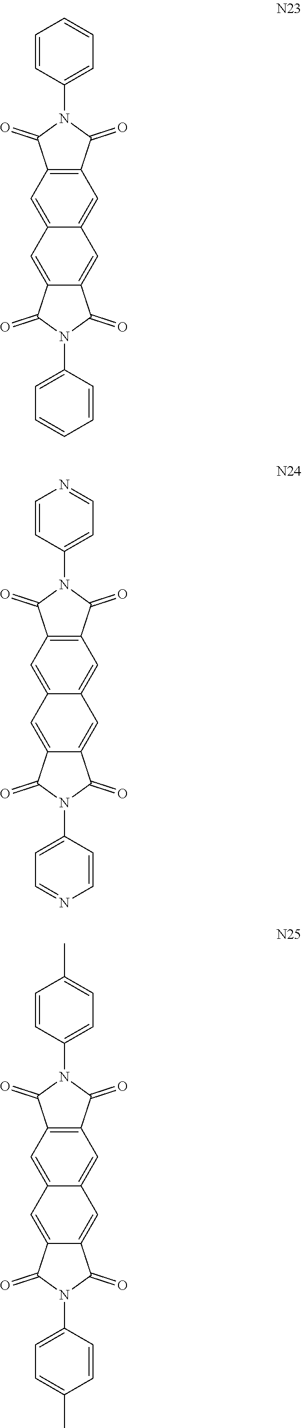

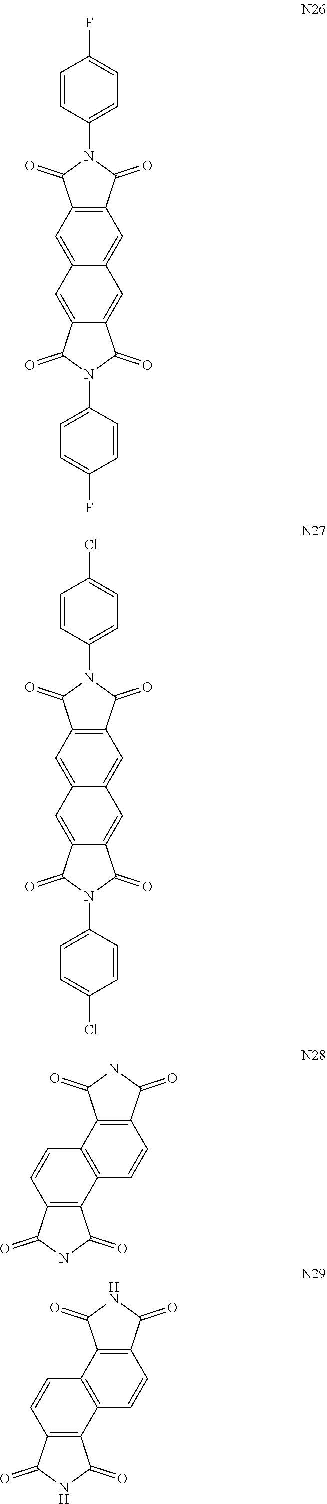

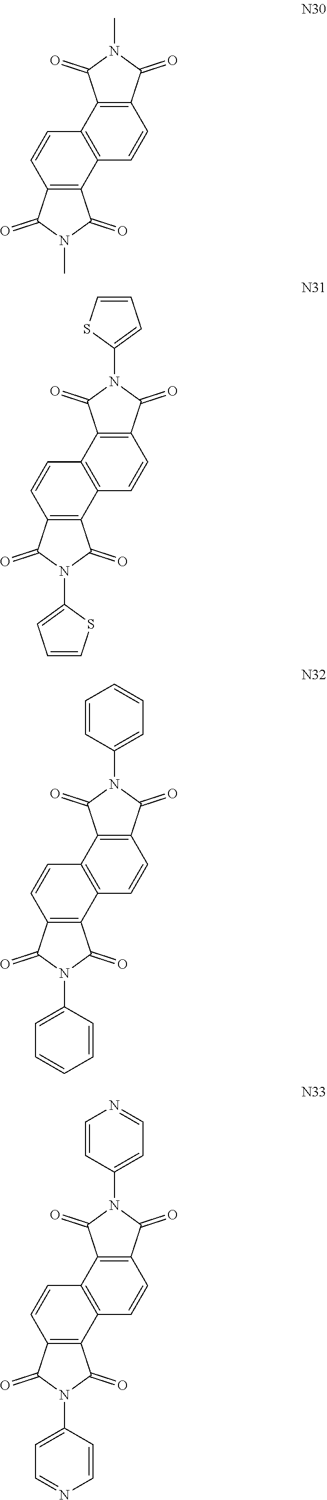

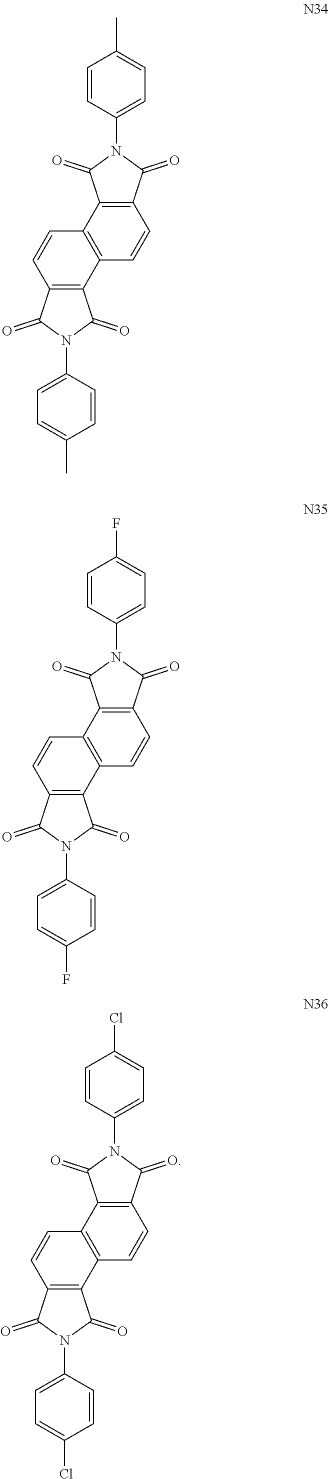 [SMILES: Cc1ccc(-n2c(=O)c3ccc4c(ccc5c(=O)n(-c6ccc(C)cc6)c(=O)c54)c3c2=O)cc1 ; O=c1c2ccc3c(ccc4c(=O)n(-c5ccc(F)cc5)c(=O)c43)c2c(=O)n1-c1ccc(F)cc1 ; O=c1c2ccc3c(ccc4c(=O)n(-c5ccc(Cl)cc5)c(=O)c43)c2c(=O)n1-c1ccc(Cl)cc1]

One of the first electrode 110 and the second electrode 170 may be an anode, and the other one may be a cathode. For example, in one or more embodiments, the first electrode 110 may be an anode, and the second electrode 170 may be a cathode.

A hole transport region of the organic photodetector 10 may include a structure in which the hole injection layer 120, the hole transport layer 130, an auxiliary layer, an electron blocking layer, or any combination thereof are arranged on the first electrode 110. In one or more embodiments, the auxiliary layer may be located between the hole transport layer 130 and the activation layer 140.

An electron transport region of the organic photodetector 10 may include a structure in which a buffer layer, a hole blocking layer, the electron transport layer 150, the electron injection layer 160, or any combination thereof are arranged on the activation layer 140. In one or more embodiments, the buffer layer may be located between the electron transport layer 150 and the activation layer 140.

In one or more embodiments, the p-dopant may include a quinone derivative, a cyano group-containing compound, a compound including element EL1 and element EL2, or any combination thereof.

Non-limiting examples of the quinone derivative may be TCNQ, 2,3,5,6-tetrafluoro-7,7,8,8-tetracyanoquinodimethane (F4-TCNQ), etc.

Non-limiting examples of the cyano group-containing compound may be HAT-CN, and a compound represented by Formula 221:

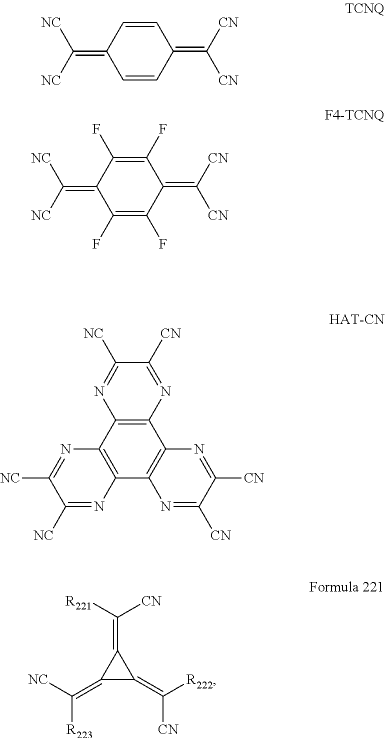

wherein, in Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from among R₂₂₁ to R₂₂₃ may each independently be a C₃-C₆₀ carbocyclic group or a C₁-C₆₀ heterocyclic group, each substituted with a cyano group—F;—Cl; —Br;—I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Non-limiting examples of the metal may be an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Non-limiting examples of the metalloid may be silicon (Si), antimony (Sb), and/or tellurium (Te).

Non-limiting examples of the non-metal may be oxygen (O) and/or a halogen (for example, F, Cl, Br, I, etc.).

Non-limiting examples of the compound including element EL1 and element EL2 may be metal oxides, metal halides (for example, metal fluorides, metal chlorides, metal bromides, or metal iodides), metalloid halides (for example, metalloid fluorides, metalloid chlorides, metalloid bromides, or metalloid iodides), metal tellurides, or one or more combinations thereof.

Non-limiting examples of the metal oxide may be tungsten oxides (for example, WO, W₂O₃, WO₂, WO₃, W₂O₅, etc.), vanadium oxides (for example, VO, V₂O₃, VO₂, V₂O₅, etc.), molybdenum oxides (MoO, Mo₂O₃, MoO₂, MoO₃, Mo₂O₅, etc.), and/or rhenium oxides (for example, ReO₃, etc.).

Non-limiting examples of the metal halide may be alkali metal halides, alkaline earth metal halides, transition metal halides, post-transition metal halides, and/or lanthanide metal halides.

Non-limiting examples of the alkali metal halide may be LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Non-limiting examples of the alkaline earth metal halide may be BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, Sri₂, and/or BaI₂.

Non-limiting examples of the transition metal halide may be titanium halides (for example, TiF₄, TiCl₄, TiBr₄, TiI₄, etc.), zirconium halides (for example, ZrF₄, ZrCl₄, ZrBr₄, ZrI₄, etc.), hafnium halides (for example, HfF₄, HfCl₄, HfBr₄, HfI₄, etc.), vanadium halides (for example, VF₃, VCl₃, VBr₃, VI₃, etc.), niobium halides (for example, NbF₃, NbCl₃, NbBr₃, NbI₃, etc.), tantalum halides (for example, TaF₃, TaCl₃, TaBr₃, TaI₃, etc.), chromium halides (for example, CrF₃, CrCl₃, CrBr₃, CrI₃, etc.), molybdenum halides (for example, MoF₃, MoCl₃, MoBr₃, MoI₃, etc.), tungsten halides (for example, WF₃, WCl₃, WBr₃, WI₃, etc.), manganese halides (for example, MnF₂, MnCl₂, MnBr₂, MnI₂, etc.), technetium halides (for example, TcF₂, TcCl₂, TcBr₂, TcI₂, etc.), rhenium halides (for example, ReF₂, ReCl₂, ReBr₂, ReI₂, etc.), ferrous halides (for example, FeF₂, FeCl₂, FeBr₂, FeI₂, etc.), ruthenium halides (for example, RuF₂, RuCl₂, RuBr₂, RuI₂, etc.), osmium halides (for example, OsF₂, OsCl₂, OsBr₂, OsI₂, etc.), cobalt halides (for example, CoF₂, CoCl₂, CoBr₂, CoI₂, etc.), rhodium halides (for example, RhF₂, RhCl₂, RhBr₂, RhI₂, etc.), iridium halides (for example, IrF₂, IrCl₂, IrBr₂, IrI₂, etc.), nickel halides (for example, NiF₂, NiCl₂, NiBr₂, NiI₂, etc.), palladium halides (for example, PdF₂, PdCl₂, PdBr₂, PdI₂, etc.), platinum halides (for example, PtF₂, PtCl₂, PtBr₂, PtI₂, etc.), cuprous halides (for example, CuF, CuCl, CuBr, CuI, etc.), silver halides (for example, AgF, AgCl, AgBr, AgI, etc.), and/or gold halides (for example, AuF, AuCl, AuBr, AuI, etc.).

Non-limiting examples of the post-transition metal halide may be zinc halides (for example, ZnF₂, ZnCl₂, ZnBr₂, ZnI₂, etc.), indium halides (for example, InI₃, etc.), and/or tin halides (for example, SnI₂, etc.).

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide may be antimony halides (for example, SbCls and/or the like) and/or the like.

Non-limiting examples of the metal telluride may be alkali metal tellurides (for example, Li₂Te, Na₂Te, K₂Te, Rb₂Te, Cs₂Te, etc.), alkaline earth metal tellurides (for example, BeTe, MgTe, CaTe, SrTe, BaTe, etc.), transition metal tellurides (for example, TiTe₂, ZrTe₂, HfTe₂, V₂Te₃, Nb₂Te₃, Ta₂Te₃, Cr₂Te₃, MozTe₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal tellurides (for example, ZnTe, etc.), and/or lanthanide metal tellurides (for example, LaTe, CeTe, PrTe, NdTe, PmTe, EuTe, GdTe, TbTe, DyTe, HoTe, ErTe, TmTe, YbTe, LuTe, etc.)

In one or more embodiments, an amount of the p-dopant in the activation layer 140 may be in a range of about 0.1 vol % to about 10 vol %, for example, about 0.5 vol % to about 5 vol %, based a total volume of the activation layer.

In one or more embodiments, the activation layer 140 may have a thickness of about 200 Å to about 2,000 Å, for example, about 400 Å to about 600 Å.

In one or more embodiments, the activation layer may absorb green light and/or red light, and activation layer may absorb light of near-infrared region.

In one or more embodiments, the first electrode may be an anode, the second electrode may be a cathode, a hole transport region may be further provided between the activation layer and the first electrode, and the hole transport region may include a hole injection layer, a hole transport layer, an auxiliary layer, an electron blocking layer, or any combination thereof.

In one or more embodiments, the first electrode may be an anode, the second electrode may be a cathode, an electron transport region may be further provided between the activation layer and the second electrode, and the electron transport region may include a buffer layer, a hole blocking layer, an electron transport layer, an electron injection layer, or any combination thereof.

### First Electrode 110

In FIG. 1, in one or more embodiments, a substrate may be additionally provided and arranged under the first electrode 110 and/or a substrate may be additionally provided and arranged on the second electrode 170. As the substrate, a glass substrate or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, the material for forming the first electrode 110 may be a high-work function material.

The first electrode 110 may be a reflective electrode, a semi-transmissive electrode, or a transmissive electrode. In one or more embodiments, when the first electrode 110 is a transmissive electrode, indium tin oxide (ITO), indium zinc oxide (IZO), tin oxide (SnO₂), zinc oxide (ZnO), or any combination thereof may be utilized as a material for forming the first electrode 110. In one or more embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combinations thereof may be utilized as a material for forming the first electrode 110.

The first electrode 110 may have a single-layered structure including (e.g., consisting of) a single layer or a multi-layered structure including a plurality of layers. For example, in some embodiments, the first electrode 110 may have a three-layered structure of ITO/Ag/ITO.

### Charge Auxiliary Layer

The organic photodetector 10 according to one or more embodiments may include a charge auxiliary layer that facilitates migration of holes and electrons from the activation layer 140.

The charge auxiliary layer may include the hole injection layer 120 and the hole transport layer 130, which facilitate migration of holes, and may include the electron transport layer 150 and the electron injection layer 160, which facilitate migration of electrons.

### Hole Transport Region

The charge auxiliary layers located between the first electrode 110 and the activation layer 140 may be collectively referred to as a hole transport region.

In one or more embodiments, the hole transport region may include the hole injection layer 120, the hole transport layer 130, an auxiliary layer, and an electron blocking layer.

The hole transport region may include a hole transport material. For example, in one or more embodiments, the hole transport material may include a compound represented by Formula 201, a compound represented by Formula 202, or any combination thereof:

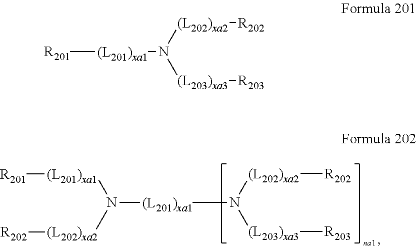

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—** , *—S—** , *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, see Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, in some embodiments, each of Formulae 201 and 202 may include at least one selected from groups represented by Formulae CY201 to CY217:

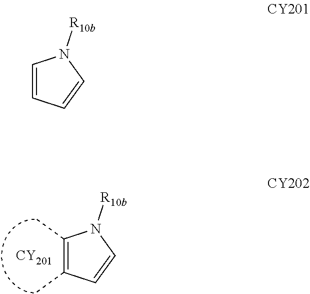

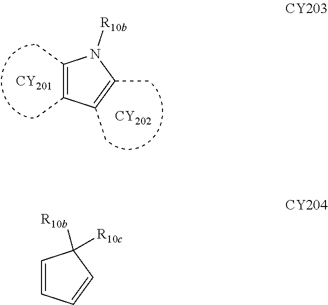

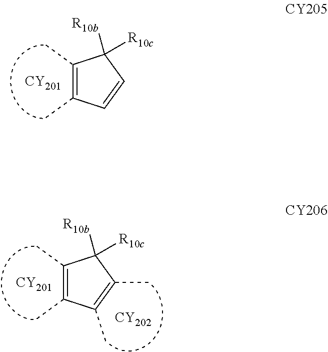

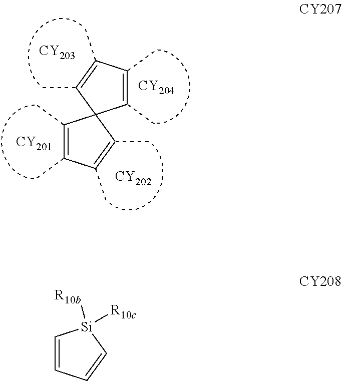

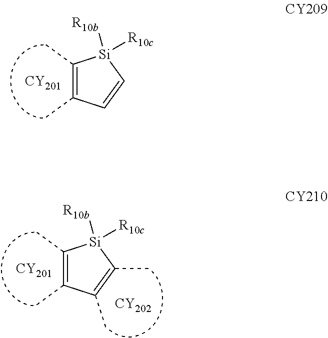

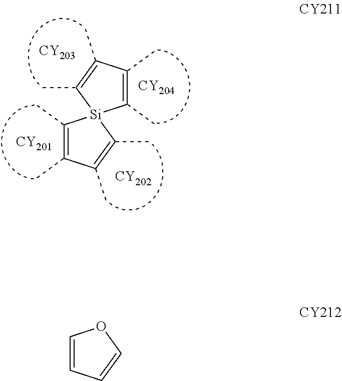

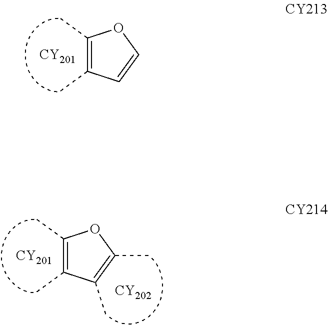

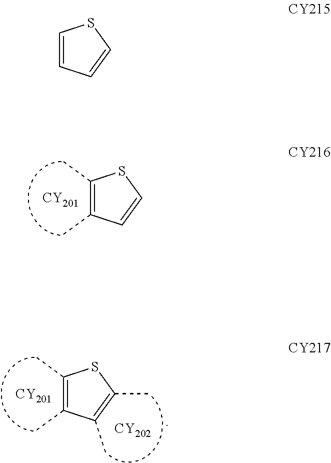

In Formulae CY201 to CY217, R_{10b} and R_{10c} may each be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include (e.g., may exclude) any group represented by one selected from Formulae CY201 to CY203.

In one or more embodiments, each of Formulae 201 and 202 may not include (e.g., may exclude) any group represented by one selected from Formulae CY201 to CY203, and may include at least one selected from the groups represented by Formulae CY204 to CY217.

In one or more embodiments, each of Formulae 201 and 202 may not include (e.g., may exclude) any group represented by one selected from Formulae CY201 to CY217. In present disclosure, “not include a or any ‘component’” “exclude a or any ‘component’”, “‘component’-free”, and/or the like refers to that the “component” not being added, selected or utilized as a component in the composition/element, but, in some embodiments, the “component” of less than a suitable amount may still be included due to other impurities and/or external factors.

For example, in one or more embodiments, the hole transport material may

include at least one selected from among Compounds HT1 to HT46, 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB(NPD)), B-NPB, N,N′-bis(3-methylphenyl)-N,N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

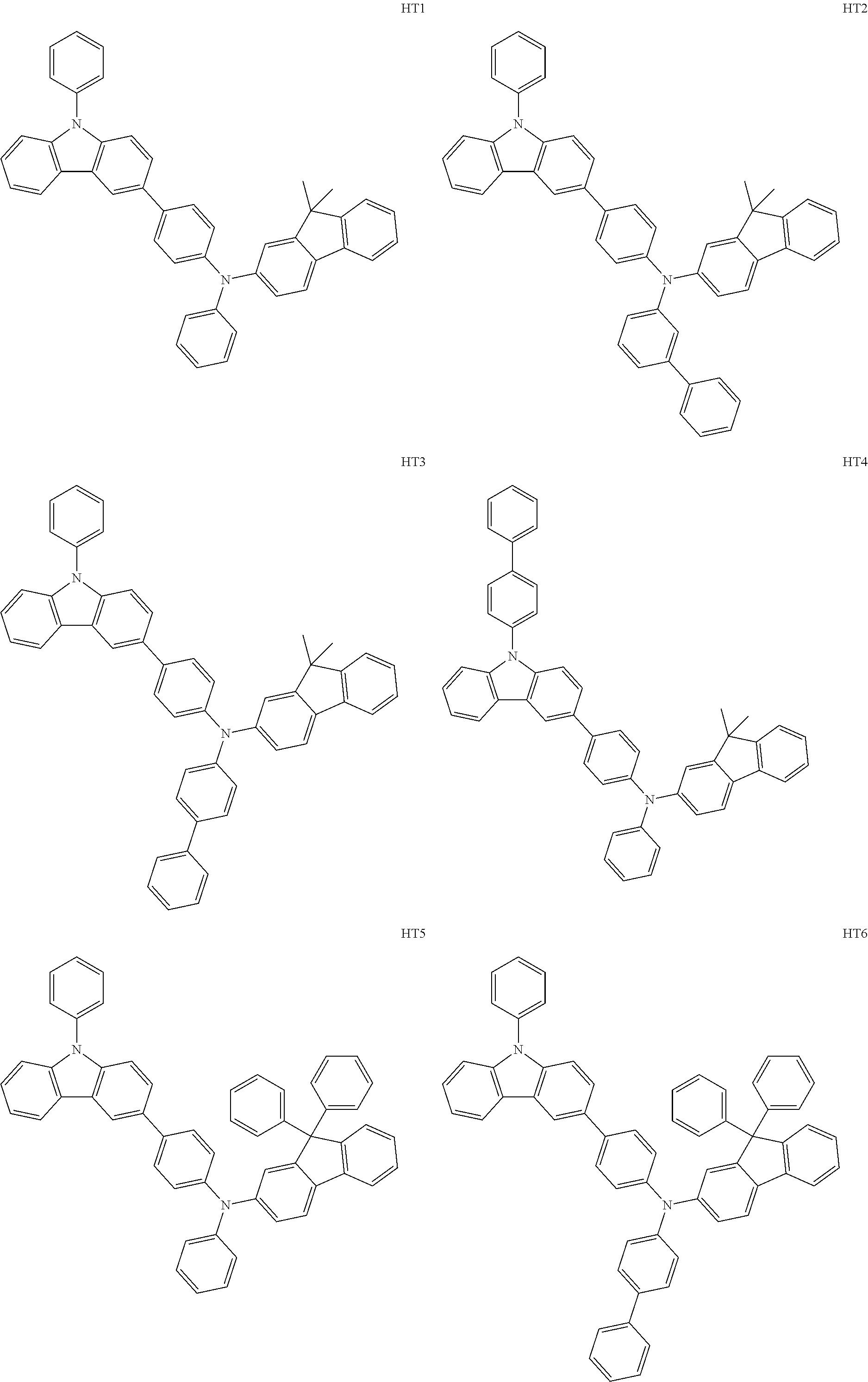

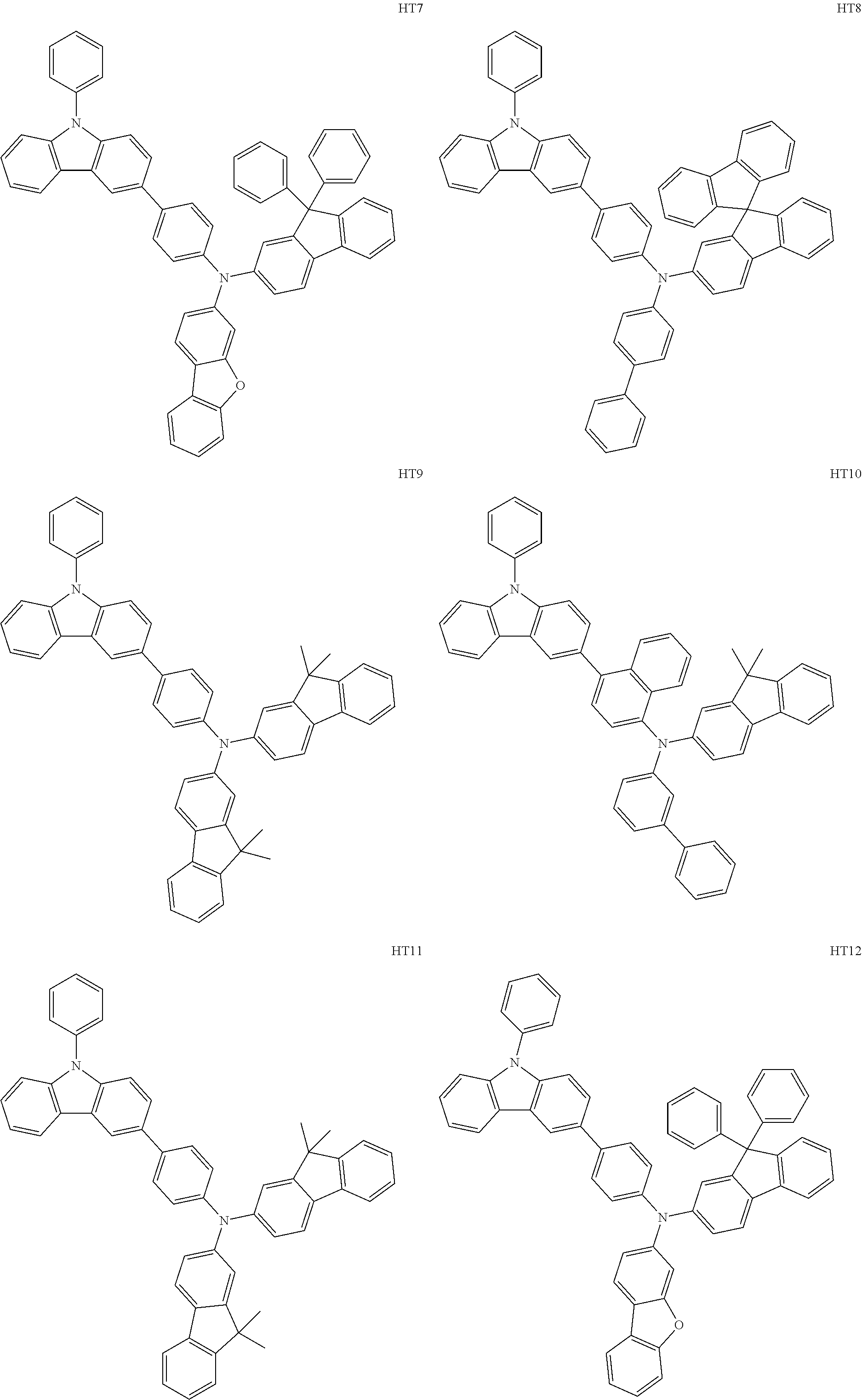

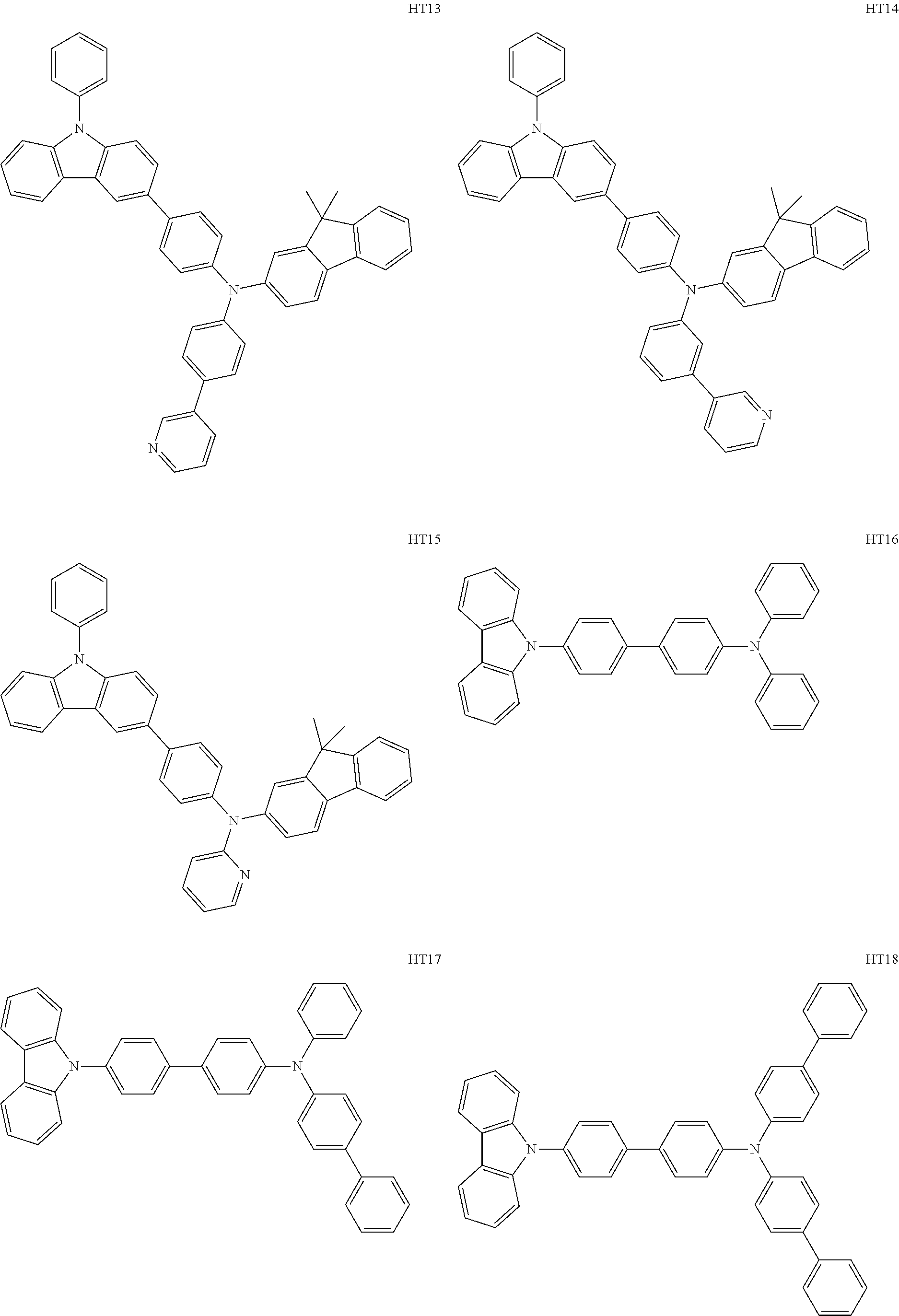 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

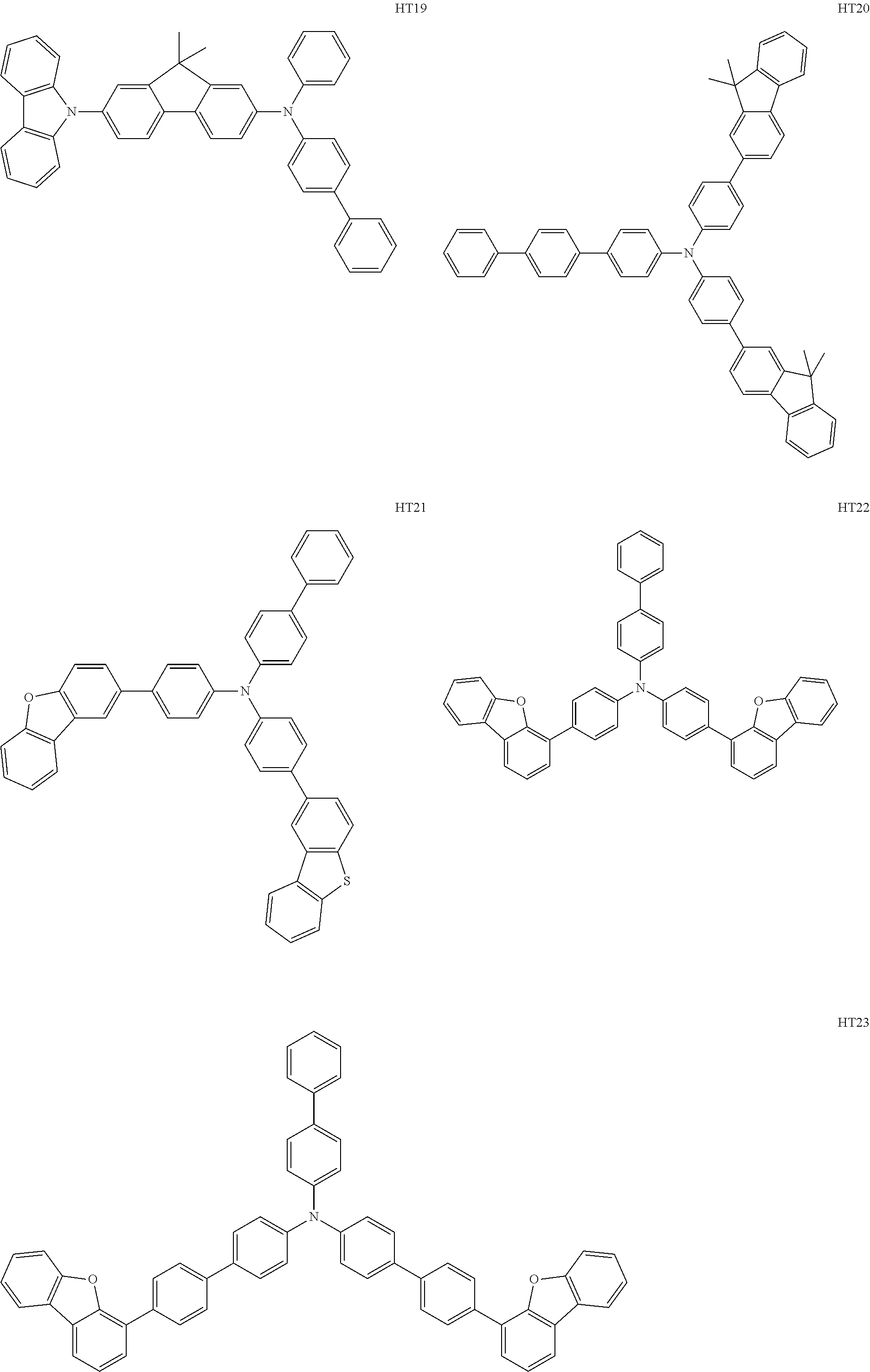 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

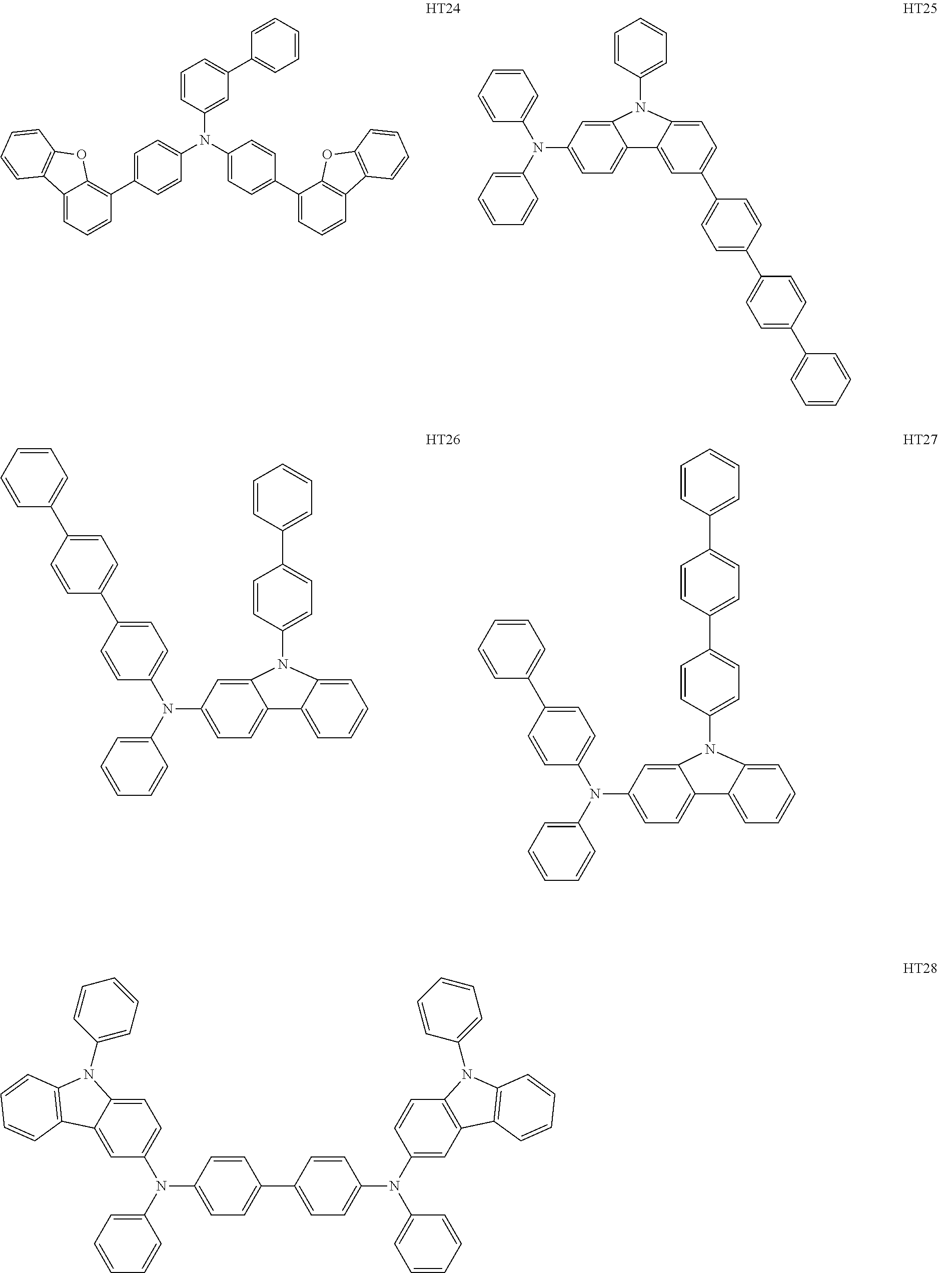 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

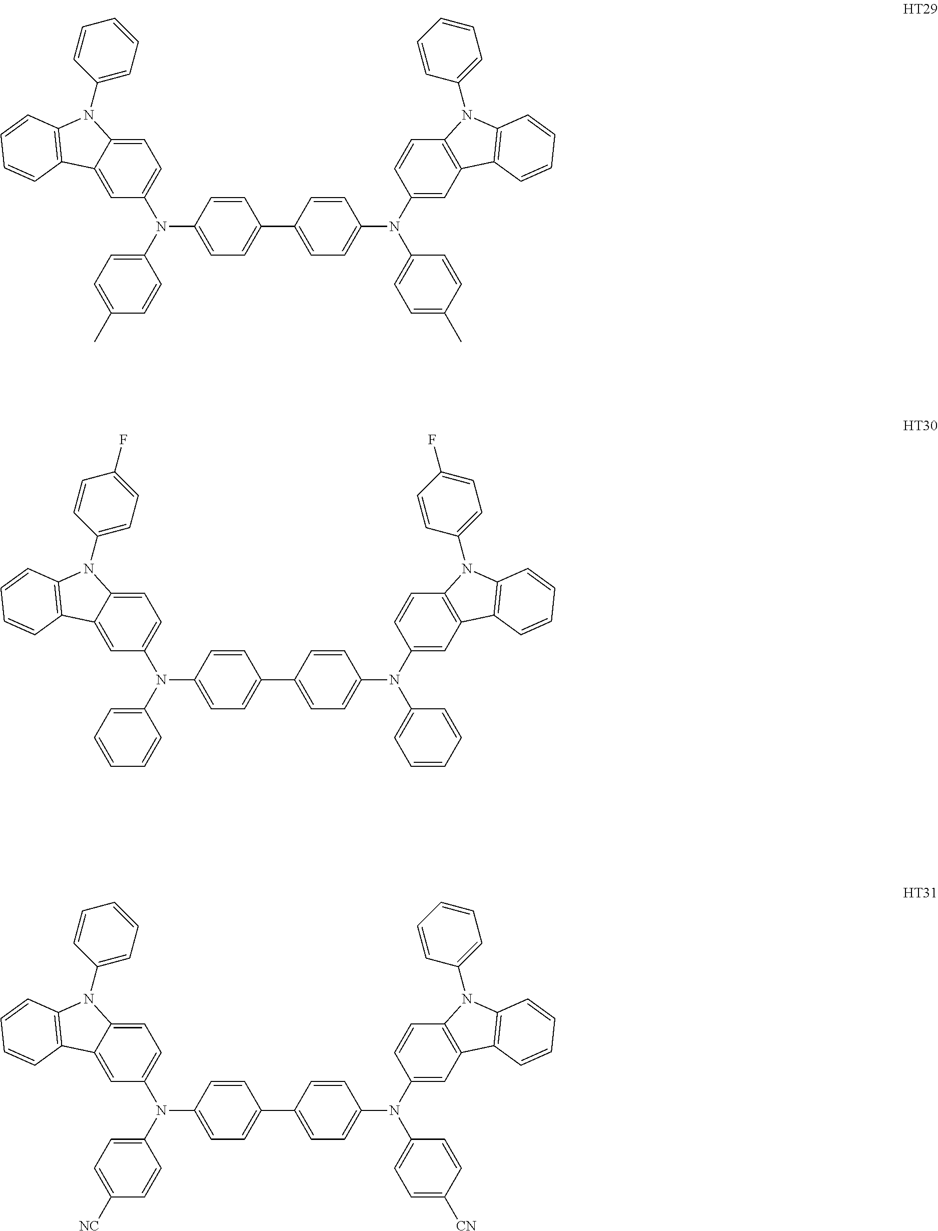 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

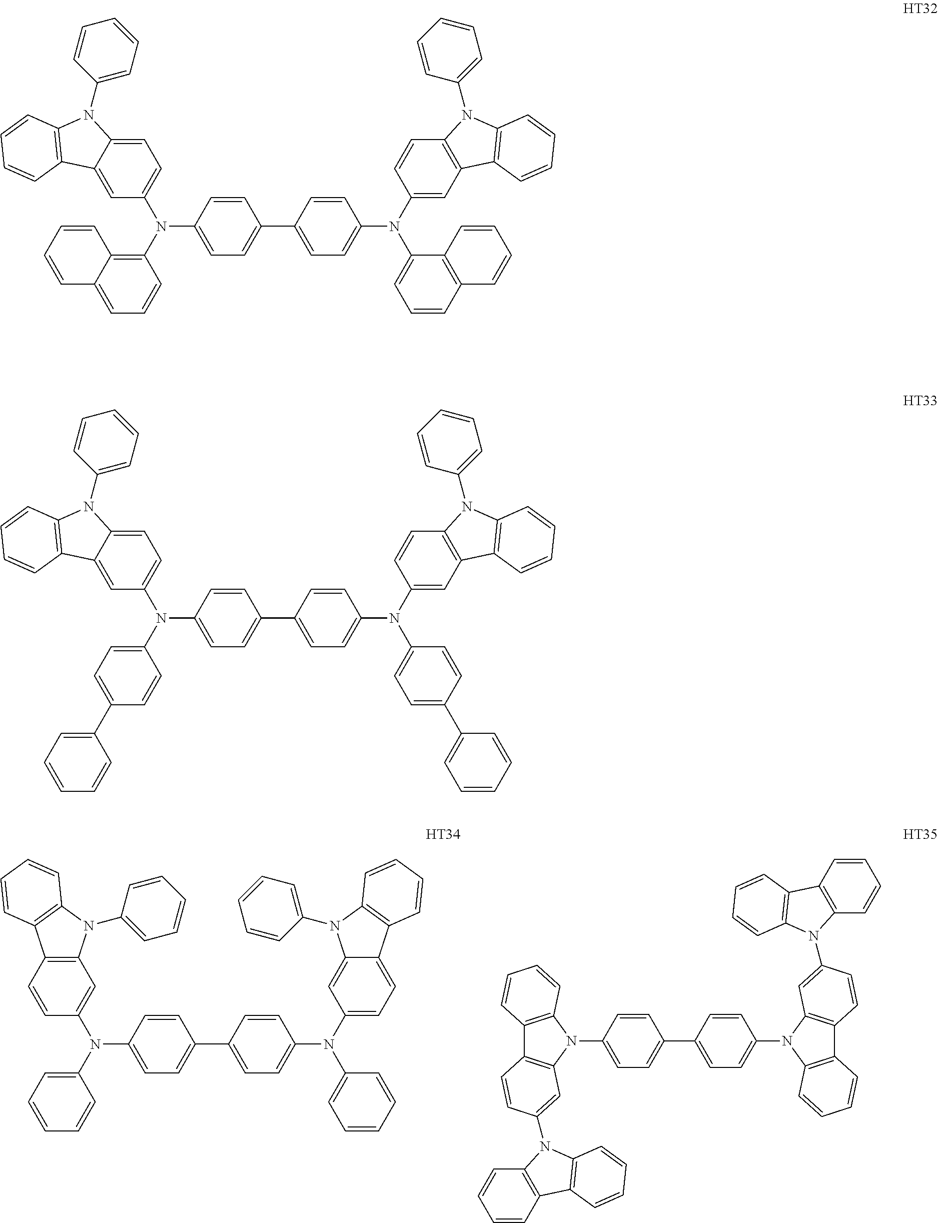 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

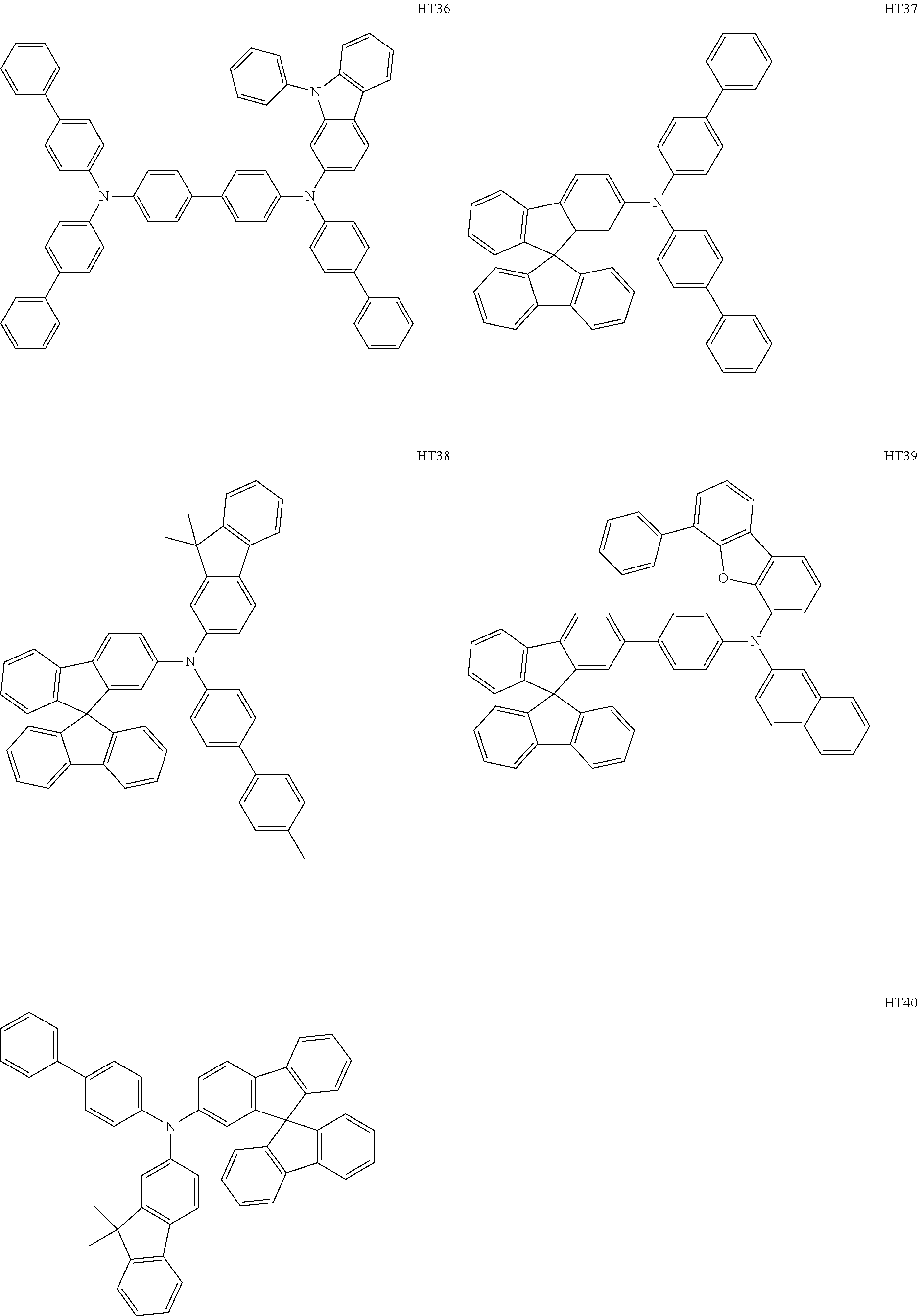

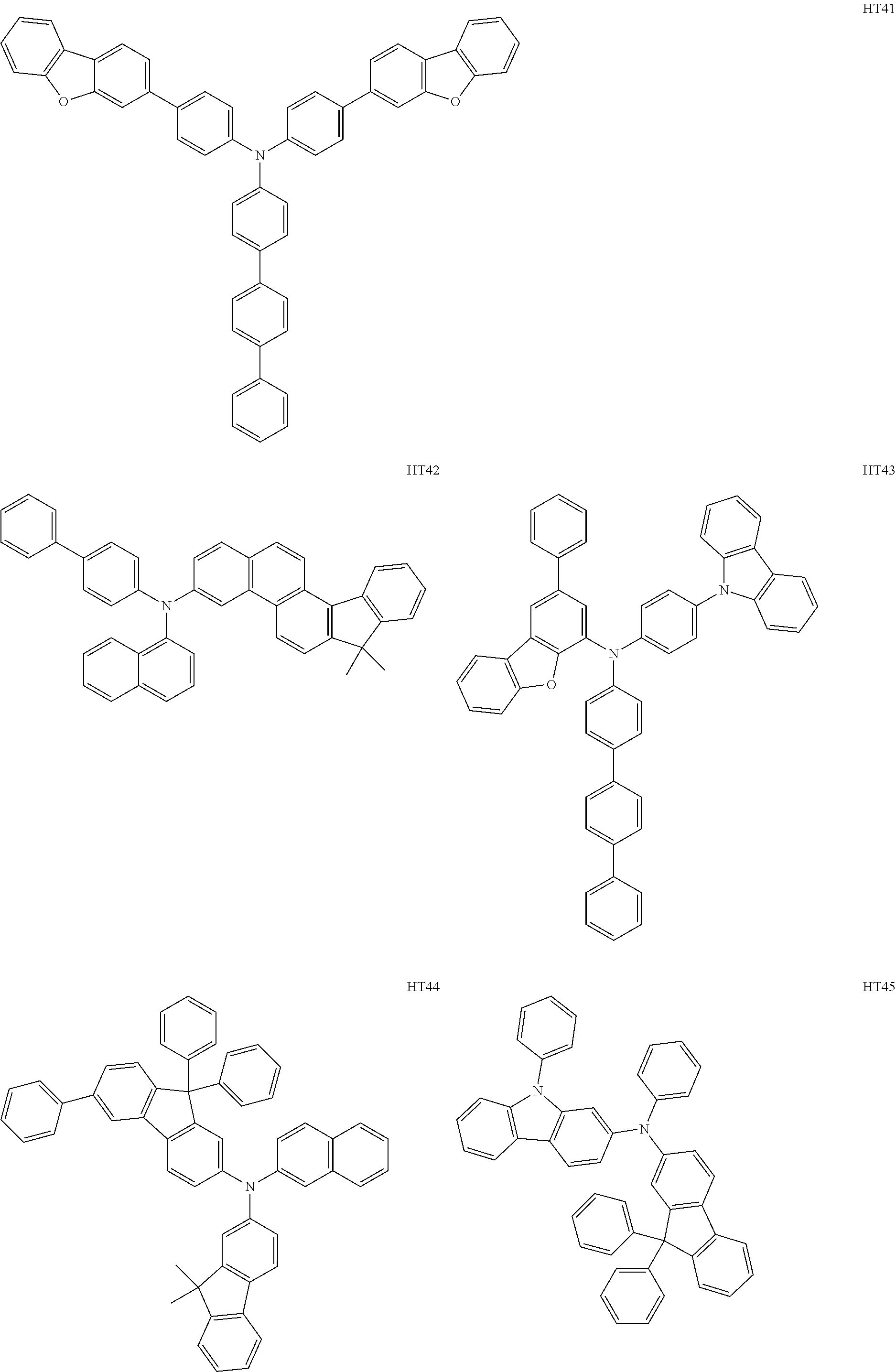 [SMILES: c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1]

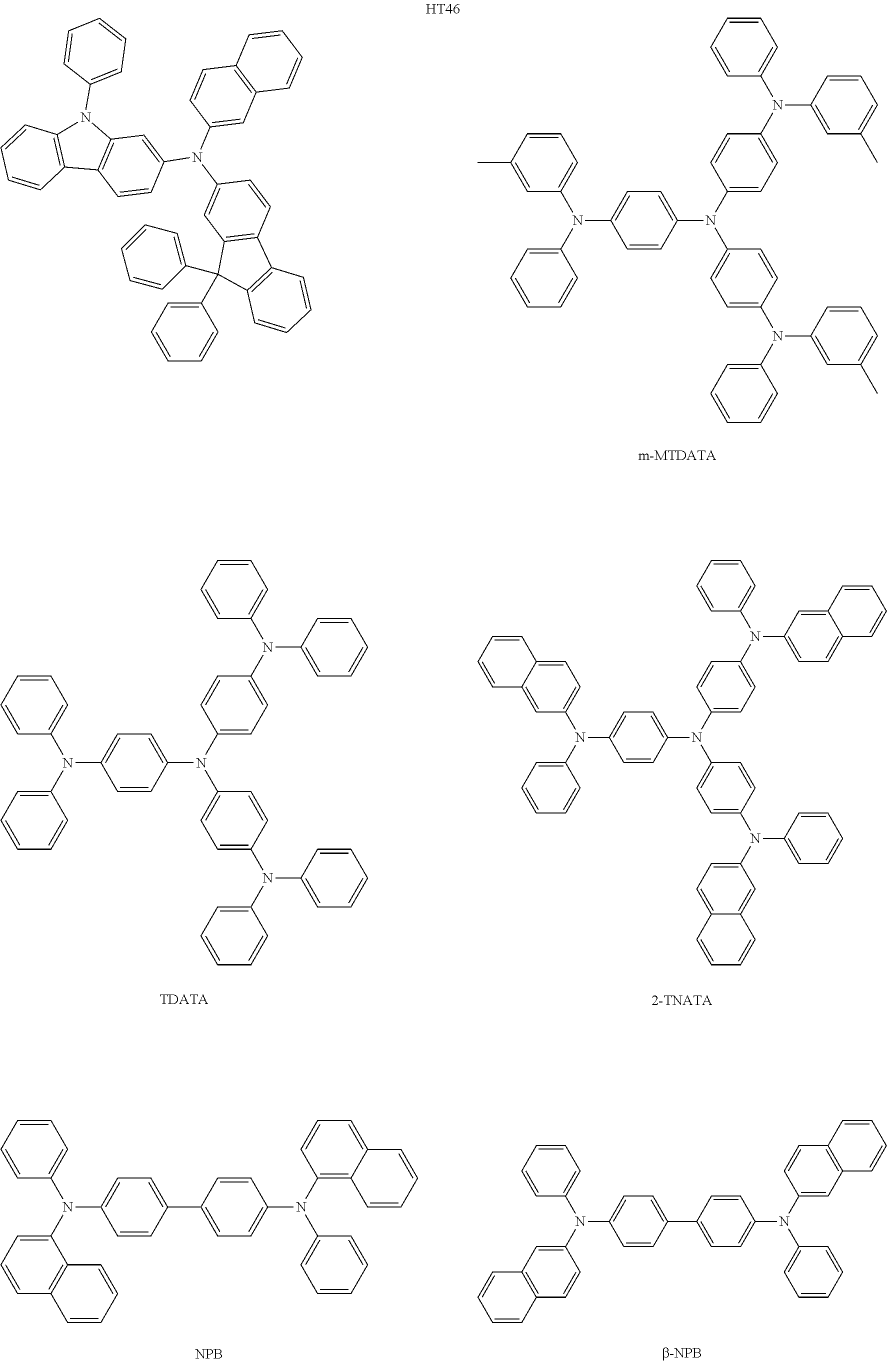

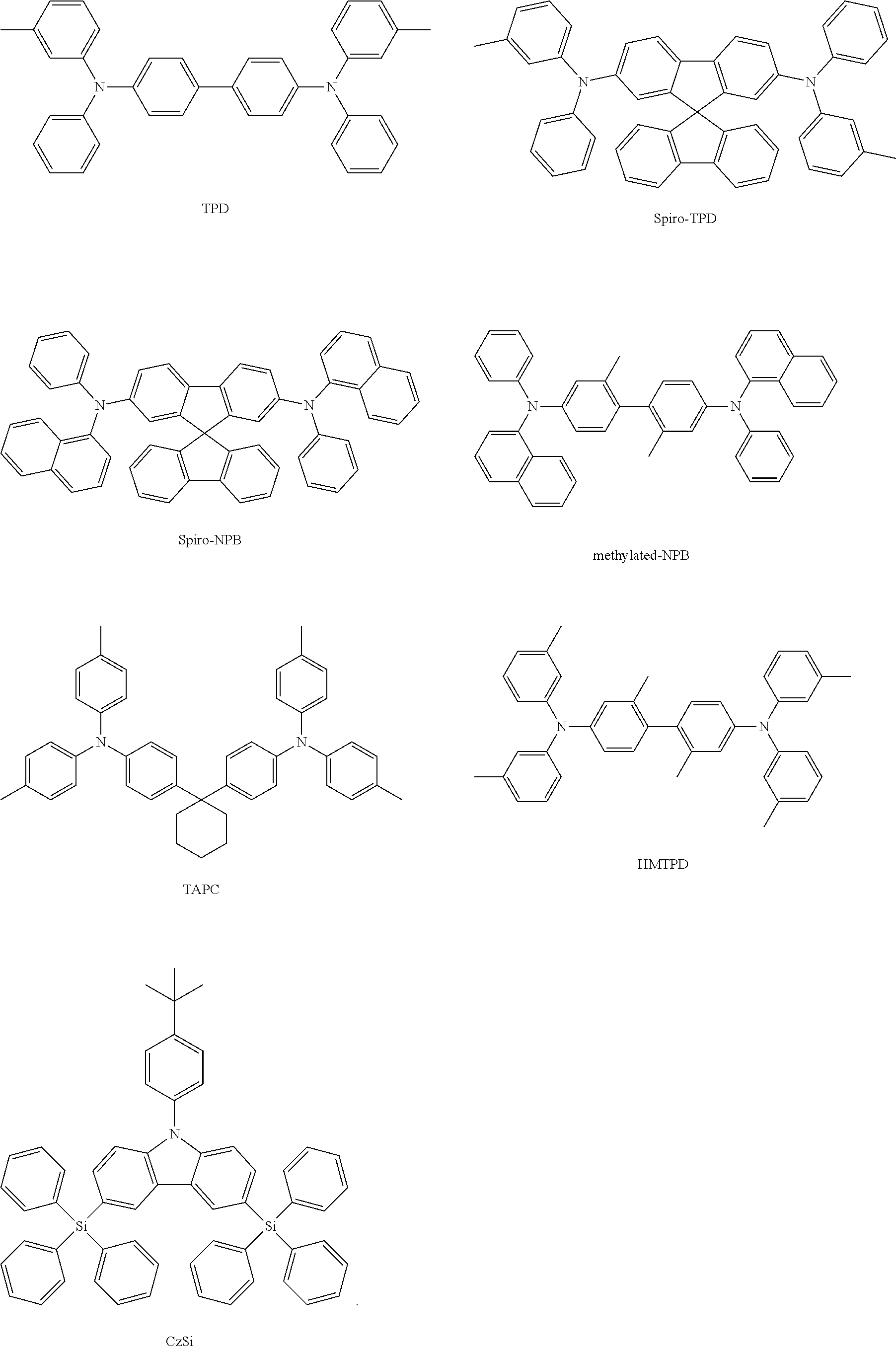

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The auxiliary layer may compensate for an optical resonance distance according to a wavelength of light introduced to the activation layer 140 to increase light introduction efficiency. The electron blocking layer serves to prevent leakage of electrons from the activation layer 140 into the hole transport region. The hole transport material may be included in the auxiliary layer and the electron blocking layer.

### Electron Transport Region

The charge auxiliary layers located between the activation layer 140 and the second electrode 170 may be collectively referred to as an electron transport region.

The electron transport region may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron transport region may include a buffer layer, a hole blocking layer, the electron transport layer 150, the electron injection layer 160, or any combination thereof.

For example, in one or more embodiments, the electron transport region may have an electron transport layer/electron injection layer structure, a hole blocking layer/electron transport layer/electron injection layer structure, or a buffer layer/electron transport layer/electron injection layer structure, wherein the constituent layers of each structure are stacked sequentially from the emission layer in the stated order.

In one or more embodiments, the electron transport region (for example, the buffer layer, the hole blocking layer, or the electron transport layer in the electron transport region) may include a metal-free compound including at least one π electron-deficient nitrogen-containing C₁-C₆₀ cyclic group.

For example, in one or more embodiments, the electron transport region may include a compound represented by Formula 601:

[Ar₆₀₁]ₓₑ₁₁-[(L₆₀₁)ₓₑ₁-R₆₀₁]ₓₑ₂₁, Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁k)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each be the same as described herein with respect to Q₁, xe21 may be 1, 2, 3, 4, or 5, and

at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In some embodiments, Ar₆₀₁ in Formula 601 may be a substituted or unsubstituted anthracene group.

In some embodiments, the electron transport region may include a compound represented by Formula 601-1:

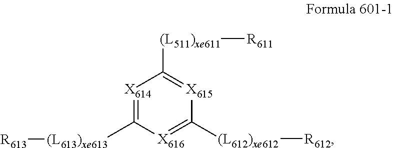

wherein, in Formula 601-1,

X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,

L₆₁₁ to L₆₁₃ may each be the same as described herein with respect to L₆₀₁,

xe611 to xe613 may each be the same as described herein with respect to xe1,

R₆₁₁ to R₆₁₃ may each be the same as described herein with respect to R₆₀₁, and

R₆₁₄ to R₆₁₆ may each independently be hydrogen, deuterium, —F, —Cl, —Br, —I,

a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in some embodiments, xe1 and xe611 to xe613 in Formulae 601 and 601-1 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), tris(8-hydroxyquinolinato)aluminum (Alq₃), bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), diphenyl[4-(triphenylsilyl)phenyl]phosphine oxide (TSPO1), 1,3,5-tris(1-phenyl-1H-benzimidazol-2-yl)benzene (TPBI), or any combination thereof:

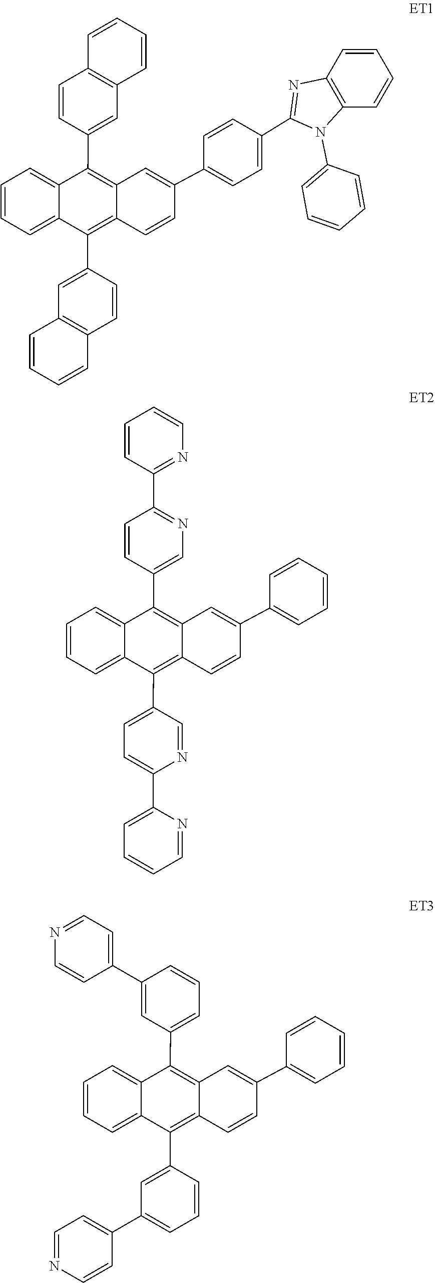 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

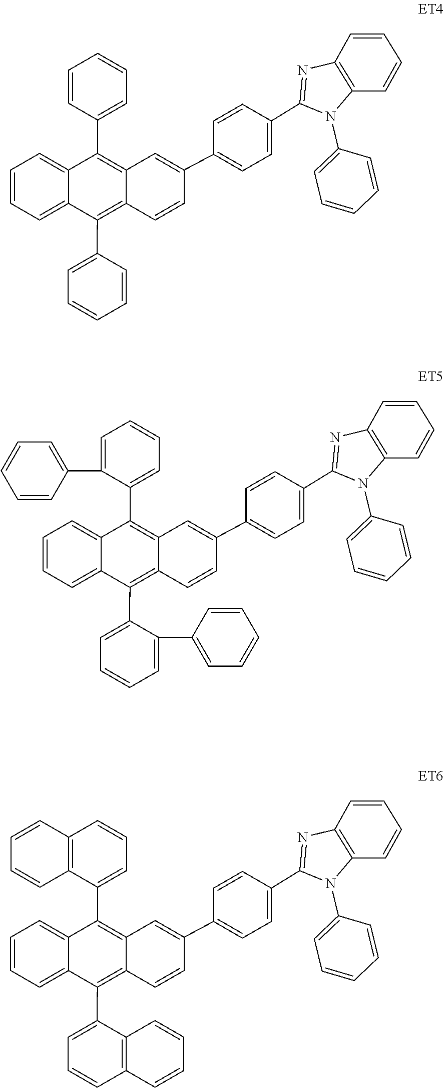 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

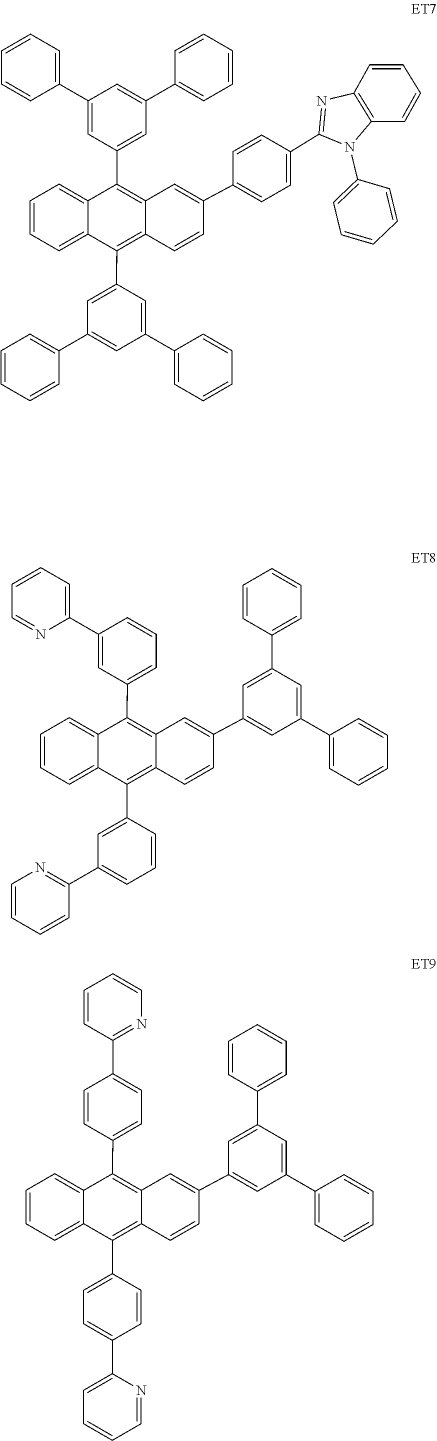 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

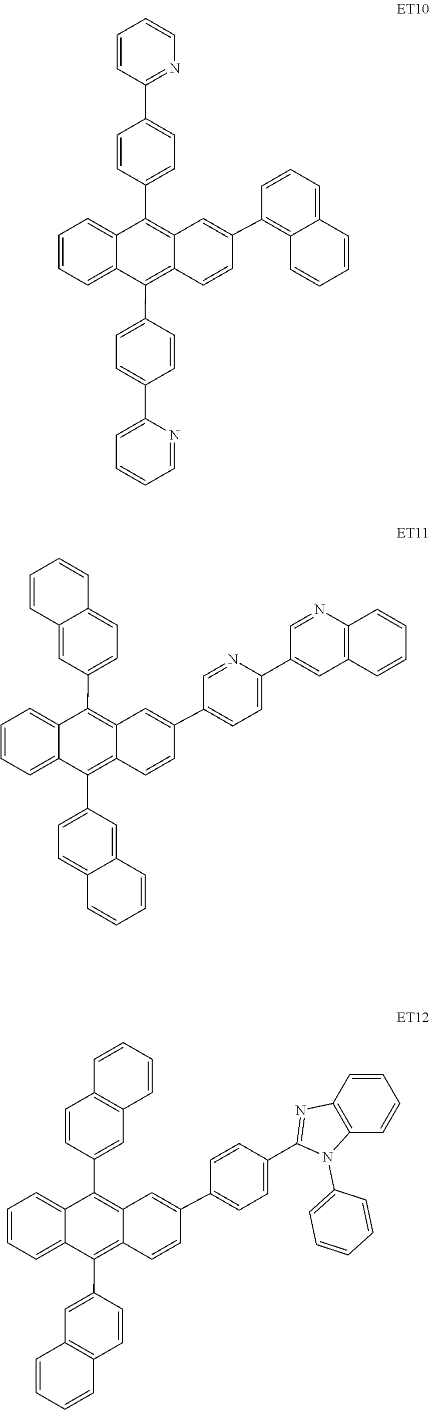 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

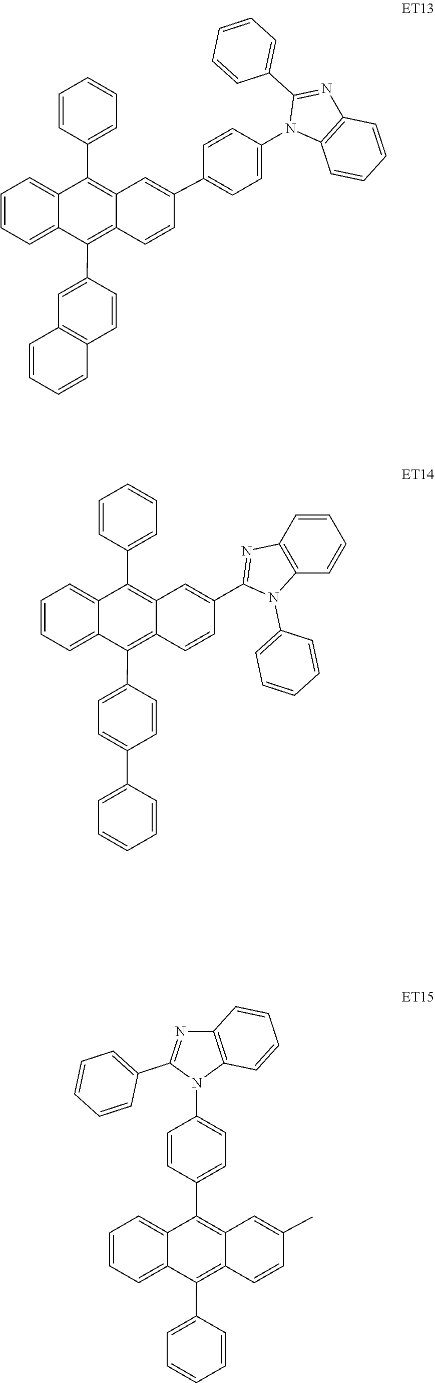 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

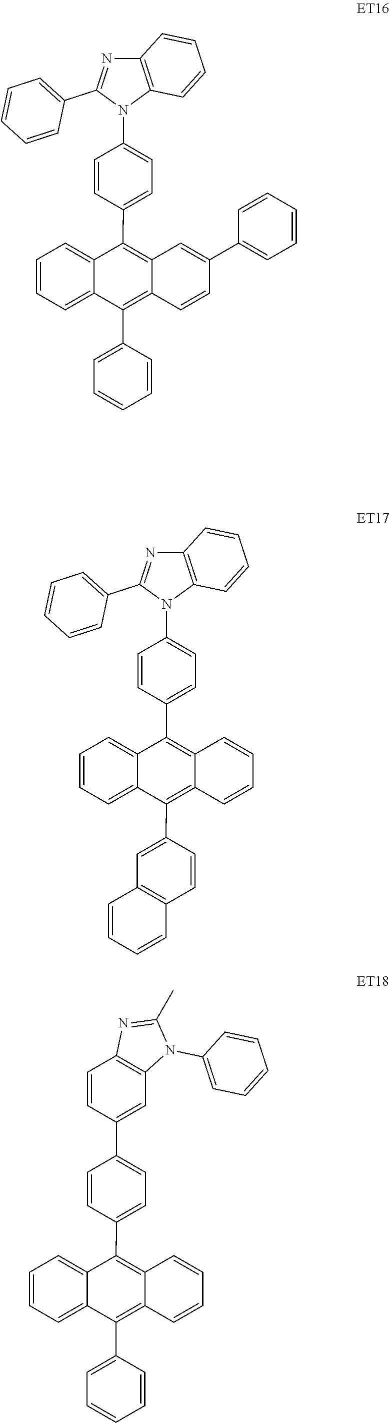 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

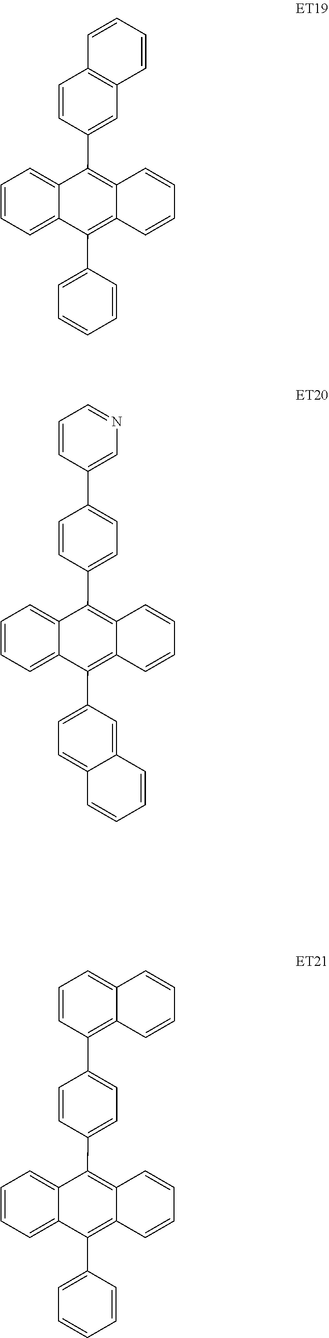

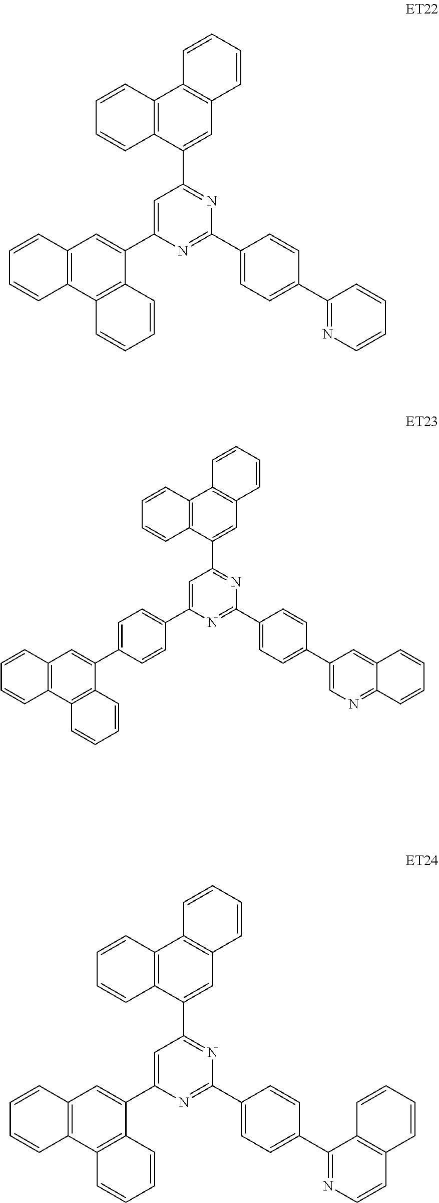 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

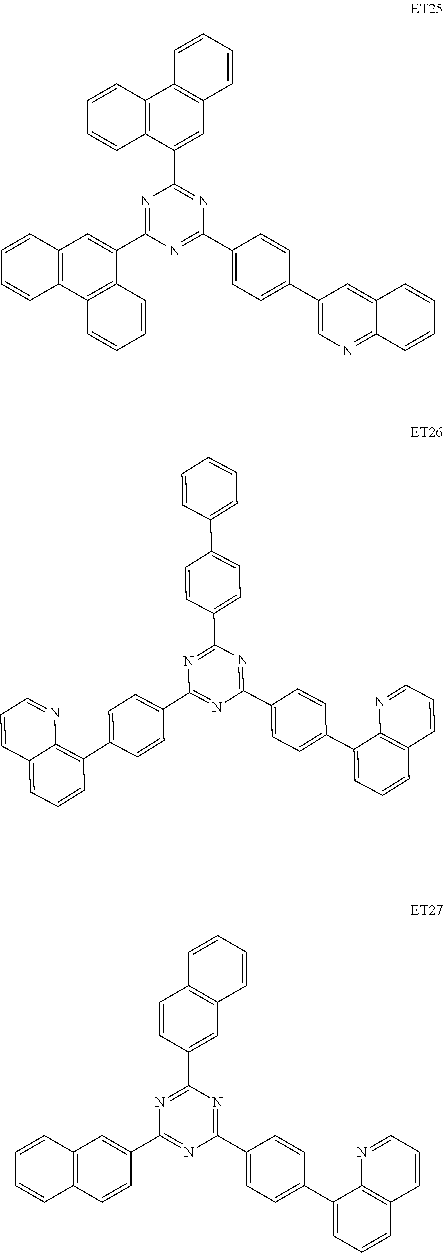 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

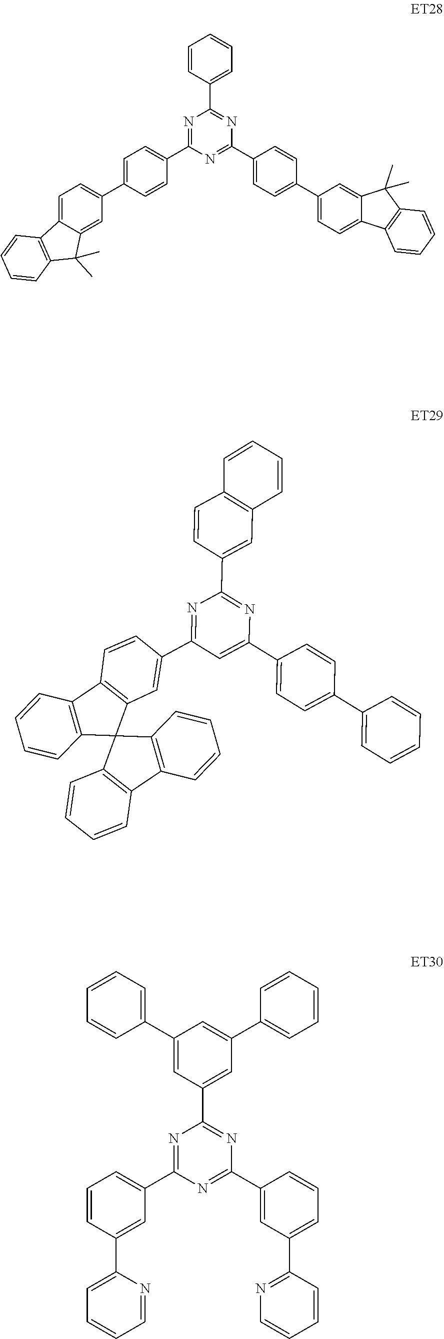 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

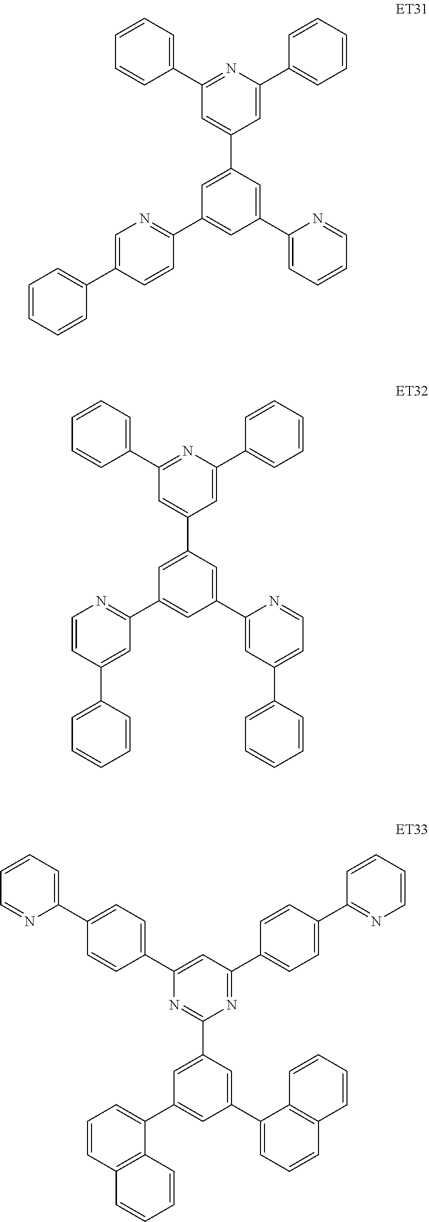 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

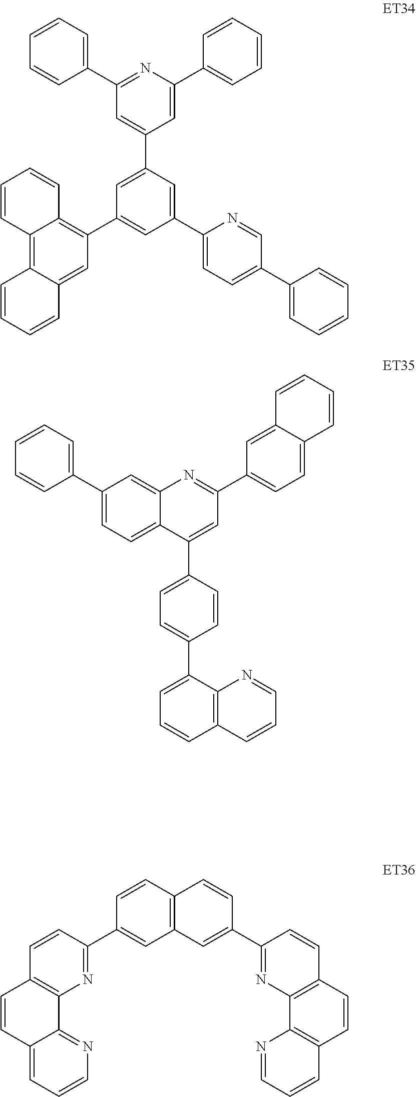

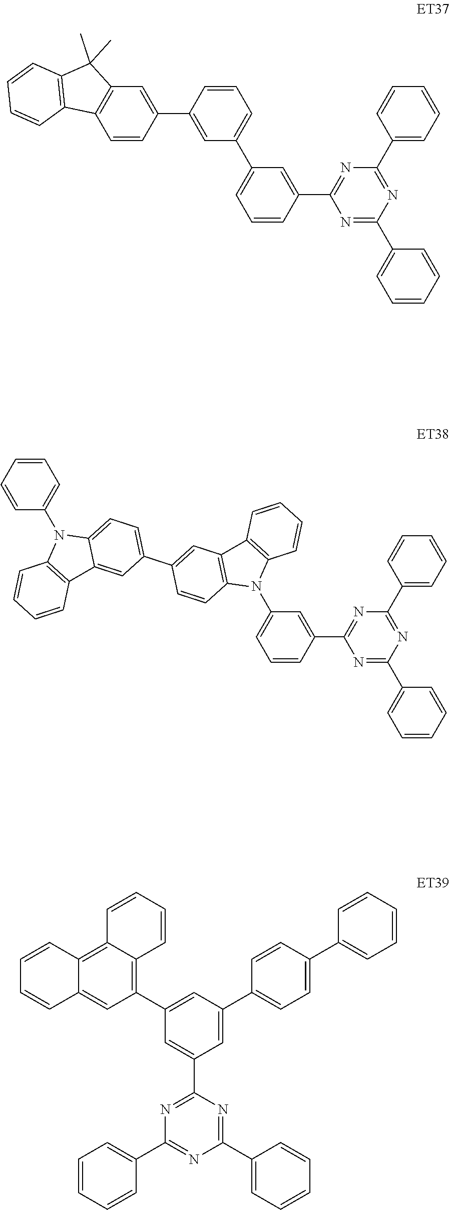 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

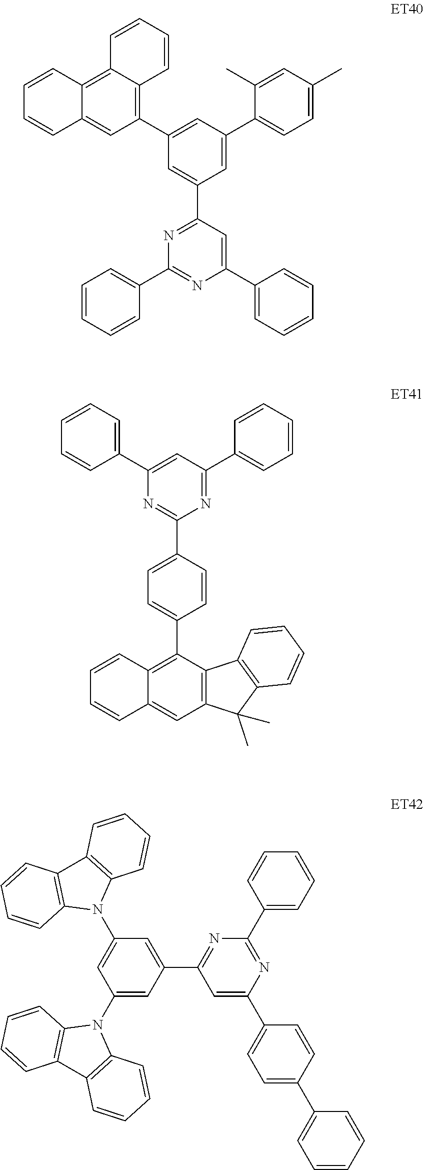 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

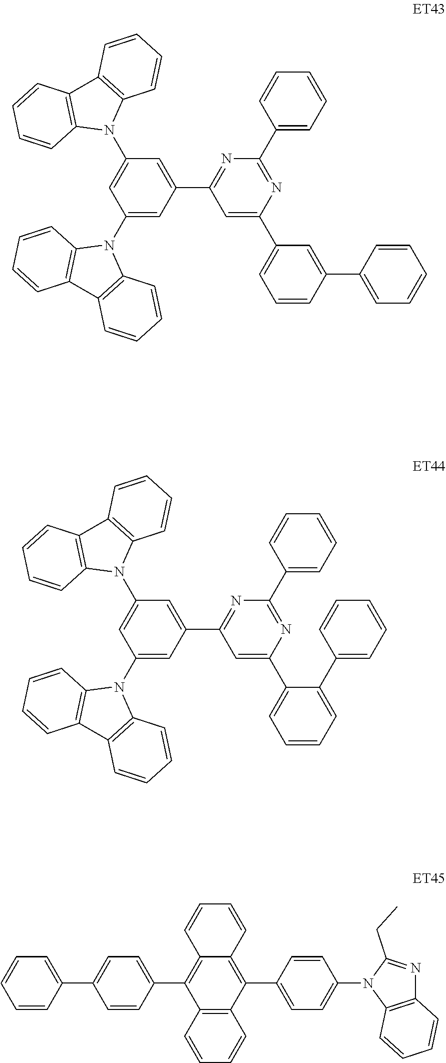 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

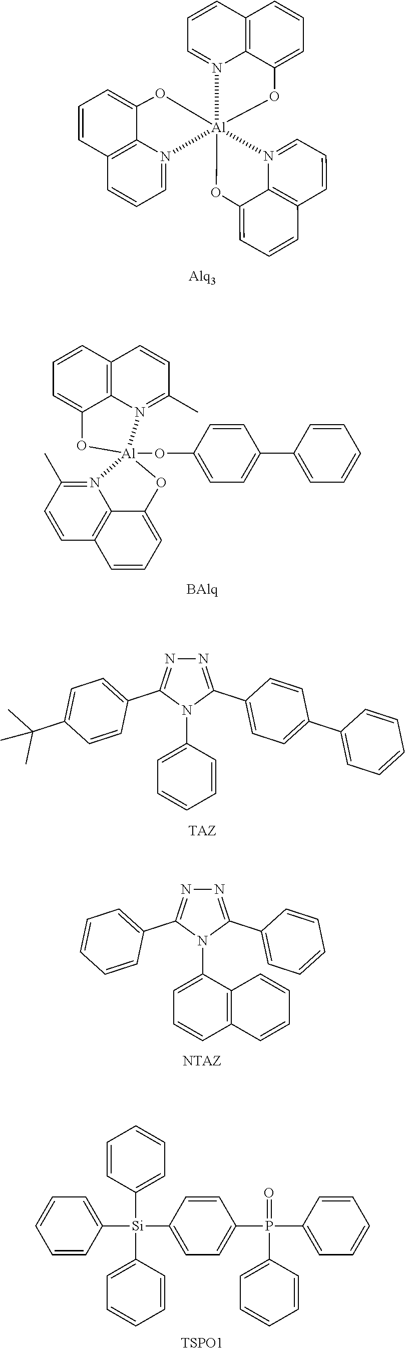

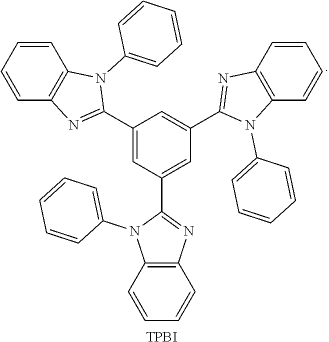

A thickness of the electron transport region may be from about 160 Å to about 5,000 Å, for example, from about 100 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be from about 20 Å to about 1,000 Å, for example, about 30 Å to about 300 Å, and a thickness of the electron transport layer may be from about 100 Å to about 1,000 Å, for example, about 150 Å to about 500 Å. When the thicknesses of the buffer layer, the hole-blocking layer, the electron control layer, the electron transport layer, and/or the electron transport layer are within these ranges, satisfactory electron transporting characteristics may be obtained without a substantial increase in driving voltage. In one or more embodiments, the electron transport region (for example, the

electron transport layer in the electron transport region) may further include, in addition to one or more of the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, in some embodiments, the metal-containing material may include a Li complex. The Li complex may include, for example, Compound ET-D1 (LiQ) or ET-D2:

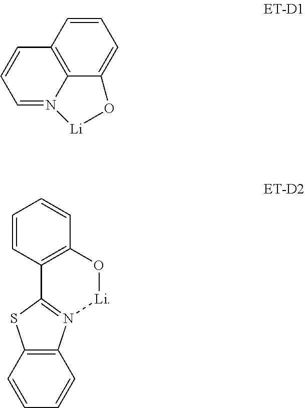

In one or more embodiments, the electron transport region may include an electron injection layer that facilitates the injection of electrons. The electron injection layer may directly contact the second electrode 170.

The electron injection layer may have: i) a single-layered structure including

(e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, respectively, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, SC₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal tellurides. Non-limiting examples of the lanthanide metal telluride may be LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one of metal ions of the alkali metal, one of the metal ions of the alkaline earth metal, and one of metal ions of the rare earth metal, respectively, and ii) a ligand bonded to the metal ion (i.e., the respective metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, in some embodiments, the electron injection layer may be a KI:Yb co-deposited layer, an Rbl:Yb co-deposited layer, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be substantially uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within the ranges described above, satisfactory electron injection characteristics may be obtained without a substantial increase in driving voltage.

### Second Electrode 170

The second electrode 170 may be located over the buffer layer 156 or the electron transport region as described above. The second electrode 190 may be a cathode, and as a material for the second electrode 190, a metal, an alloy, an electrically conductive compound, or any combination thereof, each having a low work function, may be utilized.

The second electrode 170 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 170 may be a transmissive electrode, a semi-transmissive electrode, or a reflective electrode.

The second electrode 170 may have a single-layered structure or a multi-layered structure including a plurality of layers.

Capping layer

A first capping layer may be located outside (e.g., on) the first electrode 110, and/or a second capping layer may be located outside (e.g., on) the second electrode 170.

The first capping layer and/or the second capping layer may prevent penetration of impurities, such as water and/or oxygen, to the organic photodetector to thereby improve reliability of the organic photodetector.

Each of the first capping layer and the second capping layer may include a material having a refractive index of 1.6 or more (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may (e.g., the first capping layer and the second capping layer may each independently) include carbocyclic compounds, heterocyclic compounds, amine group-containing compounds, porphine derivatives, phthalocyanine derivatives, a naphthalocyanine derivatives, alkali metal complexes, alkaline earth metal complexes, or any combination thereof. Optionally, the carbocyclic compound, the heterocyclic compound, and the amine group-containing compound may each be substituted with a substituent including O, N, S, Se, Si, F, Cl, Br, I, or any combination thereof. In one or more embodiments, at least one of the first capping layer or the second capping layer may (e.g., the first capping layer and the second capping layer may each independently) include an amine group-containing compound.

For example, in one or more embodiments, at least one of the first capping layer or the second capping layer may (e.g., the first capping layer and the second capping layer may each independently) include a compound represented by Formula 201, a compound represented by Formula 202, or any combination thereof.

In one or more embodiments, at least one of the first capping layer or the second capping layer may (e.g., the first capping layer and the second capping layer may each independently) include at least one selected from among Compounds HT28 to HT33, at least one selected from among Compounds CP1 to CP6, β-NPB, or any combination thereof:

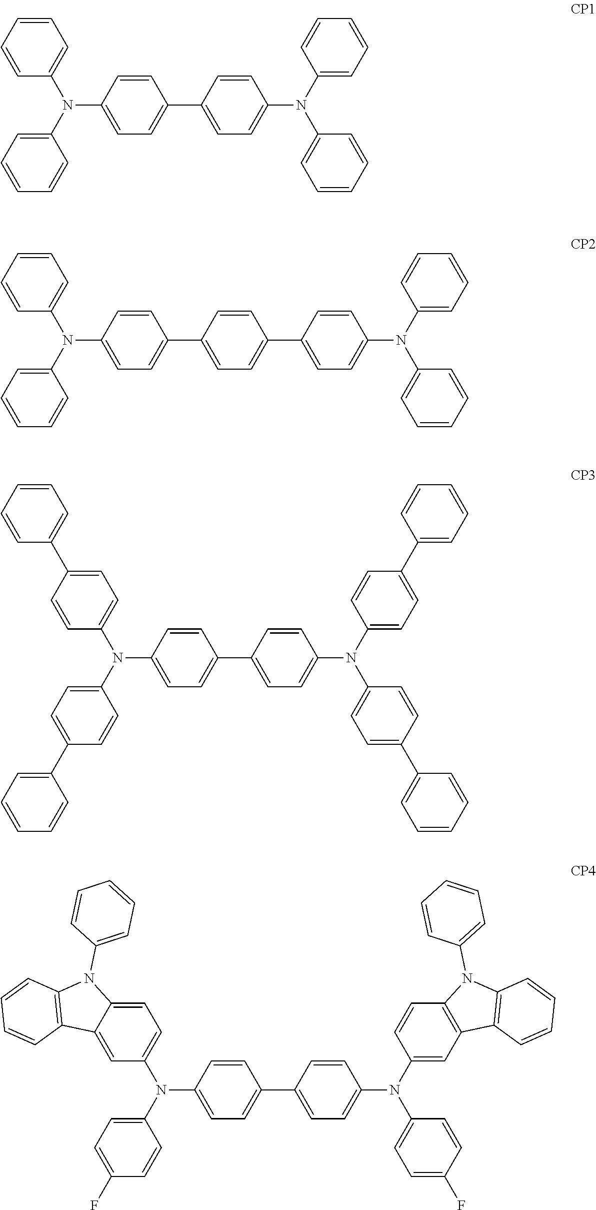 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

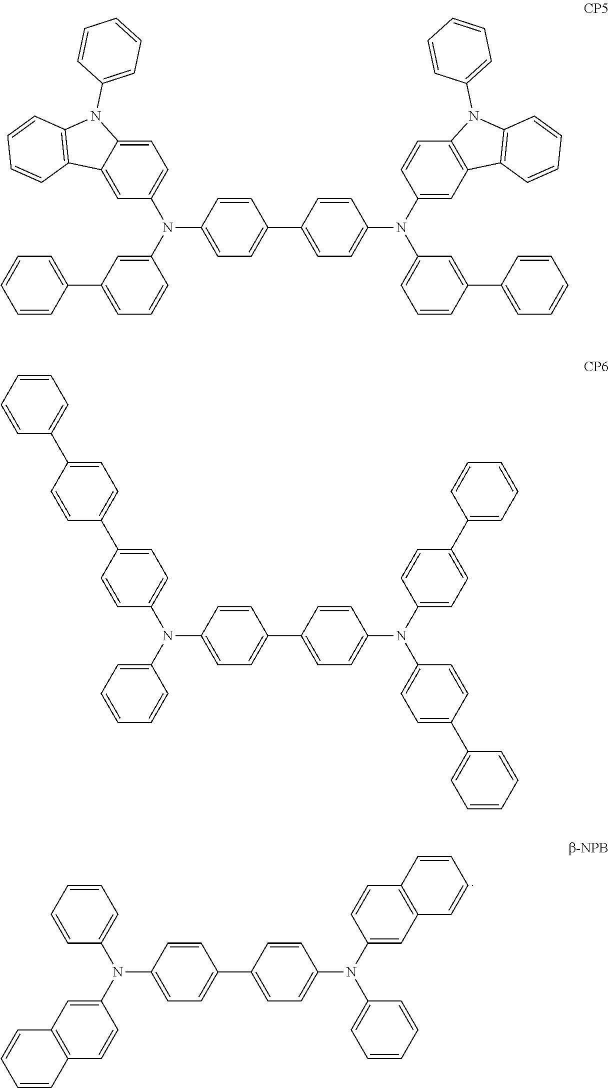 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

According to one or more embodiments of the present disclosure, provided is an electronic apparatus including the organic photodetector as described above. For example, in one or more embodiments, the electronic apparatus may further include a light-emitting device.

Accordingly, the electronic apparatus according to one or more embodiments may include: a substrate including a light detection region and an emission region;

an organic photodetector arranged on (e.g., in) the light detection region; and

a light-emitting device arranged on (e.g., in) the emission region,

wherein the organic photodetector may include: a first pixel electrode; a counter electrode facing the first pixel electrode; and a hole transport region, an activation layer, and an electron transport region, which are sequentially arranged between the first pixel electrode and the counter electrode,

the light-emitting device may include: a second pixel electrode; the counter electrode facing the second pixel electrode; and the hole transport region, an emission layer, and the electron transport region, which are sequentially arranged between the second pixel electrode and the counter electrode,

the first pixel electrode and the activation layer may be arranged in correspondence with the light detection region,

the second pixel electrode and the emission layer may be arranged in correspondence with the emission region, and the hole transport region, the electron transport region, and the counter

electrode may be arranged throughout the light detection region and the emission region.

The hole transport region and the electron transport region are respectively the same as described herein.

In one or more embodiments, the hole injection layer, the hole transport

layer, the electron transport layer, and/or the electron injection layer; and the counter electrode may be arranged throughout the light detection region and the emission region.

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

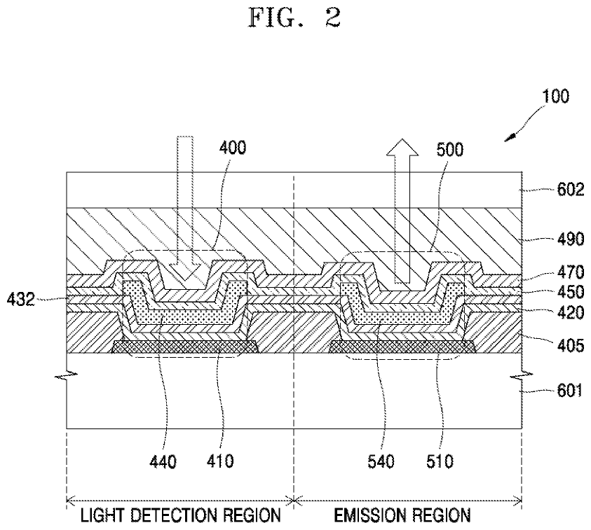

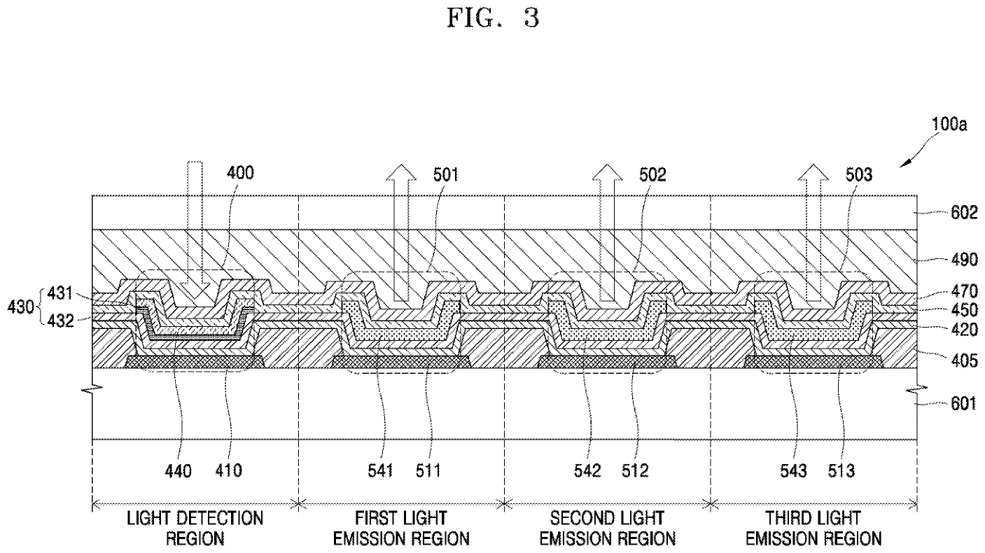

### Description of FIG. 2 and FIG. 3

FIG. 2 is a schematic cross-sectional view of an electronic apparatus 100 according to one or more embodiments of the present disclosure.

Referring to FIG. 2, the electronic apparatus 100 may include an organic photodetector 400 (e.g., in a light detection region) and a light-emitting device 500 (e.g., in an emission region) between a substrate 601 and a substrate 602.

The substrate 601 and the substrate 602 may each independently be a flexible substrate, a glass substrate, or a metal substrate. In some embodiments, a buffer layer and a thin-film transistor may be on the substrate 601.

The buffer layer serves to prevent infiltration of impurities through the

substrate 601 and provide a flat surface on the substrate 601. The thin-film transistor may be on the buffer layer, and may include an activation layer, a gate electrode, a source electrode, and a drain electrode.

The thin-film transistor may be electrically connected to the light-emitting device 500 to drive the light-emitting device. For example, a second pixel electrode 510 of the light-emitting device 500 may be electrically connected with the source electrode or the drain electrode of the thin-film transistor.

Another thin-film transistor may be electrically connected to the organic photodetector 400. For example, a first pixel electrode 410 of the organic photodetector 400 may be electrically connected with the source electrode or the drain electrode of the another thin-film transistor.

The organic photodetector 400 may include the first pixel electrode 410, a hole injection layer 420, a hole transport layer 432, an activation layer 440, an electron transport layer 450, and a counter electrode 470.

In one or more embodiments, the first pixel electrode 410 may be an anode, and the counter electrode 470 may be a cathode. That is, as the organic photodetector 400 is driven by applying a reverse bias across the first pixel electrode 410 and the counter electrode 470, the electronic apparatus 100 may detect light incident onto the organic photodetector 400, generate charges, and extract the charges as a current.

The light-emitting device 500 may include the second pixel electrode 510, the hole injection layer 420, the hole transport layer 432, an emission layer 540, the electron transport layer 450, and the counter electrode 470.

In one or more embodiments, the second pixel electrode 510 may be an

anode, and the counter electrode 470 may be a cathode. That is, in the light-emitting device 500, holes injected from the second pixel electrode 510 and electrons injected from the counter electrode 470 recombine in the emission layer 540 to generate excitons, which generate light by decaying from an excited state to a ground state. For descriptions of the first pixel electrode 410 and the second pixel

electrode 510, the descriptions on the first electrode 110 provided herein may be referred to.

A pixel define layer 405 may be formed at the edge portions of the first pixel electrode 410 and the second pixel electrode 510. The pixel define layer 405 defines a pixel region, and may electrically insulate the first pixel electrode 410 and the second pixel electrode 510. The pixel define layer 405 may include, for example, one or more suitable organic insulating materials (for example, silicone-based materials, and/or the like), inorganic insulating materials, or organic/inorganic composite insulating materials. The pixel define layer 405 may be a transmissive film that transmits visible light, or a blocking film that blocks visible light.

The hole injection layer 420 and the hole transport layer 432, which are common layers, may be formed sequentially on the first pixel electrode 410 and the second pixel electrode 510. The hole injection layer 420 and the hole transport layer 432 are respectively the same as described elsewhere in the present disclosure.

The activation layer 440 may be formed on the hole transport layer 432 to correspond to the light detection region. For descriptions of the activation layer 440, related descriptions provided herein may be referred to.

The emission layer 540 may be formed on the hole transport layer 432 to correspond to the emission region. In one or more embodiments, the light-emitting device 500 may further include, between the second pixel electrode 510 and the emission layer 540, an electron blocking layer arranged in correspondence with the emission region.

As common layers for the entirety of the light detection region and emission region, the electron transport layer 450 and the counter electrode 470 are sequentially formed on the activation layer 440 and the emission layer 540. For descriptions of the electron transport layer 450 and the counter electrode 470, the descriptions of the electron transport layer 130 and the second electrode 170 provided herein may be referred to.

The hole injection layer 420, the hole transport layer 432, and the electron transport layer 450 may each be located throughout the light detection region and the emission region.

As such, a manufacturing process of the electronic apparatus 100 may be

simplified by arranging common layers for the organic photodetector 400 and the light-emitting device 500, each of functional layer materials utilized in the light-emitting device 500 may also be utilized for the organic photodetector 400, and thus, the organic photodetector 400 may be provided in-pixel in the electronic apparatus.

In one or more embodiments, an electron injection layer may be further included between the electron transport layer 450 and the counter electrode 470.

In one or more embodiments, a capping layer may be located on the counter electrode 470. A material that may be utilized for the capping layer may include an organic material and/or an inorganic material as described above. The capping layer may facilitate efficient emission of light generated from the light-emitting device 500, in addition to having a protective function for the organic photodetector 400 and the light-emitting device 500.

An encapsulation portion 490 may be located on the capping layer. The encapsulation portion 490 may be located on the organic photodetector 400 and the light-emitting device 500 to protect the organic photodetector 400 and the light-emitting device 500 from mixture and/or oxygen. The encapsulation portion 490 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, and/or the like), an epoxy-based resin (for example, aliphatic glycidyl ether (AGE), and/or the like), or any combination thereof; or a combination of the inorganic film and the organic film.

In one or more embodiments, the electronic apparatus 100 may be, for example, a display apparatus. The electronic apparatus 100 includes both (e.g., simultaneously) the organic photodetector 400 and the light-emitting device 500, and thus, may be a display apparatus with a light detection function.

In FIG. 2, the electronic apparatus 100 is illustrated as including one light-emitting device 500, but, as shown in FIG. 3, an electronic apparatus 100a according to one or more embodiments may include an organic photodetector 400, a first light-emitting device 501, a second light-emitting device 502, and a third light-emitting device 503.

Components illustrated in FIG. 3 may be understood by referring to the descriptions of the electronic apparatus 100.

The first light-emitting device 501 may include a second pixel electrode 511,

a hole injection layer 420, a hole transport layer 432, a first emission layer 541, an electron transport layer 450, and a counter electrode 470.

The second light-emitting device 502 may include a third pixel electrode 512, the hole injection layer 420, the hole transport layer 432, a second emission layer 542, the electron transport layer 450, and the counter electrode 470.

The third light-emitting device 503 may include a fourth pixel electrode 513, the hole injection layer 420, the hole transport layer 432, a third emission layer 543, the electron transport layer 450, and the counter electrode 470.

The second pixel electrode 511, the third pixel electrode 512, and the fourth pixel electrode 513 are arranged to correspond to a first light emission region, a second light emission region, and a third light emission region, respectively, and will be understood with reference to the descriptions of the first electrode 110 provided herein.

The first emission layer 541 is arranged in correspondence with the first light emission region and is to emit a first color light, the second emission layer 542 is arranged in correspondence with the second light emission region and is to emit a second color light, and the third emission layer 543 is arranged in correspondence with the light emission region and is to emit a third color light.

A maximum emission wavelength of the first color light, a maximum emission wavelength of the second color light, and a maximum emission wavelength of the third color light may be identical to or different from each other. For example, in one or more embodiments, the maximum emission wavelength of the first color light and the maximum emission wavelength of the second color light may each be greater than the maximum emission wavelength of the third color light.

In one or more embodiments, the first color light may be red light, the second color light may be green light, and the third color light may be blue light, but embodiments of the present disclosure are not limited thereto. Accordingly, the electronic apparatus 100a is capable of full-color emission. The first color light, the second color light, and the third color light are not limited to red light, green light, and blue light, respectively, and may be any combination of light of different colors, as long as mixed light thereof is white light.

The organic photodetector 400, the first light-emitting device 501, the second light-emitting device 502, and the third light-emitting device 503 may be subpixels constituting a single pixel. In one or more embodiments, one pixel may include at least one organic photodetector 400.

In one or more embodiments, the electronic apparatus 100a may be a display apparatus. The electronic apparatus 100a may include all the organic photodetector 400 and the first light-emitting device 501, the second light-emitting device 502, and the third light-emitting device 503, and thus, may be a full-color display apparatus with a light detection function.

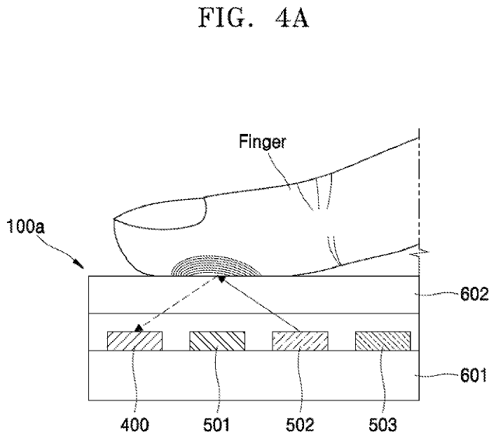

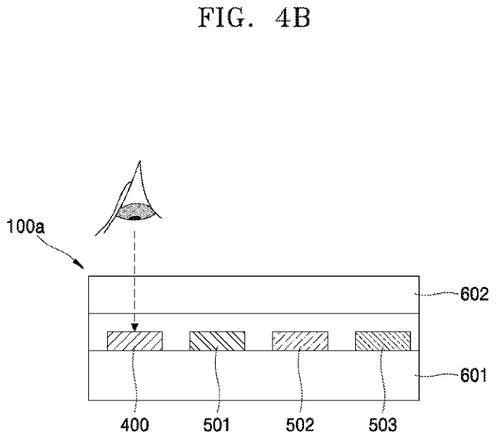

### Descriptions of FIG. 4A and FIG. 4B

In an electronic apparatus 100a shown in FIG. 4A, an organic photodetector 400 and light-emitting devices 501, 502, and 503 may be arranged between a substrate 601 and a substrate 602.

For example, in some embodiments, red light, green light, and blue light may be emitted from the light-emitting device 501, the light-emitting device 502, and the light-emitting device 503, respectively.

The electronic apparatus 100a according to one or more embodiments may have a function to detect, for example, a fingerprint of a finger, which is an object in contact with the electronic apparatus. In some embodiments, as shown in FIG. 4A, at least some light emitted from the light-emitting device 502 and reflected by a fingerprint of a user may be re-incident on the organic photodetector 400, and thus, the organic photodetector 400 may detect the reflected light. Ridges in the fingerprint pattern of a finger are in close contact with the substrate 602, and thus, the organic photodetector 400 may acquire the fingerprint pattern of the user. Although FIG. 4A shows an embodiment in which information of an object being in contact with the electronic apparatus 100a is obtained by utilizing light emitted from the second light-emitting device 502, light emitted from the first light-emitting device 501 and/or light emitted from the third light-emitting device 503 may also be utilized in the same manner when obtaining information by utilizing emitted light.

In one or more embodiments, as shown in FIG. 4B, the electronic apparatus 100a according to one or more embodiments may detect an object that is not in contact with the electronic apparatus 100a.

### Manufacturing Method

The layers included in the hole transport region, the activation layer, and the layers included in the electron transport region may each be formed in certain regions by utilizing one or more suitable methods selected from vacuum deposition, spin coating, casting, Langmuir-Blodgett (LB) deposition, ink-jet printing, laser-printing, and laser-induced thermal imaging (LITI).

When the layers of the hole transport region, the activation layer, and the layers of the electron transport region are each formed by vacuum deposition, deposition conditions may be selected from within a deposition temperature of about 100° C. to about 500° C., a vacuum degree of about 10⁻⁸ torr to about 10⁻³ torr, and a deposition rate of about 0.01 Å/sec to about 100 Å/sec, in consideration of a material and structure of a layer to be formed.

### Definition of Terms

The term “C₃-C₆₀ carbocyclic group” as utilized herein refers to a cyclic group including (e.g., consisting of) carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group including (e.g., consisting of) one (e.g., only one) ring or a polycyclic group in which two or more rings are condensed with each other. For example, the C₁-C₆₀ heterocyclic group has 3 to 61 ring-forming atoms.

The term “cyclic group” as utilized herein may include both the C₃-C₆₀ carbocyclic group and the C₁-C₆₀ heterocyclic group.

The term “π electron-rich C₃-C₆₀ cyclic group” as utilized herein refers to a

cyclic group that has three to sixty carbon atoms and does not include *—N═*′ as a ring-forming moiety, and the term “π electron-deficient nitrogen-containing C₁-C₆₀ cyclic group” as utilized herein refers to a heterocyclic group that has one to sixty carbon atoms and includes *—N′*′ as a ring-forming moiety.

For example, the C₃-C₆₀ carbocyclic group may be i) Group T1 (one or more of the selected groups in Group T1) or ii) a condensed cyclic group in which two or more of Group T1 (two or more the selected groups in Group T1) are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) Group T2, ii) a condensed cyclic group in which two or more of Group T2 are condensed with each other, or iii) a condensed cyclic group in which at least one Group T2 (at least one of the selected group in Group T2) and at least one Group T1 (at least one of the selected group in Group T1) are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.), the π electron-rich C₃-C₆₀ cyclic group may be i) Group T1, ii) a condensed

cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1 ]pentane group, a bicyclo[2.1.1]hexane group, a bicyclo[2.2.2]octane group, or a benzene group,

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

Group T3 may be a furan group, a thiophene group, a 1H-pyrrole group, a silole group, or a borole group, and

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as utilized herein may refer to a group condensed to any cyclic group, a monovalent group, or a polyvalent group (for example, a divalent group, a trivalent group, a tetravalent group, etc.) according to the structure of a formula for which the corresponding term is utilized. In one or more embodiments, “a benzene group” may be a benzo group, a phenyl group, a phenylene group, or the like, which may be easily understand by one of ordinary skill in the art according to the structure of a formula including the “benzene group.”

Depending on context, in the present disclosure, a divalent group may refer or be a polyvalent group (e.g., trivalent, tetravalent, etc., and not just divalent) per, e.g., the structure of a formula in connection with which of the terms are utilized.

Non-limiting examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a Cs-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₃-C₆₀ carbocyclic group and the divalent C₁-C₆₀ heterocyclic group may include a Cs-C₁₀ cycloalkylene group, a

C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as utilized herein refers to a linear or branched aliphatic hydrocarbon monovalent group that has one to sixty carbon atoms, and non-limiting examples thereof may be a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having substantially the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of a C₂-C₆₀ alkyl group, and non-limiting examples thereof may be an ethenyl group, a propenyl group, and a butenyl group. The term “C₂-C₆₀ alkenylene group” as utilized herein refers to a divalent group having substantially the same structure as the C₂-C₆₀ alkenyl group.

The term “C₂-C₆₀ alkynyl group” as utilized herein refers to a monovalent

hydrocarbon group having at least one carbon-carbon triple bond in the middle or at the terminus of a C₂-C₆₀ alkyl group, and non-limiting examples thereof may include an ethynyl group, a propynyl group, and/or the like. The term “C₂-C₆₀ alkynylene group” as utilized herein refers to a divalent group having substantially the same structure as the C₂-C₆₀ alkynyl group.

The term “C₁-C₆₀ alkoxy group” as utilized herein refers to a monovalent group represented by-OA₁₀₁ (wherein A₁₀₁ is a C₁-C₆₀ alkyl group), and non-limiting examples thereof may include a methoxy group, an ethoxy group, and an isopropyloxy group.

The term “C₃-C₁₀ cycloalkyl group” as utilized herein refers to a monovalent saturated hydrocarbon monocyclic group including 3 to 10 carbon atoms. Non-limiting examples of the C₃-C₁₀ cycloalkyl group as utilized herein may include a cyclopropyl group, a cyclobutyl group, a cyclopentyl group, a cyclohexyl group, a cycloheptyl group, a cyclooctyl group, an adamantyl group, a norbornyl (bicyclo[2.2.1]heptyl) group, a bicyclo[1.1.1]pentyl group, a bicyclo[2.1.1]hexyl group, or a bicyclo[2.2.2]octyl group. The term “C₃-C₁₀ cycloalkylene group” as utilized herein refers to a divalent group having substantially the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and non-limiting examples thereof may be a 1,2,3,4-oxatriazolidinyl group, a tetrahydrofuranyl group, and a tetrahydrothiophenyl group. The term “C₁-C₁₀ heterocycloalkylene group” as utilized herein refers to a divalent group having substantially the same structure as the C₁-C₁₀ heterocycloalkyl group.

The term “C₃-C₁₀ cycloalkenyl group” utilized herein refers to a monovalent cyclic group that has three to ten carbon atoms and at least one carbon-carbon double bond in the ring thereof and no aromaticity, and non-limiting examples thereof may be a cyclopentenyl group, a cyclohexenyl group, and a cycloheptenyl group. The term “C₃-C₁₀ cycloalkenylene group” as utilized herein refers to a divalent group having substantially the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one double bond in the cyclic structure thereof. Non-limiting examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having substantially the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Non-limiting examples of the C₆-C₆₀ aryl group may be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Non-limiting examples of the C₁-C₆₀ heteroaryl group may be a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group having two or more rings condensed and only carbon atoms (for example, having 8 to 60 carbon atoms) as ring-forming atoms, wherein the molecular structure (e.g., the group structure) as a whole is non-aromatic. Non-limiting examples of the monovalent non-aromatic condensed polycyclic group may be an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having substantially the same structure as the monovalent non-aromatic condensed polycyclic group described above.

The term “monovalent non-aromatic condensed heteropolycyclic group” as utilized herein refers to a monovalent group having two or more rings condensed and carbon atoms (for example, having 1 to 60 carbon atoms) and at least one heteroatom as ring-forming atoms, wherein the molecular structure (e.g., the group structure) as a whole is non-aromatic. Examples of a monovalent non-aromatic condensed heteropolycyclic group include a 9,9-dihydroacridinyl group and a 9H-xanthenyl group. The term “divalent non-aromatic condensed heteropolycyclic group” as utilized herein refers to a divalent group having substantially the same structure as the monovalent non-aromatic condensed heteropolycyclic group described above.

The term “C₆-C₆₀ aryloxy group” as utilized herein indicates -OA₁₀₂ (wherein A₁₀₂ is a C₆-C₆₀ aryl group), and the term “C₆-C₆₀ arylthio group” as utilized herein indicates -SA₁₀₃ (wherein A₁₀₃ is a C₆-C₆₀ aryl group).

The term “C₇-C₆₀ arylalkyl group” utilized herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroarylalkyl group” utilized herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as utilized herein refers to:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ as utilized herein may each independently be: hydrogen; deuterium —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Examples of the heteroatom may be O, S, N, P, Si, B, Ge, Se, and any combinations thereof.

The term “third-row transition metal” as utilized herein includes hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “But” as utilized herein refers to a tert-butyl group, and the term “OMe” as utilized herein refers to a methoxy group.

The term “biphenyl group” as utilized herein refers to “a phenyl group substituted with a phenyl group.” In some embodiments, the “biphenyl group” is a substituted phenyl group having a C₆-C₆₀ aryl group as a substituent.

The term “terphenyl group” as utilized herein refers to “a phenyl group substituted with a biphenyl group”. In some embodiments, the “terphenyl group” is a substituted phenyl group having, as a substituent, a C₆-C₆₀ aryl group substituted with a C₆-C₆₀ aryl group.

### EXAMPLES

### Synthesis Example 1 Compound 13

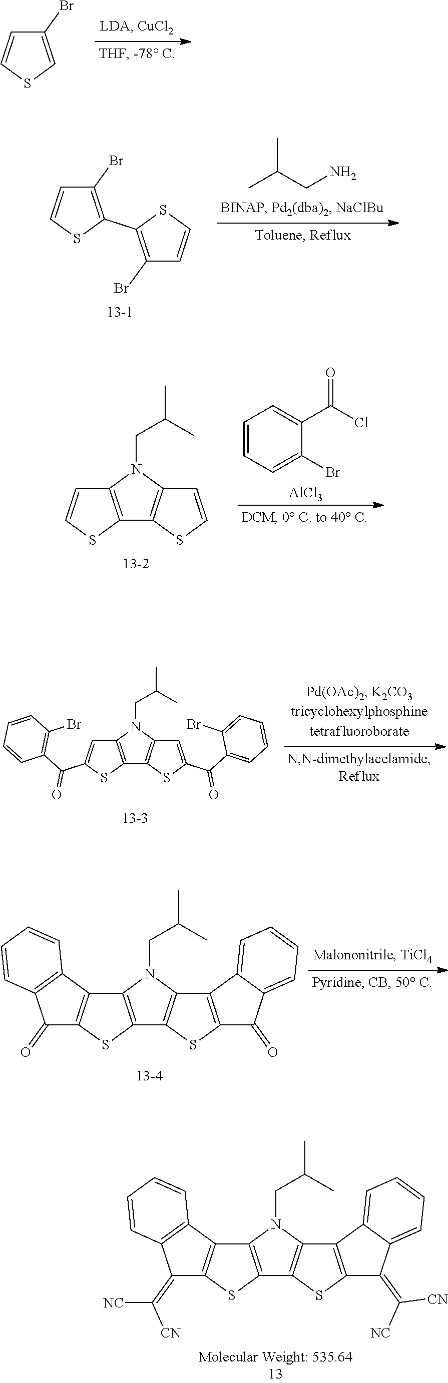

### Synthesis of Intermediate 13-1

3-bromothiophene (30 g, 181.01 mmol) was dissolved in anhydrous tetrahydrofuran (THF) (150 mL) in a round flask and then stirred at-78° C. for 15 minutes. Lithium diisopropylamide (LDA) (184.01 mmol) was added dropwise thereto at the same temperature. After adding LDA, the mixture was stirred for 1 hour, and CuCl₂ was added thereto, followed by stirring for 1 hour. After the reaction was continued at room temperature for 6 hours, quenching was performed by utilizing an ammonium chloride solution, and materials dissolved in an organic solvent were extracted by utilizing dichloromethane (DCM) and H₂O. Remaining moisture was removed therefrom by adding MgSO₄, and filtration was performed. A filtered solution was dissolved in DCM, and a solvent was removed by utilizing a rotary evaporator. A crude product was separated through column chromatography by utilizing hexane as an eluent to obtain Intermediate 13-1 (yield: 19%).

### Synthesis of Intermediate 13-2

Intermediate 13-1 (100 mg, 1 eq), sodium tert-butoxide (NaOtBu) (3.0 eq), tris(dibenzylideneacetone)dipalladium(0) (Pd₂(dba)₃) (0.12 eq), and 2,2′-bis(diphenylphosphino)-1,1′-binaphthyl (BINAP) (0.48 eq) were added to a microwave vial, and then dissolved in anhydrous toluene (5 mL) in a nitrogen condition, followed by stirring at 35° C. for 1 hour. Isobutyl amine (1.3 eq) was added thereto, and reaction was performed for 12 hours. The temperature was raised to 110° C., and the mixture was stirred for 12 hours. After extraction was performed thereon by utilizing diethyl ether and H₂O, remaining moisture was removed therefrom by utilizing MgSO₄, and filtration was conducted. A solvent of the filtered solution was removed by utilizing a rotary evaporator. A crude product was separated through column chromatography by utilizing hexane and DCM (a volume ratio of 10:1) as an eluent to obtain Intermediate 13-2 (yield: 73%).

### Synthesis of Intermediate 13-3

Intermediate 13-2 (100 mg, 0.425 mmol) was dissolved in anhydrous DCM (1 mL) to obtain Solution 1. In a nitrogen condition, AlCl₃ (3 eq) and 2-bromobenzoyl chloride (3 eq) were added to anhydrous DCM (3 mL) and then stirred at 0° C. for 1 hour to obtain Solution 2. Solution 1 was added dropwise to Solution 2 at 0° C. and then stirred for 30 minutes. After the reaction was continued at room temperature for 12 hours, materials dissolved in an organic solvent were extracted by utilizing DCM and H₂O. Remaining moisture was removed therefrom by adding MgSO₄, and filtration was performed. A filtered solution was dissolved in DCM, and a solvent was removed by utilizing a rotary evaporator. A crude product was separated through column chromatography by utilizing chloroform as an eluent to obtain Intermediate 13-3 (yield: 50%).

### Synthesis of Intermediate 13-4

In a nitrogen condition, Intermediate 13-3 (100 mg, 0.166 mmol), palladium(II)acetate(0.2 eq), tricyclohexylphosphonium tetrafluoroborate (0.4 eq), and K₂CO₃ (5.0 eq) were dissolved in N,N-dimethylacetamide (DMA, 8 mL) and then stirred at 130° C. for 12 hours. After extraction was performed thereon by utilizing ethyl acetate, the filtrate was washed three times by utilizing brine to remove remaining DMA. Remaining moisture was removed therefrom by adding MgSO₄, and filtration was performed. A filtered solution was dissolved in DCM, and a solvent was removed by utilizing a rotary evaporator. A crude product was separated through column chromatography by utilizing DCM as an eluent and then washed several times by utilizing hexane and methanol to obtain Intermediate 13-4 (yield: 43%).

### Synthesis of Compound 13

After Intermediate 13-4 and malononitrile (5 eq) were dissolved in anhydrous chlorobenzene (6 mL), pyridine (25 eq) and TiCl₄ (1.25 eq) were added dropwise thereto. The mixture was stirred at 50° C. for 5 hours, and extraction was performed thereon by utilizing water and DCM. After water in the extracted solution was removed by utilizing anhydrous Na₂SO₄, filtration was performed thereon, and a solvent in the solution was removed by utilizing a rotary evaporator. Then, Compound 13 was obtained through column chromatography.

### Synthesis Example 2 (Compound 37)

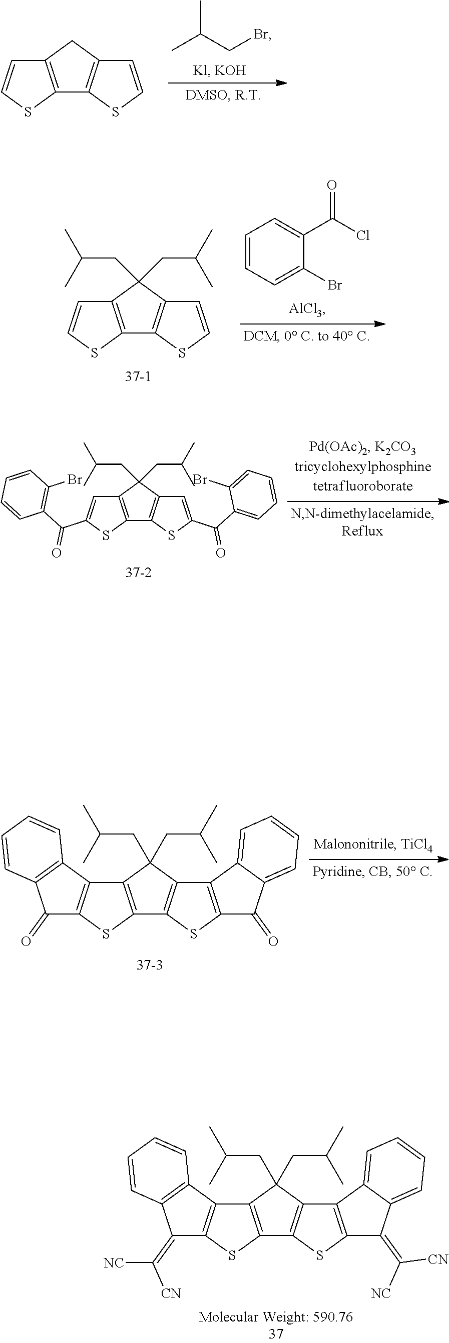

### Synthesis of Intermediate 37-1

After cyclopentadithiophene (200 mg) was dissolved in anhydrous dimethyl sulfoxide (DMSO) (20 mL) in a round flask, isobutyl bromide (2 eq) and KI (0.03 eq) were added thereto. The mixture was cooled to 0° C., and KOH was added thereto in a nitrogen condition. After stirring the mixture at room temperature for 12 hours, quenching was performed by adding H₂O to the reaction product, and materials dissolved in an organic solvent were extracted by utilizing ether, H₂O, and brine. Remaining moisture was removed by adding MgSO₄, and a solvent was removed by utilizing a rotary evaporator. Then, separation was conducted through column chromatography by utilizing hexane as an eluent to obtain Intermediate 37-1 (yield: 46%)

### Synthesis of Compound 37

Intermediate 37-2 was obtained in substantially the same manner as in Synthesis of Intermediate 13-3, except that Intermediate 37-1 was utilized instead of Intermediate 13-2 when synthesizing Intermediate 37-2.

Intermediate 37-3 was obtained in substantially the same manner as in Synthesis of Intermediate 13-4, except that Intermediate 37-2 was utilized instead of Intermediate 13-3 when synthesizing Intermediate 37-3.

Compound 37 was obtained in substantially the same manner as in Synthesis of Compound 13, except that Intermediate 37-3 was utilized instead of Intermediate 13-4 when synthesizing Compound 37.

Synthesis methods of compounds other than the compounds of Synthesis Examples 1 and 2 may be easily recognized by those skilled in the art by referring to the synthesis paths and source materials.

### Evaluation Example 1

The HOMO energy level, LUMO energy level, optical band gap, maximum absorption wavelength, and extinction coefficient of each of the compounds of Examples 1 and 2 and Comparative Examples 1 to 3 were evaluated by utilizing the methods described in Table 1, and the results thereof are shown in Table 2.

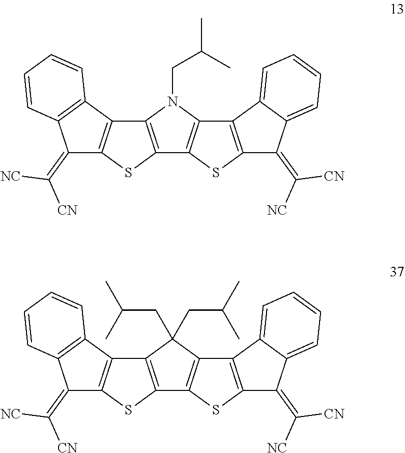 [SMILES: CC(C)Cn1c2c3c(sc2c2sc4c(c21)-c1ccccc1C4=C(C#N)C#N)C(=C(C#N)C#N)c1ccccc1-3 ; CC(C)CC1(CC(C)C)c2c(sc3c2-c2ccccc2C3=C(C#N)C#N)-c2sc3c(c21)-c1ccccc1C3=C(C#N)C#N]

### Comparative Example Compound 1

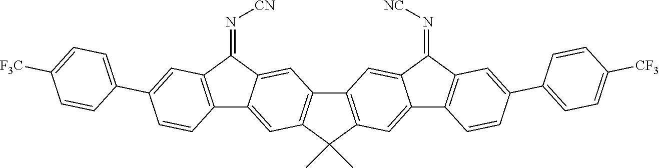 [SMILES: CC1(C)c2cc3c(cc2-c2cc4c(cc21)-c1ccc(-c2ccc(C(F)(F)F)cc2)cc1/C4=N/C#N)/C(=N\C#N)c1cc(-c2ccc(C(F)(F)F)cc2)ccc1-3]

### Comparative Example Compound 2

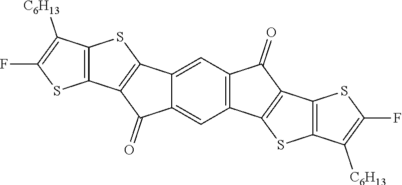

### Comparative Example Compound 3

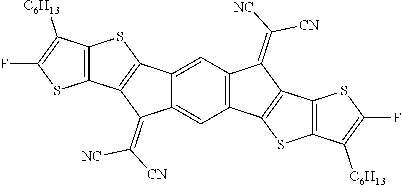

TABLE 1

| | |
|---|---|
| HOMO energy level | By utilizing cyclic voltammetry (CV) (electrolyte: 0.1M |
| evaluation method | Bu₄NPF₆/solvent: dimethyl formamide (DMF)/electrode: 3- |
| | electrode system (working electrode: glassy carbon (GC), |
| | reference electrode: Ag/AgCl, and auxiliary electrode: Pt)), |
| | the potential (V)-current (A) graph of each compound was |
| | obtained, and then, from an oxidation onset of the graph, |
| | the HOMO energy level of each compound was calculated. |
| LUMO energy level | A potential (V)-current (A) graph of each compound was |
| evaluation method | obtained by utilizing cyclic voltammetry (CV) (electrolyte: 0.1M |
| | Bu₄NPF₆/solvent: dimethyl formamide (DMF)/electrode: |
| | 3 electrode system (working electrode: GC, reference |
| | electrode: Ag/AgCl, auxiliary electrode: Pt)), and then, from |
| | a reduction onset of the graph, the LUMO energy level of |
| | each compound was calculated. |
| Maximum absorption | After synthesizing molecular structures/compounds, |
| wavelength (λₘₐₓ(film)) | spectrum data according to wavelength for absorption |
| | region was obtained through measurement or simulation, |
| | and a wavelength showing a maximum absorbance in an |
| | absorption spectrum was calculated. |
| Extinction coefficient | After coating or depositing a material, by utilizing an UV |
| (ε*10⁵ (cm⁻¹)@λₘₐₓ) | visible measurement device, an absorption spectrum |
| | according to wavelength was measured. Then, a ratio of |
| | intensity of transmitted light in incident light was taken as a |
| | natural log value and then converted into a common log |
| | value, which was divided by a thickness value of measured |
| | sample to calculate an extinction coefficient. |

TABLE 2

| | | | Optical | | | |
|---|---|---|---|---|---|---|
| | | | band | OSC | | |
| | HOMO | LUMO | gap | (Oscillator | λₘₐₓ(film) | |
| Compound | (eV) | (eV) | (eV) | Strength) | (nm) | ε*10⁵(cm⁻¹)@λₘₐₓ |
| Synthesis | −6.11 | −3.91 | 2.20 | 0.404 | 638.4 | 2.79 |
| Example 1 | | | | | | |
| Synthesis | −6.22 | −4.05 | 2.17 | 0.495 | 636.1 | 3.42 |
| Example 2 | | | | | | |
| Comparative | −6.29 | −3.48 | 2.81 | 0.081 | 554.1 | 0.56 |
| Example 1 | | | | | | |
| Comparative | −5.63 | −3.37 | 2.26 | 0.084 | 745.0 | 0.58 |
| Example 2 | | | | | | |
| Comparative | −5.93 | −4.27 | 1.66 | 0.071 | 511.7 | 0.49 |
| Example 3 | | | | | | |

From Table 2, it is understood that the compounds of Synthesis Examples 1 and 2 each have a higher extinction coefficient than that of the compounds of Comparative Examples 1 to 3 and show a maximum absorption wavelength suitable for infrared or red wavelength region.

### Example 1

An ITO glass substrate (anode) was cut to a size of 50 mm×50 mm×0.5 mm, ultrasonically cleaned with isopropyl alcohol and pure water each for 15 minutes, and then cleaned by irradiation of ultraviolet rays and exposure to ozone for 10 minutes. Then, the ITO glass substrate was mounted to a vacuum deposition apparatus. HAT-CN was vacuum-deposited on the anode to form a hole injection layer of a thickness of 100 Å, and HT3 was vacuum-deposited on the hole injection layer to form a hole transport layer of a thickness of 1,250 Å.

Then, m-MTDATA was vacuum-deposited on the hole transport layer to form

an auxiliary layer having a thickness of 200 Å, Compound 13 was deposited on the auxiliary layer in a thickness of 80 Å, and N14 was deposited thereon in a thickness of 340 Å, thereby forming an activation layer.

Next, BAlq was vacuum-deposited thereon to form a buffer layer having a thickness of 50 Å, and ET1 was vacuum-deposited on the buffer layer to form an electron transport layer having a thickness of 300 Å. LiF was vacuum-deposited on the electron transport layer to form an electron injection layer having a thickness of 10 Å, and then MgAg having a thickness of 100 Å was deposited on the electron injection layer to form a cathode, thereby completing manufacture of an clorganic photodetector.

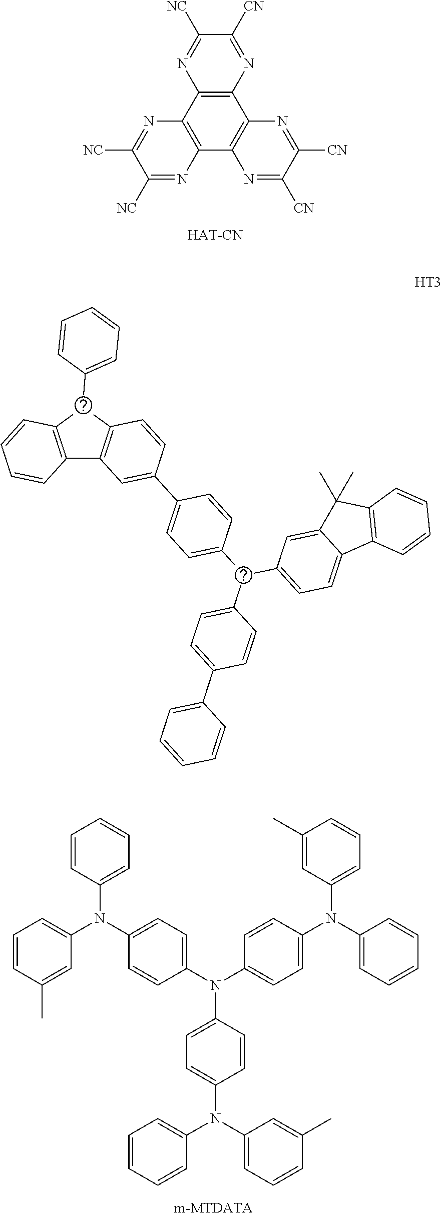 [SMILES: N#Cc1nc2c3nc(C#N)c(C#N)nc3c3nc(C#N)c(C#N)nc3c2nc1C#N ; CC1(C)c2ccccc2-c2ccc(C(c3ccc(-c4ccccc4)cc3)c3ccc(-c4ccc5c(c4)c4ccccc4n5-c4ccccc4)cc3)cc21 ; Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1]

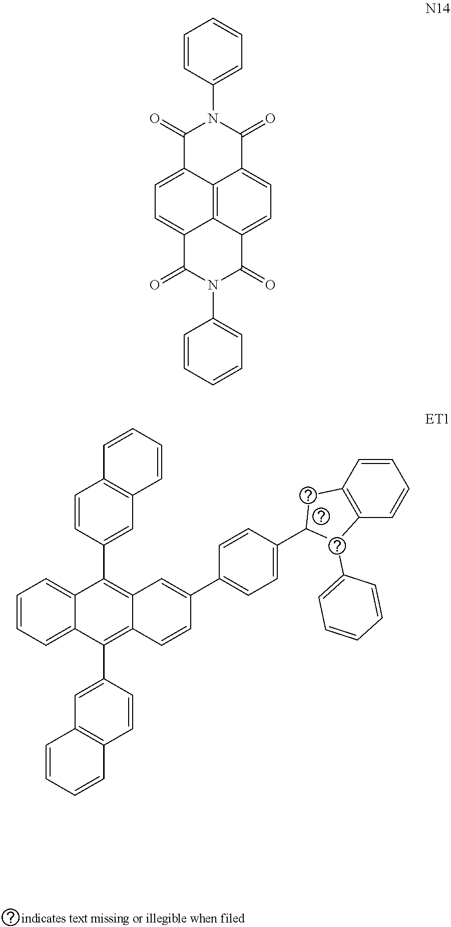

### Example 2 and Comparative Examples 1 to 3

Organic photodetectors of Example 2 and Comparative Examples 1 to 3 were each manufactured in substantially the same manner as in Example 1, except that Compound 37 and Comparative Compounds 1 to 3 were each utilized instead of Compound 13 when forming a respective activation layer.

### Evaluation Example 2

The external quantum efficiency (EQE) of each of the organic photodetectors manufactured in Examples 1 and 2 and Comparative Examples 1 to 3 was measured by utilizing an external quantum efficiency measurement device C9920-2-12 of Hamamatsu Photonics Inc., and the results thereof are shown in Table 3.

TABLE 3

| | EQE (%) |
|---|---|
| Example 1 | 32.9 |
| Example 2 | 40.3 |
| Comparative | 6.6 |
| Example 1 | |
| Comparative | 6.8 |
| Example 2 | |
| Comparative | 5.8 |
| Example 3 | |

From Table 3, it is understood that the organic photodetectors of Examples 1 and 2 each have better EQE than that of the organic photodetectors of Comparative Examples 1 to 3.

The condensed cyclic compound of the present disclosure may be an organic photoactive compound for vacuum deposition absorbing light of infrared or red region, and an organic photodetector and an electronic apparatus utilizing the condensed cyclic compound in an activation layer may have excellent or suitable efficiency.

In the present disclosure, although the terms “first,” “second,” “third,” etc., may be utilized herein to describe one or more elements, components, regions, and/or layers, these elements, components, regions, and/or layers should not be limited by these terms. These terms are only utilized to distinguish one component from another component.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5%of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The organic photodetector device, the light-emitting device, the display apparatus/device, the electronic apparatus, the electronic device, the electronic equipment, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that various changes in form and details may be made therein without departing from the spirit and scope as defined by the appended claims and equivalents thereof.

1 what is claimed is:

1. A condensed cyclic compound represented by Formula 1: 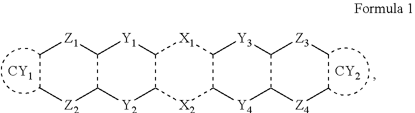
wherein, in Formula 1,
X₁ is a single bond, C(R₁), C(R₁)(R₂), Si(R₁)(R₂), Ge(R₁)(R₂), N(R₁), B(R₁), O, S, Se, or Te,
X₂ is a single bond, C(R₃), C(R₃)(R₄), Si(R₃)(R₄), Ge(R₃)(R₄), N(R₃), B(R₃), O, S, Se, or Te,
Y₁ to Y₄ are each independently a single bond, O, S, Se, or Te,
Z₁ is a single bond or 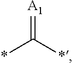
Z₂ is a single bond or 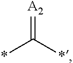
Z₃ is a single bond or 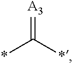
Z₄ is a single bond or 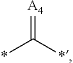
A₁ to A₄ are each independently O, S, C(R₅)(R₆), or N(R₅),
at least one selected from among X₁ and X₂ is not a single bond,
any one selected from among Y₁ and Y₂ is a single bond, and the other is not a single bond,
any one selected from among Y₃ and Y₄ is a single bond, and the other is not a single bond,
at least one selected from among Z₁ and Z₂ is not a single bond,
at least one selected from among Z₃ and Z₄ is not a single bond,
CY₁ and CY₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
R₁ to R₆ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a Cs-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C60 alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each independently indicate a binding site to a neighboring carbon atom.

2. The condensed cyclic compound of claim 1, wherein the condensed cyclic compound has a symmetric structure.

3. The condensed cyclic compound of claim 1, wherein the condensed cyclic compound has a molecular weight of 1,000 g/mol or less.

4. The condensed cyclic compound of claim 1, wherein the condensed cyclic compound has an optical band gap of 2.5 eV or less.

5. The condensed cyclic compound of claim 1, wherein X₁ is a single bond, C(R₁), C(R₁)(R₂), or N(R₁),
X₂ is a single bond, C(R₃), C(R₃)(R₄), or N(R₃),
at least one selected from among X₁ and X₂ is not a single bond, and
R₁ to R₄ are each independently the same as defined in Formula 1.

6. The condensed cyclic compound of claim 1, wherein Y₁ to Y₄ are each independently a single bond or S,
any one selected from among Y₁ and Y₂ is a single bond, and the other is not a single bond, and
any one selected from among Ys and Y₄ is a single bond, and the other is not a single bond.

7. The condensed cyclic compound of claim 1, wherein Z₁ to Z₄ are each independently a single bond,

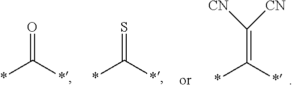

8. The condensed cyclic compound of claim 1, wherein any one selected from among Z₁ and Z₂ is a single bond, and the other is not a single bond, and
any one selected from among Z₃ and Z₄ is a single bond, and the other is not a single bond.

9. The condensed cyclic compound of claim 1, wherein CY₁ and CY₂ are each independently a cyclopentadiene group, a benzene group, a pentalene group, a naphthalene group, a phenalene group, a phenanthrene group, an anthracene group, a triphenylene group, a pyrene group, a chrysene group, a perylene group, a pentaphene group, a naphthacene group, an indene group, a fluorene group, a benzofluorene group, an indenophenanthrene group, an indenoanthracene group, a pyrrole group, a thiophene group, a thienothiophene group, a furan group, an indole group, a benzoindole group, a naphthoindole group, an iso-indole group, a benzoisoindole group, a naphthoisoindole group, a benzothiophene group, a benzofuran group, a carbazole group, a dibenzothiophene group, a dibenzofuran group, a pyrazole group, an imidazole group, a triazole group, a thiazole group, an isothiazole group, a benzopyrazole group, a benzimidazole group, a benzoxazole group, a benzothiazole group, a benzoisothiazole group, an indazole group, a pyridine group, a pyrimidine group, a pyrazine group, a pyridazine group, a triazine group, a quinoline group, an isoquinoline group, a benzoquinoline group, or a benzoisoquinoline group, each unsubstituted or substituted with at least one R₁₀ₐ.

10. The condensed cyclic compound of claim 1, wherein R₁ to R₆ are each independently:
hydrogen, deuterium, —F, —Cl, or a cyano group;
a C₁-C₆₀ alkyl group unsubstituted or substituted with at least one R₁₀ₐ or a C₁-C₆₀ alkoxy group unsubstituted or substituted with at least one R₁₀ₐ;
a C₃-C₆₀ carbocyclic group unsubstituted or substituted with at least one R₁₀ₐ or a C₁-C₆₀ heterocyclic group unsubstituted or substituted with at least one R₁₀ₐ; or
—C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

11. The condensed cyclic compound of claim 1, wherein the condensed cyclic compound is represented by any one selected from among Formulae 1-1 to 1-7: 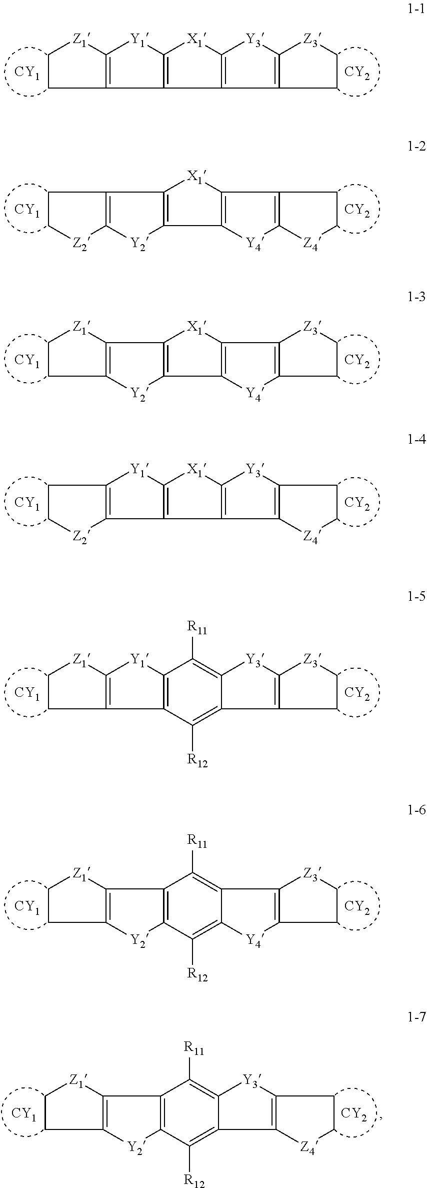
and wherein, in Formulae 1-1 to 1-7,
CY₁ and CY₂ are each the same as described in Formula 1,
X₁′ is C(R₁), C(R₁)(R₂), Si(R₁)(R₂), Ge(R₁)(R₂), N(R₁), B(R₁), O, S, Se, or Te,
Y₁′ to Y₄′ are each independently O, S, Se, or Te,
Z₁′is 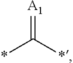
Z₂′ is 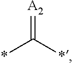
Z₃′ is 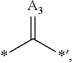
Z₄′ is 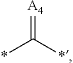
A₁ to A₄ are each independently O, S, C(R₅)(R₆), or N(R₅), and
R₁₁ and R₁₂ are each the same as described with respect to R₁ in Formula 1.

12. The condensed cyclic compound of claim 1, wherein CY₁ and CY₂ are each independently represented by any one selected from among Formulae 2-1 to 2-7: 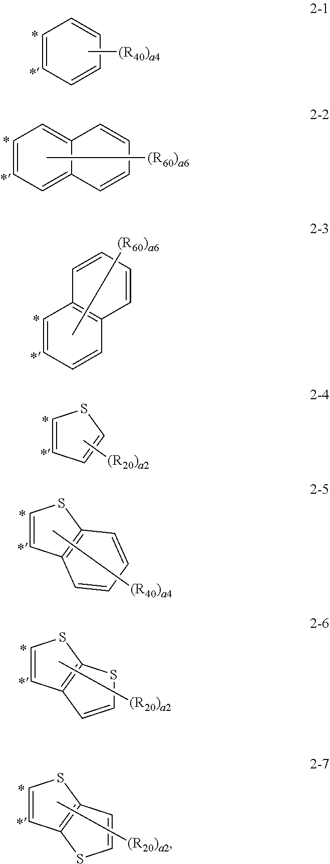
and wherein, in Formulae 2-1 to 2-7,
R₂₀, R₄₀, and R₆₀ are each the same as described with respect to R1 in Formula 1,
a2 is 1 or 2,
a4 is an integer from 1 to 4,
a6 is an integer from 1 to 6, and
* and ** are each independently carbon condensed with a neighboring ring.

13. The condensed cyclic compound of claim 1, wherein the condensed cyclic compound represented by Formula 1 is any one selected from among Compounds 1 to 72:

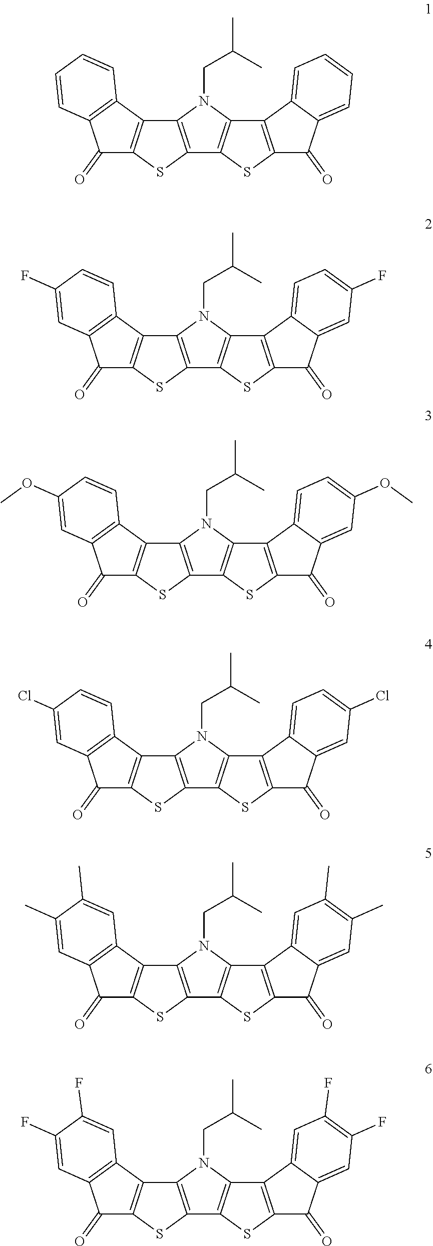

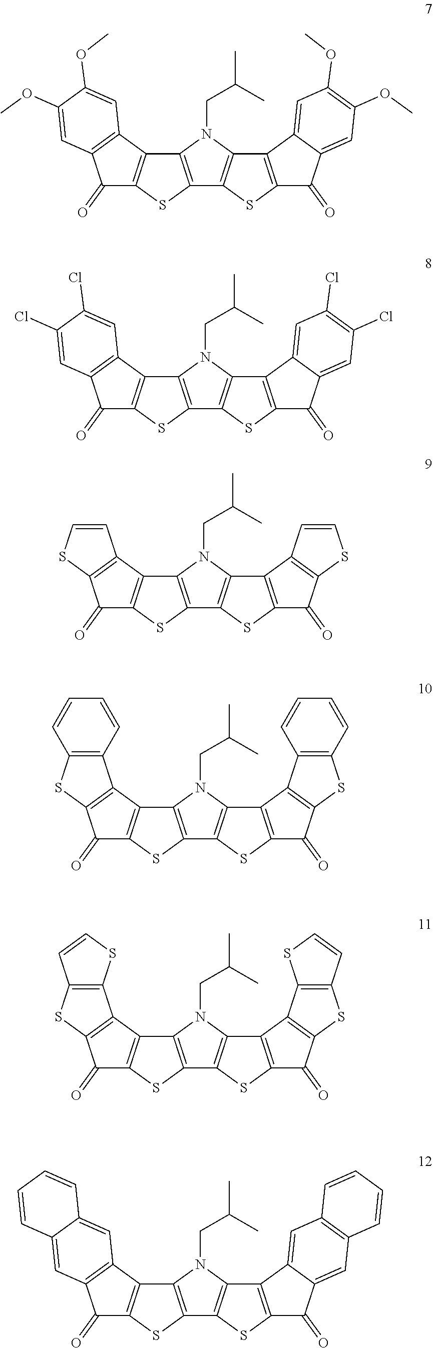

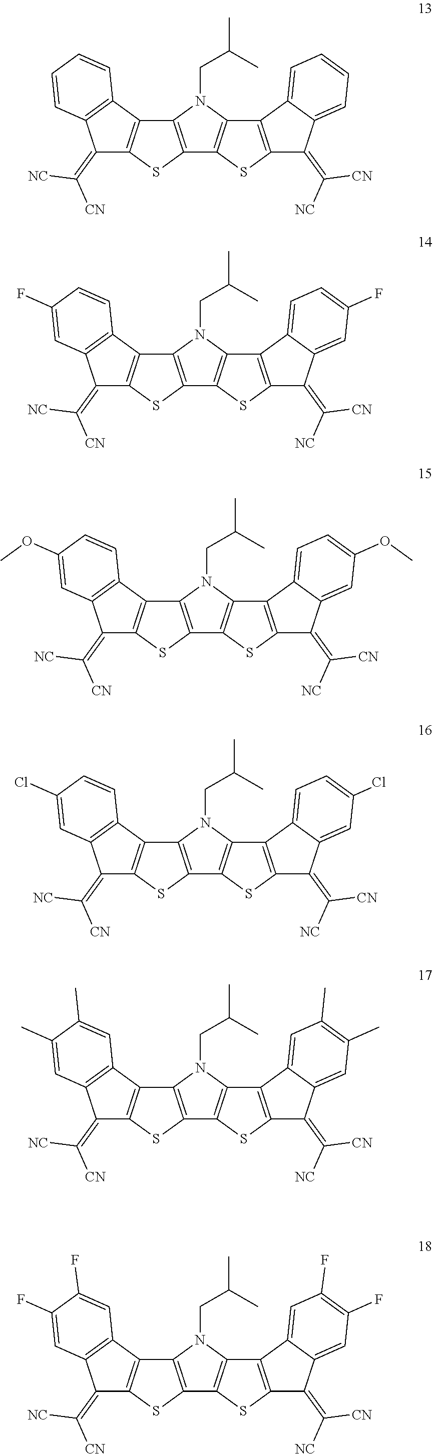

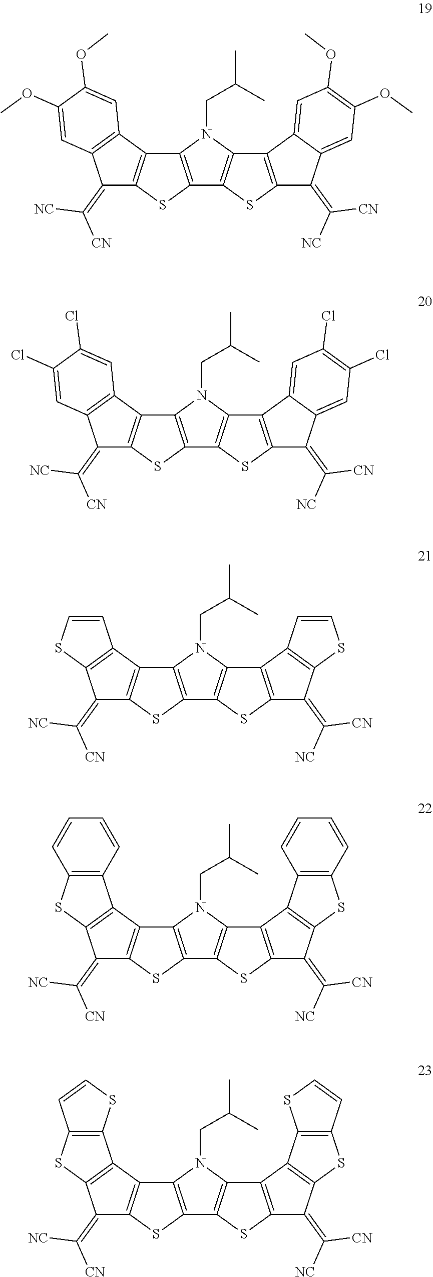

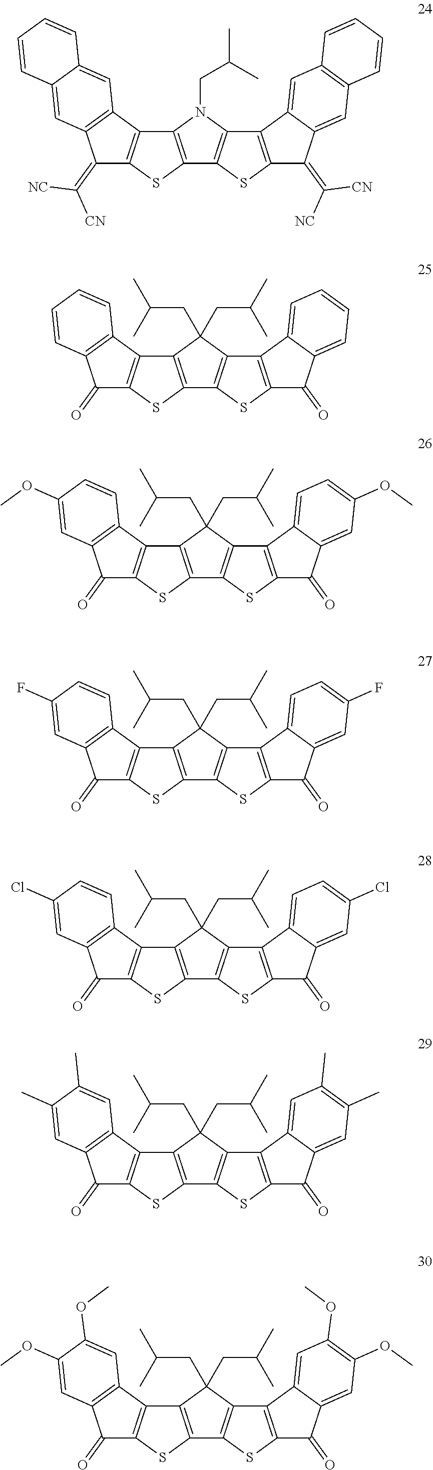

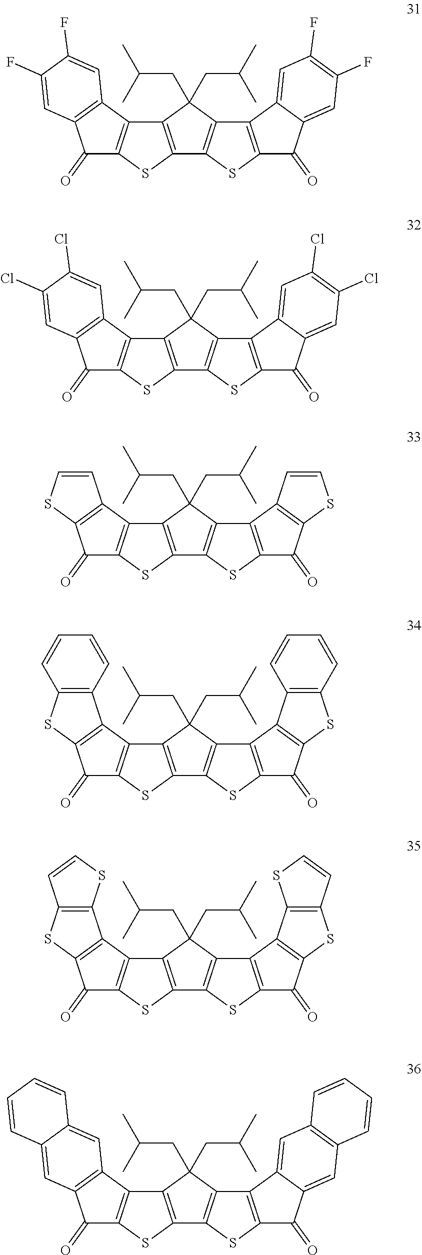 [SMILES: CC(C)CC1(CC(C)C)c2c(sc3c2-c2cc(F)c(F)cc2C3=O)-c2sc3c(c21)-c1cc(F)c(F)cc1C3=O ; CC(C)CC1(CC(C)C)c2c(sc3c2-c2cc(Cl)c(Cl)cc2C3=O)-c2sc3c(c21)-c1cc(Cl)c(Cl)cc1C3=O ; CC(C)CC1(CC(C)C)c2c(sc3c2-c2ccsc2C3=O)-c2sc3c(c21)-c1ccsc1C3=O ; CC(C)CC1(CC(C)C)c2c(sc3c2-c2c(sc4ccccc24)C3=O)-c2sc3c(c21)-c1c(sc2ccccc12)C3=O ; CC(C)CC1(CC(C)C)c2c(sc3c2-c2c(sc4ccsc24)C3=O)-c2sc3c(c21)-c1c(sc2ccsc12)C3=O ; CC(C)CC1(CC(C)C)c2c(sc3c2-c2cc4ccccc4cc2C3=O)-c2sc3c(c21)-c1cc2ccccc2cc1C3=O]

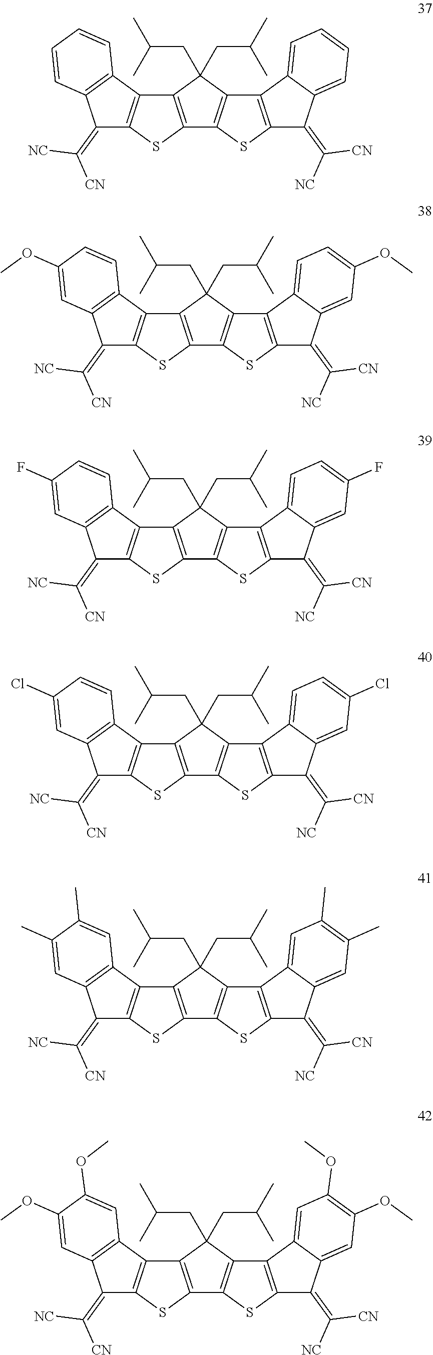

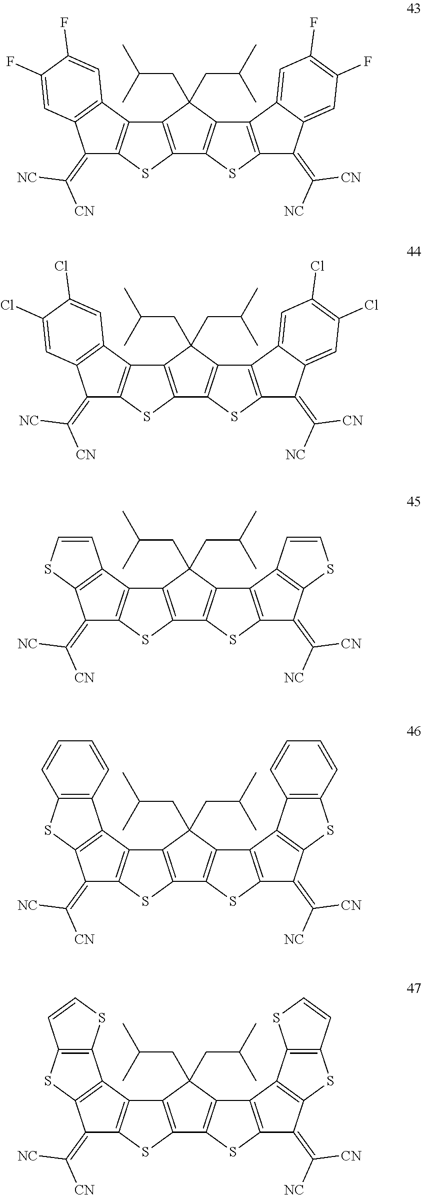 [SMILES: CC(C)CC1(CC(C)C)c2c(sc3c2-c2cc(F)c(F)cc2C3=C(C#N)C#N)-c2sc3c(c21)-c1cc(F)c(F)cc1C3=C(C#N)C#N ; CC(C)CC1(CC(C)C)c2c(sc3c2-c2cc(Cl)c(Cl)cc2C3=C(C#N)C#N)-c2sc3c(c21)-c1cc(Cl)c(Cl)cc1C3=C(C#N)C#N ; CC(C)CC1(CC(C)C)c2c(sc3c2-c2ccsc2C3=C(C#N)C#N)-c2sc3c(c21)-c1ccsc1C3=C(C#N)C#N ; CC(C)CC1(CC(C)C)c2c(sc3c2-c2c(sc4ccccc24)C3=C(C#N)C#N)-c2sc3c(c21)-c1c(sc2ccccc12)C3=C(C#N)C#N ; CC(C)CC1(CC(C)C)c2c(sc3c2-c2c(sc4ccsc24)C3=C(C#N)C#N)-c2sc3c(c21)-c1c(sc2ccsc12)C3=C(C#N)C#N]

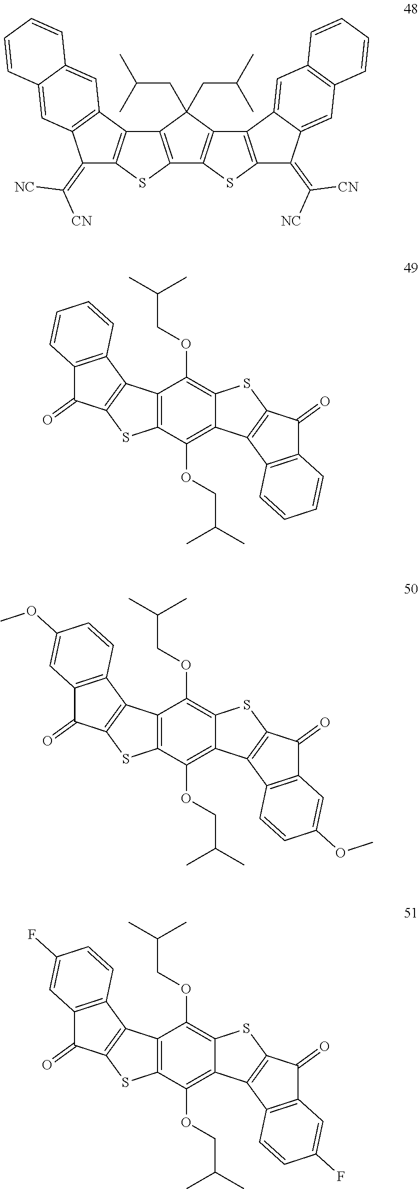

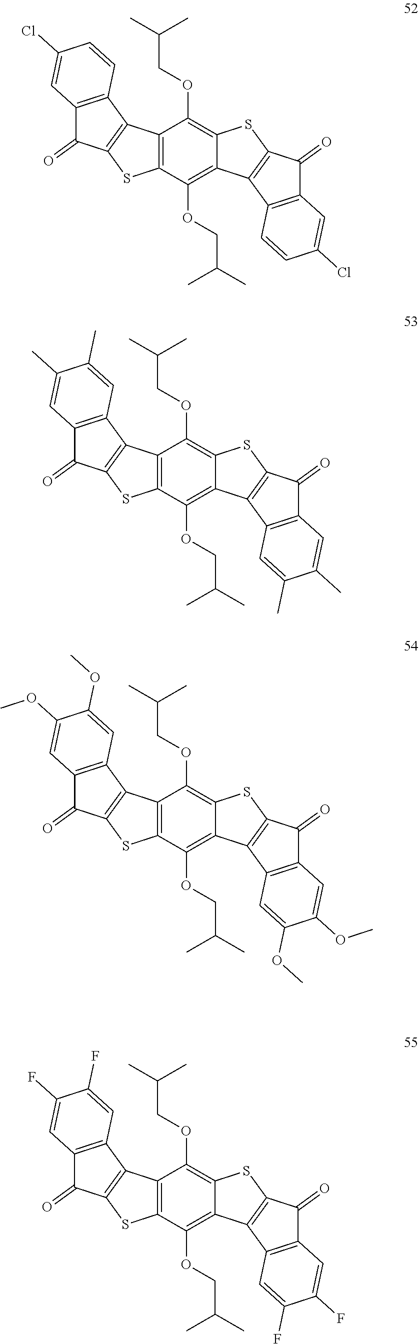

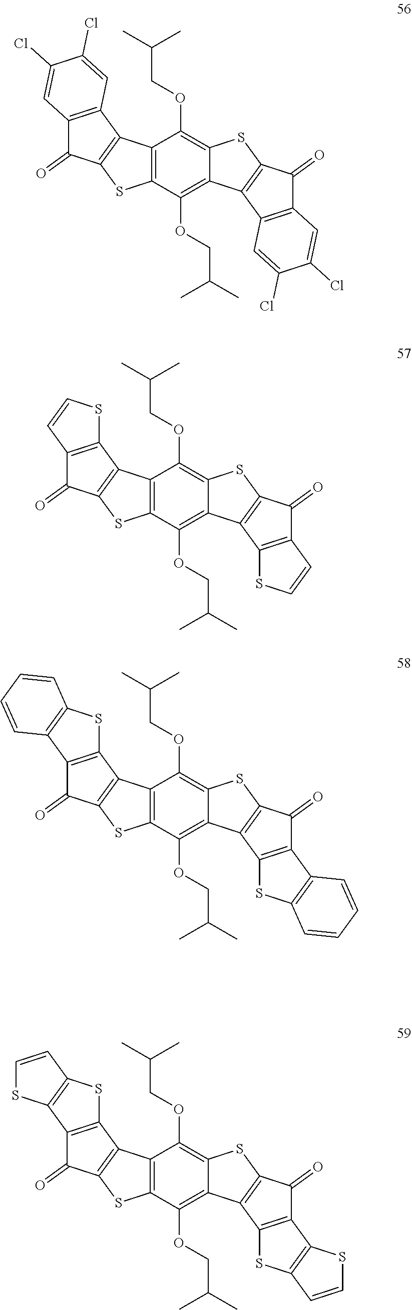

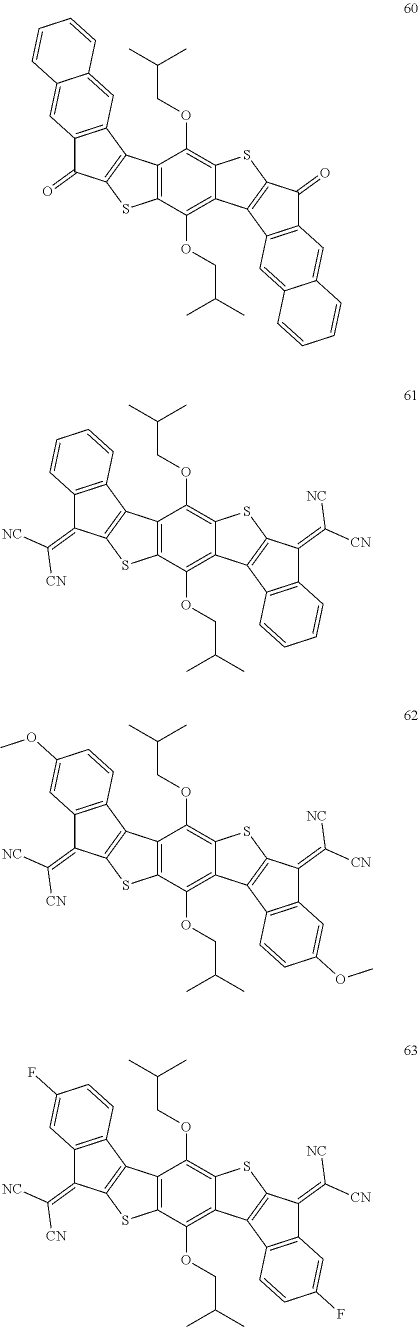

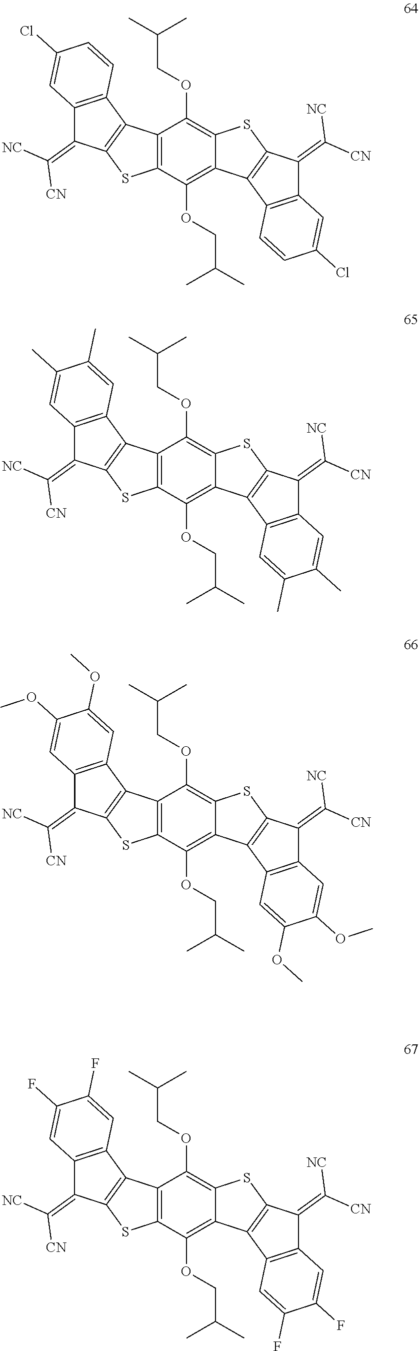

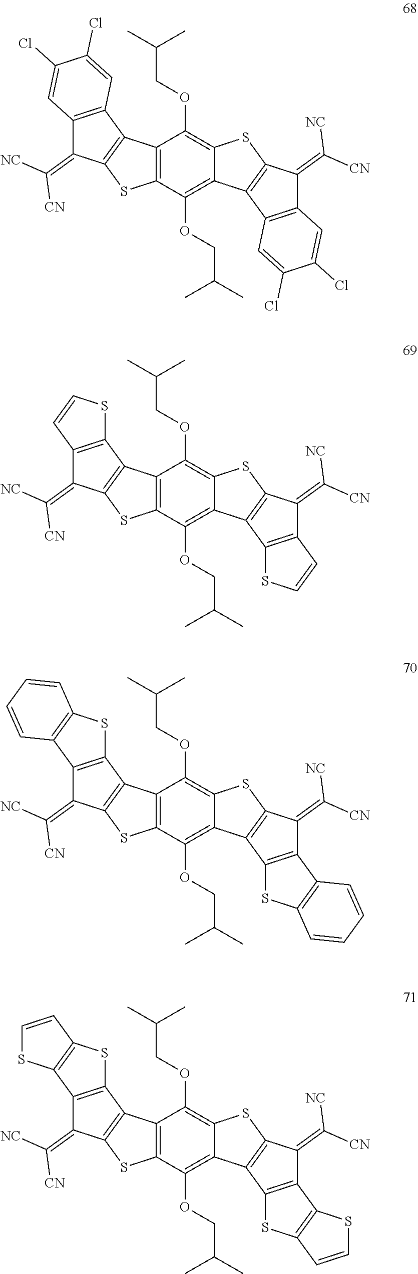 [SMILES: CC(C)COc1c2sc3c(c2c(OCC(C)C)c2sc4c(c12)-c1cc(Cl)c(Cl)cc1C4=C(C#N)C#N)-c1cc(Cl)c(Cl)cc1C3=C(C#N)C#N ; CC(C)COc1c2sc3c(c2c(OCC(C)C)c2sc4c(c12)-c1sccc1C4=C(C#N)C#N)-c1sccc1C3=C(C#N)C#N ; CC(C)COc1c2sc3c(c2c(OCC(C)C)c2sc4c(c12)-c1sc2ccccc2c1C4=C(C#N)C#N)-c1sc2ccccc2c1C3=C(C#N)C#N ; CC(C)COc1c2sc3c(c2c(OCC(C)C)c2sc4c(c12)-c1sc2ccsc2c1C4=C(C#N)C#N)-c1sc2ccsc2c1C3=C(C#N)C#N]

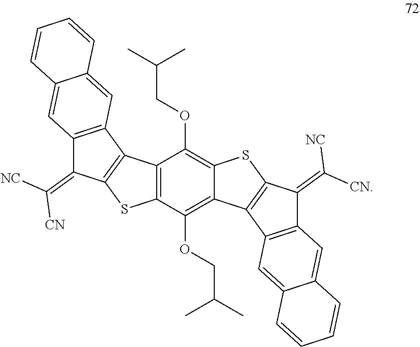 [SMILES: CC(C)COc1c2sc3c(c2c(OCC(C)C)c2sc4c(c12)-c1cc2ccccc2cc1C4=C(C#N)C#N)-c1cc2ccccc2cc1C3=C(C#N)C#N]

14. An organic photodetector comprising:
a first electrode;
a second electrode facing the first electrode;
an activation layer between the first electrode and the second electrode; and
the condensed cyclic compound of claim 1.

15. The organic photodetector of claim 14, wherein the condensed cyclic compound is in the activation layer.

16. The organic photodetector of claim 14, wherein the activation layer is to absorb green light or red light.

17. The organic photodetector of claim 14, wherein the first electrode is an anode,
the second electrode is a cathode,
the organic photodetector further comprises a hole transport region between the activation layer and the first electrode, and
the hole transport region comprises a hole injection layer, a hole transport layer, an auxiliary layer, an electron blocking layer, or any combination thereof.

18. The organic photodetector of claim 14, wherein the first electrode is an anode,
the second electrode is a cathode,
the organic photodetector further comprises an electron transport region between the activation layer and the second electrode, and
the electron transport region comprises a buffer layer, a hole blocking layer, an electron transport layer, an electron injection layer, or any combination thereof.

19. An electronic apparatus comprising the organic photodetector of claim 14.

20. The electronic apparatus of claim 19, further comprising a light-emitting device.
